(19) United States
(12) Patent Application Publication
(10) Pub. No.: US 2024/0186000 A1
(43) Pub. Date: Jun. 6, 2024

(54) SYSTEMS AND METHODS FOR DETECTING A DISEASE CONDITION

(71) Applicant: Icahn School of Medicine at Mount Sinai, New York, NY (US)

(72) Inventors: John Martignetti, Chappaqua, NY (US); Peter Dottino, New York, NY (US); Boris Reva, New York, NY (US)

(21) Appl. No.: 17/769,485

(22) PCT Filed: Oct. 16, 2020
(86) PCT No.: PCT/US20/56166

§ 371 Date: Apr. 15, 2022

Related U.S. Application Data
(60) Provisional application No. 62/916,103, filed on Oct. 16, 2019.

(51) Int. Cl. G16H 50/20; G01N 33/574; G01N 33/68; G16H 50/30

(52) U.S. Cl. CPC G16H 50/20; G01N 33/57442; G01N 33/57449; G01N 33/6854; G16H 50/30

(57) ABSTRACT

Systems and methods for evaluating an ovarian or uterine disease condition in a subject are provided. A uterine lavage fluid sample from the subject is obtained. For each autoantibody species in a first set of autoantibody species, a corresponding abundance value for the respective autoantibody species in the uterine lavage fluid sample is determined, thereby obtaining an autoantibody abundance dataset for the subject. The autoantibody abundance dataset is input into a classifier trained to distinguish between at least two states of the ovarian or uterine disease condition based on at least abundance values for the first set of autoantibody species. The classifier thereby obtains a probability or likelihood that the subject has a particular state of an ovarian or uterine disease condition.

### CROSS REFERENCE TO RELATED APPLICATION

This application claims priority to U.S. Provisional Patent Application No. 62/916,103, entitled “Systems and Methods for Detecting a Disease Condition,” filed Oct. 16, 2019, which is hereby incorporated by reference.

### TECHNICAL FIELD

This specification describes a system using proteomic analysis to evaluate subjects for having a disease condition. It is based upon the collection of a biological sample, proteomic characterization of the sample, and application of a machine learning approach to assign a risk score between two different states of disease. More specifically, the two states are absence or presence of, e.g., cancer, a precancerous lesion, or a non-cancerous condition.

### BACKGROUND

Cancer is a leading cause of death worldwide. Given that early stage solid cancers, those that are still localized to their site of origin, can generally be cured by surgery alone (see Siegel et al., 2018 C A Cancer J Clin 68, 7-30), a major focus of cancer research has been detection of premetastatic and early stage cancer lesions.

Ovarian and endometrial cancers are cancers for which early detection would be expected to significantly increase survival. Typically, these cancers are first diagnosed at a late stage and exhibit aggressive phenotypes with poor survival rates. See Ledermann et al. et al. 2013 Annals of Oncology 24(Supplement 6), vi24-vi32 and Colombo et al. et al. 2011 Annals of Oncology 22(Supplement 6), vi35-vi39. For example, of all cases of ovarian cancer diagnosed each year, approximately 75% are classified at diagnosis as high-grade serous cancers, which have a poor prognosis, with a 5-year survival rate of 10% to 30%. See e.g., Bodurka et al 2012 Cancer, 3087-3094.

At present, there are no screening tests for ovarian or endometrial pre-metastatic lesions or cancer. Typically, patients are tested only after they present with symptoms, when the cancer is advanced and prognosis is poor, and existing test methods suffer in both sensitivity and specificity. See Nair et al., 2016 PLoS Med 13(12):e1002206.

There will be more than 80,000 diagnoses of ovarian (OvCA) and endometrial (EndoCA) cancers this year in the U.S., and it is estimated that they will result in the death of 26,000 women. Cancer stage at diagnosis directly dictates treatment options and is the primary determinant of overall survival. For both of these gynecologic cancers, detection of early-stage, localized disease is associated with 5-year survival rates over 90%, while diagnosis with late-stage, metastatic disease results in dramatically reduced 5-year survival rates of ˜25%. Nearly 80% of OvCA cases are detected in late stages when the cancer has already spread. Twenty-five % of women diagnosed with EndoCA have late-stage disease. OvCA, in particular, often progresses without overt symptoms and presents later in the course of disease with non-specific symptoms (for example, constipation or diarrhea). Diagnosis requires radiographic imaging (transvaginal and/or abdominal ultrasonography, CT, MRI and/or PET) followed by radical cytoreductive surgery. In addition, these cancers disproportionally affect ethnically distinct populations. For example, 5-year survival rates for white and black women with EndoCA are 84% and 62%, respectively. Black women are also less likely to be correctly diagnosed with early-stage disease, and their survival rate at every stage is lower. Similar poorer outcomes are present in black women with OvCA. For all women, there are no screening tests for either of these two cancers or their known precursors, making detection at their earliest and curable stages nearly impossible.

Beyond cancer diagnoses, gynecologic diseases also account for a significant degree of morbidity, mortality and infertility. One-third of all women of reproductive age will experience nonmenstrual pelvic pain at some point in their lives (see Stratton 2020 UpToDate 5473 and Am College Obst. Gyn. 2020 Obstet Gynecol 135, e98-e109) and one-third of outpatient visits to gynecologists in the U.S. are for evaluation of abnormal uterine bleeding (see Kaunitz 2020 UpToDate 3263). These two non-specific symptoms, pelvic pain and abnormal bleeding, can be caused by a wide variety of non-pregnancy related conditions, including endometrial polyps, leiomyomas (uterine fibroids), adenomyosis, endometriosis, gynecological cancer, or pelvic inflammatory disease, among others. For many women, these symptoms accompany infertility which is reported in ˜10% of all US women and even higher percentages worldwide. See e.g. Wilkes et al. 2009 Family Practice 26, 269-274; Am College Obst. Gyn. 2019 Obstet Gynecol 133, e377-e384; and Stahlman 2019 Msmr 26, 20-27. For almost all of these women, these conditions result in a diagnostic odyssey wherein women struggle through multiple physicians over many years for a definitive diagnosis. See Nnoaham et al. 2011 Fertil Steril 96, 366-373; Ballard et a. 2006 Fertil Steril 86, 1296-1301; and Zondervan et al. 2020 N Engl J Med 382, 1244-1256.

In general, the diagnostic algorithm for pelvic pain, abnormal bleeding, and infertility begins with a detailed history and physical exam, followed by laboratory tests and imaging. Frequently the results from these tests are inconclusive, and women will need to undergo laparoscopy or hysteroscopy with dilation and curettage (D&C) for definitive diagnosis. Indeed, more than 198,000 operating room (OR)-based hysteroscopies are performed each year in the U.S. (see Hall et al 2017 Natl Health Stat Report 1-15 and Tam et al. 2016 J Min Invasive Gyn 23, S194), costing an average $14,600 per procedure or $2.9 B/year. OR-based hysteroscopy is performed under anesthesia by a surgeon and is associated with pain, risks of general anesthesia, and, indirectly, loss of time at work for the patient. In addition, a number of these common gynecologic conditions also disproportionally affect ethnically distinct populations. For example, leiomyomas are three times more prevalent in Black women, and these leiomyomas may be larger and more numerous causing worse symptoms and greater surgical treatment complications. See Baird, D. D., Dunson, D. B., Hill, M. C., Cousins, D. & Schectman, J. M. (2003). High cumulative incidence of uterine leiomyoma in black and white women: ultrasound evidence. Am J Obstet Gynecol 188, 100-107. PMID: 12548202; Marshall, L. M., Spiegelman, D., Barbieri, R. L. et al. (1997). Variation in the incidence of uterine leiomyoma among premenopausal women by age and race. Obstetrics & Gynecology 90, 967-973; Faerstein, E., Szklo, M. & Rosenshein, N. (2001). Risk factors for uterine leiomyoma: a practice-based case-control study. I. African-American heritage, reproductive history, body size, and smoking. Am J Epidemiol 153, 1-10. PMID: 11159139.

### SUMMARY

Accordingly, there is a need for screening and diagnostic tests for solid tumors that provide greater sensitivity and specificity that can detect precancerous changes, and that would allow diagnosis of solid tumors when still at a stage suitable for cure by surgical resection. There is a particular need for screening and diagnostic tests for endometrial and ovarian cancer. There is a particular need for screening and diagnostic tests for gynecologic diseases beyond cancer. The present disclosure addresses these and other needs by providing robust techniques for detecting whether a subject has a disease condition, e.g., cancer or non-cancerous disease.

As described herein, a novel machine learning method (ML) for classification of molecular profiles with autoantibody (AAb) profiling of blood samples and uterine lavage samples collected as part of the Gynecologic Cancer Translational Research Program (GCTRP; Icahn School of Medicine at Mount Sinai; New York, NY and Nuvance Health, Danbury, CT) to identify diagnostic biomarkers was developed. It has been demonstrated that AAb signatures can be used to differentiate between women with and without EndoCA or OvCA with accuracies of ˜90% or higher (area under receiver operating curve, AUROC=0.92). Another set of biomarkers was able to differentiate women with OvCA from those with EndoCA (AUROC=0.97). Different sets of biomarkers allowed us to differentiate women with and without complex atypical hyperplasia (a pre-cancerous condition) and women with and without specific gynecologic diseases including polyps, adenomyosis, leiomyoma, and endometriosis, which together are major causes of pelvic pain and infertility. Development of a highly sensitive and specific biomarker-based screening assay that could be offered to every woman at perimenopause or older and those with increased risk would enable early detection of OvCA and EndoCA and dramatically reduce the death rates from these devastating diseases. For early-stage OvCA, combined surgery and chemotherapy results in 5-year survival rates of >90%. For the earliest stages of EndoCA, medical management, without surgery, through progesterone or simple dilation and curettage, may provide cure.

In some embodiments, a single diagnostic test is provided for simultaneous screening for OvCA and EndoCA in asymptomatic women. In some embodiments, the test will consist of a panel of AAbs that together can distinguish between: (1) women with and without cancer, (2) OvCA (requiring surgery) from EndoCA (potential for no or minimal surgical management), and (3) less and more aggressive EndoCA (none vs more extensive surgical treatment and chemotherapy). Discovery that a collection of AAbs can be used to detect OvCA and EndoCA with high accuracy was made possible in part by >12 years of biobanking efforts. The GCTRP Biobank, represents a longitudinally collected, deeply clinically annotated set of fresh frozen primary and recurrent tumors, adjacent normal tissue, and blood samples, from >1,950 patients with >31,200 samples, all linked to patient outcome and treatments. Samples were collected by gynecologic oncologists with highly similar treatment practices and definitions; minimizing potential confounding, non-biological sources of treatment and survival differences. Quality and information content thresholds for biobanking and molecular analytics-based projects are in part demonstrated by participation in large scale projects like the NCI-funded Tumor Cancer Genome Atlas (TCGA) and Clinical Proteomic Tumor Analysis Consortium (CPTAC) studies.

Prior work by others on the interrelationships between AAbs and conditions like cancer has been limited by the size of the proteome screening library (for example, limited to a pre-selected fraction of the proteome), the specific isotypes included in the analysis, quantitation of AAb amounts, absence of post-translational modifications and validated protein folding, and lack of powerful analytic approaches. As described herein, comprehensive AAb profiles of each patient were obtained using CDI's HuProt proteome microarrays, a technology that allows simultaneous detection of IgG, IgA, and other AAbs to any of >21,000 full length, properly folded, human proteins with eukaryotic post-translational modifications, representing >81% of the human proteome. All recombinant proteins are expressed from sequence-confirmed plasmids then piezoelectrically printed with duplicate spots. Correct folding is confirmed using kinase autophosphorylation assays prior to screening. HuProt enables serum profiling of antibodies against three-dimensional antigens with a quantitative readout.

In some embodiments, the diagnostic assay described herein is based on a new proprietary application of a ML-based method for classification of molecular profiles. The underlying mathematic model allows the combination of imperfect signals of individual biomarkers into a significantly more powerful classification function that can differentiate molecular profiles of biologically different tumors or biospecimens. While the parent approach used gene expression levels as biomarkers, the current application will implement a new proprietary approach. In some embodiments, it replaces gene biomarkers with “pairwise biomarkers” defined as the differences between logarithms of abundance levels of pairs of autoantibodies (AAbs). This approach helps avoid batch effects because it uses relative expression values, rather than absolute values and significantly reduces the number of biomarkers that will be required for the commercial diagnostic panel. Classification accuracies have been compared with accuracies produced by 10 other well-established machine learning algorithms including Support Vector Machine and Random Forest. The current ML approach produced the most accurate classifications.

In accordance with some embodiments, a method for evaluating a gynecologic disease condition in a subject includes obtaining a uterine lavage fluid sample from the subject. The method further includes determining, for each autoantibody species in a first set of autoantibody species, a corresponding abundance value for the respective autoantibody species in the uterine lavage fluid sample. The method thereby obtains an autoantibody abundance dataset for the subject. The method also includes inputting the autoantibody abundance dataset into a classifier. The classifier is trained to distinguish between at least two states of the ovarian or uterine disease condition based on at least abundance values for the first set of autoantibody species. The classifier thereby obtains a probability or likelihood that the subject has a particular state of an ovarian or uterine disease condition.

In accordance with some embodiments, a method for evaluating an ovarian or uterine disease condition in a subject includes obtaining a uterine lavage fluid sample from the subject. The method includes determining, for each autoantibody species in a plurality of autoantibody species, a corresponding abundance value for the respective autoantibody species in the uterine lavage fluid sample. The method thereby obtains a master autoantibody abundance dataset for the subject. The method includes inputting a first subset of the master autoantibody abundance dataset into a first classifier. The first classifier is trained to distinguish between the presence of adenomyosis and the absence of adenomyosis based on at least abundance values for a first subset of the plurality of autoantibody species. The first classifier thereby obtains a probability or likelihood that the subject has adenomyosis. The method includes inputting a second subset of the master autoantibody abundance dataset into a second classifier. The second classifier is trained to distinguish between the presence of endometrial polyps and the absence of endometrial polyps based on at least abundance values for a second subset of the plurality of autoantibody species. The second classifier thereby obtains a probability or likelihood that the subject has endometrial polyps. The method includes inputting a third subset of the master autoantibody abundance dataset into a third classifier. The third classifier is trained to distinguish between the presence of leiomyoma and the absence of leiomyoma based on at least abundance values for a third subset of the plurality of autoantibody species. The third classifier thereby obtains a probability or likelihood that the subject has leiomyoma. The method also includes inputting a fourth subset of the master autoantibody abundance dataset into a fourth classifier. The fourth classifier is trained to distinguish between the presence of endometriosis and the absence of endometriosis based on at least abundance values for a fourth subset of the plurality of autoantibody species. The fourth classifier thereby obtains a probability or likelihood that the subject has endometriosis.

In accordance with some embodiments, a method for evaluating a disease condition in a subject includes obtaining a first biological fluid sample from the subject. The method includes determining, for each autoantibody species in a first set of autoantibody species, a corresponding abundance value for the respective autoantibody species in the first biological fluid sample. The method thereby obtains an autoantibody abundance dataset for the subject. The method further includes inputting the autoantibody abundance dataset into a classifier. The classifier is trained to distinguish between at least two states of the disease condition based on at least abundance values for the first set of autoantibody species. The classifier thereby obtains a probability or likelihood that the subject has a particular state of the disease condition.

In accordance with some embodiments, the method comprises (a) obtaining a biological sample from the subject, and (b) analyzing the biological sample for an abundance, E, of each autoantibody in a plurality of autoantibodies, thereby obtaining an autoantibody abundance dataset for the subject that includes an abundance of each autoantibody in the plurality of autoantibodies. The method continues with (c) filtering the autoantibody abundance dataset in accordance with a set of reference features, thereby obtaining a set of targeted autoantibody abundance levels for the subject. The method further includes (d) determining at least in part based on the set of targeted autoantibody abundance levels, a disease profile for the subject. The method proceeds by (e) applying the disease profile to a trained classifier, thereby obtaining a probability or likelihood from the trained classifier that the subject has the disease condition.

In some embodiments, the disease profile Vₛ for the tumor s is calculated as: Vₛ=Σₘ Aₘ·Eₘₛ. In some embodiments, m is a first autoantibody, Aₘ is a weight for autoantibody m, and Eₘₛ is an expression level of each autoantibody m in tumor s.

In some embodiments, the weight for each autoantibody, Aₘ, is calculated as: Aₘ˜Dₘ⁻¹Σₖ└Cₘₖ┘⁻¹Zₖ. In some embodiments, Dₘ is the standard deviation of expression of the autoantibody m, k is a second autoantibody, Cₘₖ is a pairwise correlation between expression of autoantibodies m and k, and Zₖ is a z-score for autoantibody k.

In some embodiments, filtering the autoantibody abundance dataset includes applying the overall ranked set of autoantibodies to a feature extraction method.

In some embodiments, the method includes (a) obtaining a lavage fluid sample from the subject (e.g., the biological sample comprises a lavage fluid sample). The method continues by (b) analyzing through a proteomics analysis, the lavage fluid sample for an abundance of each autoantibody in a plurality of autoantibodies using a protein for each autoantibody in the plurality of autoantibodies, thereby obtaining an autoantibody abundance dataset for the subject that includes an abundance of each autoantibody in the plurality of autoantibodies. The method continues by (c) filtering the autoantibody abundance dataset in accordance with a set of reference features, thereby obtaining a set of targeted autoantibody abundance levels for the subject. The method proceeds by (d) inputting the set of targeted autoantibody abundance levels into a trained classifier, thereby obtaining a probability or likelihood from the trained classifier that the subject has endometrial or ovarian cancer (e.g., the disease condition is early or pre-malignant endometrial or ovarian cancer).

In some embodiments, the biological sample includes lavage fluid (e.g., uterine lavage fluid, bladder lavage fluid, oral rinse, and lung washings), blood, urine, or cerebrospinal fluid.

In some embodiments, the proteomics analysis includes obtaining IgG and IgA profiles of the plurality of autoantibodies obtained from the lavage fluid sample. In some embodiments, the IgG and IgA profiles are combined, thereby determining the respective abundance level of each autoantibody in the plurality of autoantibodies.

In some embodiments, the set of reference features is selected from a list of predicted molecular pathways and/or cell type signatures in Table 1.

In some embodiments, the obtaining step (a) further includes extracting a plurality of nucleic acid sequence reads from the lavage fluid sample. In such embodiments, the analyzing step (b) further includes sequencing with a predetermined minimum coverage value the plurality of nucleic acid sequence reads targeted by a panel of genes, thereby obtaining a set of gene expression levels for the subject. In such embodiments, the inputting step (d) further includes inputting, for example, the set of gene expression levels, mutation profiles of genes, and clinicopathologic information (e.g., age, body mass index, race/ethnicity, and family history).

In some embodiments, the panel of genes includes at least 2 genes, at least 5 genes, at least 10 genes, at least 15 genes, or at least 20 genes.

In some embodiments, a stage of endometrial cancer includes stage 0 endometrial cancer, stage Ia endometrial cancer, stage Ib endometrial cancer, stage II endometrial cancer, stage III endometrial cancer, stage IV endometrial cancer, or pre-neoplastic condition.

In some embodiments, the trained classifier is a machine learning algorithm. Exemplary machine learning algorithms include a molecular signature algorithm, a neural network algorithm, a support vector machine algorithm, a decision tree algorithm, an unsupervised clustering model algorithm, a supervised clustering model algorithm, or a regression model or combination of machine learning algorithms

Another aspect includes a non-transitory computer readable storage medium and one or more computer programs embedded therein, the one or more computer programs comprising instructions which, when executed by a computer system, cause the computer system to perform a method evaluating a subject for a disease condition. An additional aspect includes a device for evaluating a subject for a disease condition comprising one or more processors, and memory storing one or more programs for execution by the one or more processors.

Another aspect includes a non-transitory computer readable storage medium and one or more computer programs embedded therein, the one or more computer programs comprising instructions which, when executed by a computer system, cause the computer system to perform a method evaluating a subject for a disease condition. An additional aspect includes a device for evaluating a subject for a disease condition comprising one or more processors, and memory storing one or more programs for execution by the one or more processors.

In another aspect, a classification method is provided. The classification method comprises obtaining (a), for each respective reference subject in a plurality of reference subjects, i) a first reference plurality of autoantibody abundance levels from a first biological sample, ii) a second reference plurality of autoantibody abundance levels from a second biological sample and iii) a corresponding indication of a respective cancer condition, wherein each autoantibody abundance level in the first biological sample is paired with an autoantibody abundance level from the second biological sample, thereby obtaining a set of resulting paired autoantibody abundance levels for each respective reference subject. The method continues by determining (b), for each respective reference subject, an overall ranked set of autoantibodies based on the set of resulting paired autoantibody abundance levels from each respective reference subject. The method includes applying (c) the overall ranked set of autoantibodies to a feature extraction method, thereby obtaining a subset of the overall ranked set of autoantibodies. The method proceeds by training an untrained classifier with at least i) the resulting paired autoantibody abundance levels for each respective reference subject for the subset of the overall ranked set of autoantibodies and ii) the corresponding indication of a respective cancer condition, thereby obtaining a trained classifier that evaluates a probability or likelihood that a test subject has a stage of endometrial or ovarian cancer.

In some embodiments, the respective cancer condition of each reference subject in a first set of the reference subjects in the plurality of reference subjects comprises non-cancer.

In some embodiments, the respective cancer condition of each reference subject in a second set of the plurality of reference subjects comprises stage 0 endometrial cancer, stage IA endometrial cancer, stage IB endometrial cancer, stage II endometrial cancer, stage III endometrial cancer, or stage IV endometrial cancer.

In some embodiments, the subset of the overall ranked set of autoantibodies corresponds to a list of predicted molecular pathways and/or cell type signatures in Table 1.

In some embodiments, obtaining (a) the subset of the overall ranked set of autoantibodies includes removing from the ranked set of autoantibodies one or more autoantibodies that do not meet a first criterion.

In some embodiments, the first criterion includes a p-value threshold, where ranked autoantibodies with p-values higher than the p-value threshold are removed.

Another aspect includes a non-transitory computer readable storage medium and one or more computer programs embedded therein, the one or more computer programs comprising instructions which, when executed by a computer system, cause the computer system to perform a classification method. An additional aspect includes a classification device comprising one or more processors, and memory storing one or more programs for execution by the one or more processors.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is a block diagram illustrating an example of a computing system in accordance with some embodiments of the present disclosure.

FIG. 2 illustrates a flowchart of a method for evaluating a subject for a disease condition, in accordance with some embodiments of the present disclosure.

FIG. 3 illustrates a flowchart of a method for evaluating a subject for a disease condition, in accordance with some embodiments of the present disclosure.

FIG. 4 illustrates ROC curves for training (402) and test samples (404) using pathway scores derived from IgG and IgA profiles in accordance with some embodiments of the present disclosure.

FIGS. 5A and 5B collectively illustrate the separation of cancer (black circles) and non-cancer (grey circles) samples based on pathway scores derived from IgG and IgA profiles in accordance with some embodiments of the present disclosure.

FIG. 6 illustrates ROC curves for training (602) and test (604) samples using pathway scores derived from IgG profiles in accordance with some embodiments of the present disclosure.

FIGS. 7A and 7B collectively illustrate the separation of cancer (black circles) and non-cancer (grey circles) samples based on pathway scores derived from IgG profiles in accordance with some embodiments of the present disclosure.

FIGS. 8A, 8B, and 8C are prior art from Rykunov et al 2016 Nuc Acids Res 44(11), e110 illustrating a) the selection of nominated driver genes associated with cancer type, b) ranking of autoantibodies in terms of significance and occurrence, and c) determining a molecular signature of a disease based on classification accuracy.

FIG. 9A illustrates ROC curves for training and test samples using sums of biomarker expression levels determined from plasma-derives autoantibody profiles, in accordance with some embodiments of the present disclosure. FIGS. 9B and 9C collectively illustrate the separation of cancer (black circles***) and non-cancer (grey circles***) samples based on biomarker scores determined from plasma-derived autoantibody profiles, in accordance with some embodiments of the present disclosure. The algorithm takes as input a dataset divided into two classes (e.g. cancer/benign, or OvCa/EndoCa) and a list of biomarkers, whose expression levels are differentially distributed between these two classes. A classification function that will optimize the separation between given diagnostic classes is then created as a weighted sum of biomarker expression levels, where weights are computed analytically (see e.g., Liu et al. 2018 Cell 173, 400-416 e411) using pairwise biomarker correlations. An original data set comprised of 135 AAb profiles (e.g., 45 profiles from women with cancer, 90 profiles from women without cancer) was repeatedly (e.g., 4096×) and randomly divided into approximately equal training and test sets. Biomarkers were differentially distributed between two classes in both sets were identified and ranked both by statistical power (e.g., by p-value) and by occurrence. The training set was used to determine biomarker weights and optimal classification thresholds to be tested in the independent test set. From the ranked list of candidate biomarkers, all possible sets of biomarkers (e.g., typically at least 35 biomarkers) were tested by adding biomarkers singly and in succession. Thus, for each “molecular signature” from a ranked list of candidate biomarkers and each sample, the probability of correct classification and average scoring were computed in multiple classification tests. These values were then used for computation of overall classification accuracies assessed by area under receiver operating curves (AUC) both for averaged classification scores and for probabilities (e.g., as shown in FIG. 9A). In multiple training/test samplings, no significant difference between the simplified and rigorous approaches was found (e.g., as shown in FIGS. 9B and 9C).

FIG. 10 illustrates a heatmap stratifying an optimized set of 24 biomarkers determined from plasma-derived autoantibody profiles, in accordance with some embodiments of the present disclosure. The heatmap demonstrates expression values of an optimal set of 24 biomarkers (e.g., ranked in descending order) in 135 samples that are sorted from left to right based on their testing score, with the left-most samples receiving classification scores of −15 (e.g., the highest confidence classification of “benign”) and the right-most samples receiving classification scores of 5 (e.g., the highest confidence classification of “cancer”). The green class information presents the known classification based on the patient's clinical history. Scores close to −15 and 5 are accurate (e.g., there are few to no misclassifications), while those scores closer to the center are less accurate (e.g., there are some misclassifications).

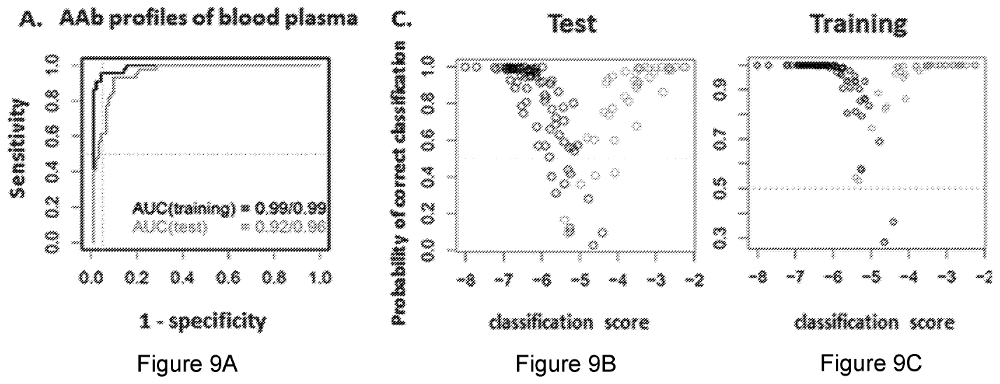

FIGS. 11A, 11B, and 11C illustrate classification of uterine lavage samples with regards to endometrial polyps (e.g., “polyps vs. no polyps”), in accordance with some embodiments of the present disclosure. FIG. 11A illustrates ROC curves for training (502) and test (504) samples using sums of biomarker expression levels determined from uterine-lavage autoantibody profiles, in accordance with some embodiments of the present disclosure. FIGS. 5B and 5C collectively illustrate the separation of cancer (black circles***) and non-cancer (grey circles***) samples based on biomarker scores determined from uterine-lavage autoantibody profiles, in accordance with some embodiments of the present disclosure. In FIGS. 11B and 11C, averaged probabilities of correct classification as functions of averaged scoring functions are presented, respectively. The characteristics were derived from ˜4000 individual classification tests, where the original data set of 80 samples was divided by random in training and test sets (e.g., where each of the training and test sets represent ˜50% of samples). The training set was used to determine biomarkers (e.g., differentially expressed AAbs) which were used to compute a classification scoring function (weighted sum of biomarkers' expression values) that was constructed to optimize separation of the training set into given clinical classes. Samples in the test set were then classified using the classification function of the training set (i.e. biomarkers, biomarker weights and classification threshold). Thus, in each classification test, each sample was classified in one of the given classes (training or test sets) and each sample was assessed by classification score. AUCs were derived for both averaged probabilities of correct classification and classification scores, respectively.

FIGS. 12A, 12B, and 12C illustrate classification of uterine lavage samples with regards to adenomyosis (e.g., “adenomyosis vs. no adenomyosis”), in accordance with some embodiments of the present disclosure. FIG. 12A illustrates ROC curves for training and test samples using sums of biomarker expression levels determined from uterine-lavage autoantibody profiles, in accordance with some embodiments of the present disclosure.

FIGS. 12B and 12C collectively illustrate the separation of cancer (black circles***) and non-cancer (grey circles***) samples based on biomarker scores determined from uterine-lavage autoantibody profiles, in accordance with some embodiments of the present disclosure.

FIGS. 13A, 13B, and 13C illustrate classification of uterine lavage samples with regards to leiomyoma (e.g., “leiomyoma vs. no leiomyoma”), in accordance with some embodiments of the present disclosure. FIG. 13A illustrates ROC curves for training and test samples using sums of biomarker expression levels determined from uterine-lavage autoantibody profiles, in accordance with some embodiments of the present disclosure.

FIGS. 13B and 13C collectively illustrate the separation of cancer (black circles***) and non-cancer (grey circles***) samples based on biomarker scores determined from uterine-lavage autoantibody profiles, in accordance with some embodiments of the present disclosure.

FIG. 14 illustrates a flowchart of a method for evaluating an ovarian or uterine disease condition in a subject, in accordance with some embodiments of the present disclosure.

FIG. 15 illustrates a flowchart of a method for evaluating an ovarian or uterine disease condition in a subject, in accordance with some embodiments of the present disclosure.

FIG. 16 illustrates a flowchart of a method for evaluating a disease condition in a subject, in accordance with some embodiments of the present disclosure.

FIG. 17 provides a summary of classification tests conducted for various combinations of diagnoses. EC, OvCA stand for endometrial and ovarian cancers, respectively. Each row (1-7) contains information on a single classification function, including number of samples classified as either Class 1 or Class 2 and associated AUC for both the test and training sets.

FIG. 18 provides a summary of classification tests conducted for various combinations of diagnoses. Each row contains information on a single classification function, including number of samples classified as either Class 1 or Class 2 and associated AUC for both the test and training sets.

FIGS. 19A and 19B collectively illustrate separation of adenomyosis vs non-adenomysosis: IgA. FIG. 19A shows computation of overall classification accuracies assessed by area under receiver operating curves (AUC) both for averaged classification scores and for probabilities. FIG. 19B shows a heatmap demonstrating expression values of an optimal set of 33 biomarkers (top to bottom) in ˜320 samples that are sorted from left to right based on their testing score, with the left-most samples receiving highest confidence of non-adenomyosis benign to the right most samples receiving highest confidence classification of adenomyosis. The magenta colored Class information presents the known classification based on the patient's clinical history.

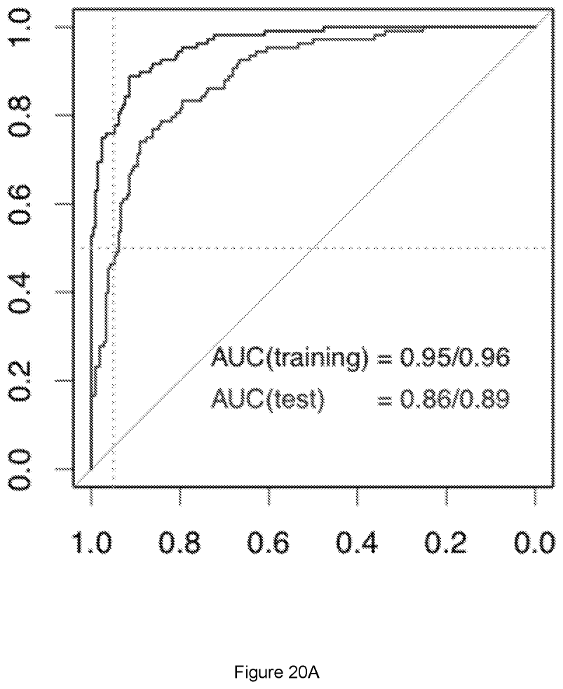

FIGS. 20A and 20B collectively illustrate separation of polyps vs non-polyps: IgA. FIG. 20A shows computation of overall classification accuracies assessed by area under receiver operating curves (AUC) both for averaged classification scores and for probabilities. FIG. 20B show a heatmap demonstrating expression values of an optimal set of 29 biomarkers (top to bottom) in ˜320 samples that are sorted from left to right based on their testing score, with the left-most samples receiving highest confidence of non-polyps to the right most samples receiving highest confidence classification of polyps. The magenta colored Class information presents the known classification based on the patient's clinical history.

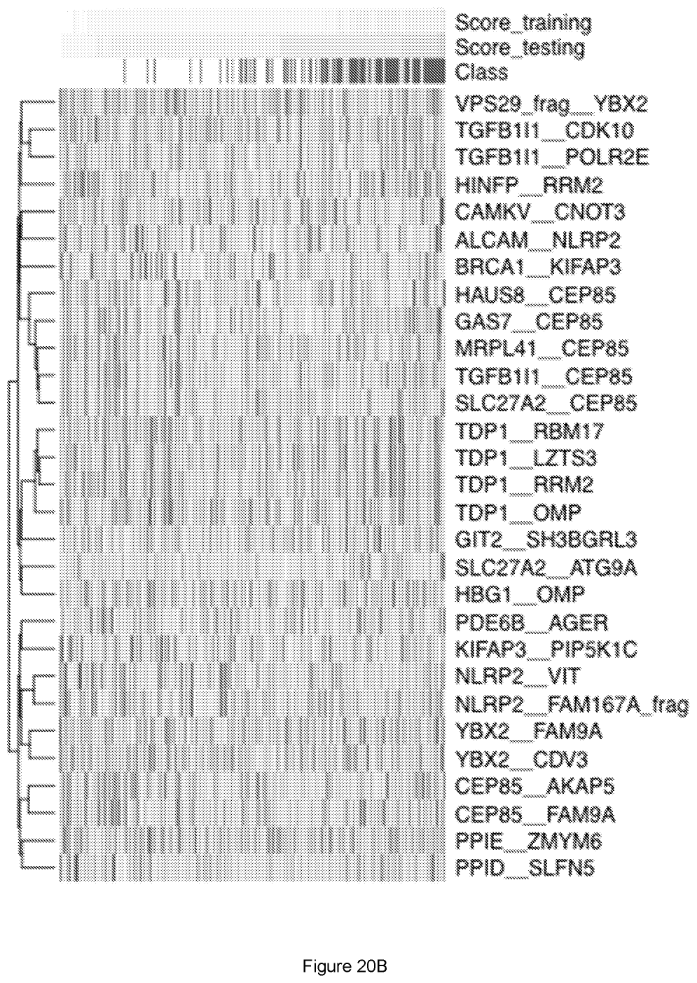

### DETAILED DESCRIPTION

There is a clear unmet need for a screening test to detect ovarian (OvCA) and endometrial (EndoCA) cancers prior to symptom onset and ultimate disease spread. More than 80,000 women in the U.S. will be diagnosed with one of these cancers this year and >26,000 women will die from their disease. OvCA is overwhelmingly detected in its late metastatic stage and this failure to detect early-stage OvCA is directly linked to poor outcome. EndoCA, the most common cancer of the female genital tract worldwide is one of the few cancers in which incidence and death rates continue to rise. Moreover, EndoCA has the greatest racial disparities among all cancers in detection and survival, with significantly worse outcomes for women of color. The ability to simultaneously screen for and detect these two cancers early through a simple, single blood test would dramatically change clinical management and treatment, saving tens of thousands of lives each year.

Based on the current lack of biomarkers, no screening programs exist or are currently recommended for these two cancers. Two large, randomized controlled trials (PLCO, n=78,00071,72 and UKCTOCS, n=202,63873) have investigated the potential of using a combination of cancer antigen 125 (CA 125) and transvaginal ultrasound (TVU) for OvCA screening; however, OvCA mortality was not significantly different between intervention and control groups. Based on the failures of these two trials, and a lack of alternate, effective novel biomarkers/diagnostics, the US Preventative Services Task Force recommends against OvCA screening.

Given the limitations of the currently available approaches, efforts continue to search for new screening biomarkers. The most effective tests under development incorporate multiple biomarkers. A subset of samples from the UKCTOCS study (n=80 women) were analyzed and 5 additional longitudinal biomarkers were identified that together improve upon CA. A test called PapSEEK that analyzes DNA in fluids obtained during a Pap test detects mutations in 18 genes and assesses aneuploidy; however, PapSEEK only displayed a sensitivity of 33% for early-stage ovarian cancer (specificity of ˜99%) when used alone (n=245 women with OvCA; 382 with EndoCA). The sensitivity increased to 63% (95% CI, 51 to 73%) when combined with plasma biochemical testing. While a number of approaches demonstrate relatively good detection of late-stage cancers these tests remain unsatisfactory for early-stage/pre-metastatic detection. As noted above, detection of early-stage cancers offers the opportunity for improved treatments and outcomes. There are a number of registered clinical trials currently recruiting or active; however, many are in the discovery phase and involve approaches not ideal for development of screening tests for early-stage identification such as mass spectrometry, or collection of samples under anesthesia. Tests that rely exclusively on identification of cancer mutations are also unlikely to be effective for screening. Published and unpublished studies from our group and others using next-generation sequencing of cellular and cell-free DNA collected from uterine lavage, tissue samples, and blood revealed a previously unknown and prevalent landscape of cancer driver mutations in women without cancer, illuminating the need for additional information beyond DNA mutation analysis.

To overcome these challenges, the disclosure is focused on developing a multiple-biomarker screening assay that concurrently uses OvCA- and EndoCA-specific AAbs as biomarkers. Finite sets of AAbs have been investigated as potential biomarkers for a number of disorders in part due to the immune system's critical role in responding to disease; in total, hundreds of tumor-associated AAbs (TAAs) have been identified across multiple cancers.

Efforts by other groups to identify diagnostic AAbs for OvCA can best be viewed as preliminary efforts to demonstrate proof-of-concept given the low AAb numbers interrogated or methods of analysis. A 2017 systematic review describes 29 studies that identified or evaluated a total of 85 different AAbs (contrasted with our 21,000 and multiple Ig subtypes), mostly from preselected subsets. Eighteen studies analyzed the potential of one AAb to identify OvCA, while 11 studies reported results for multiple AAbs (2-15 AAbs per panel). Only 10 of these studies used an unbiased screening approach to identify potentially diagnostic AAbs, with the remainder focusing on a preselected group of candidate AAbs. None used our unbiased whole proteome approach and none the ML analytic tool described herein. The most robust studies used methods to screen several thousand human proteins to identify antigens for diagnostic AAbs directly from human sera or plasma but their sample numbers were low. The largest study analyzed 94 cases of serous OvCA (95% stage III/IV) and 90 controls (patients with/without concurrent benign disease) to identify AAbs, and 50 cases (30 non-serous; 20 low CA125 OvCA) and 45 controls for validation. They identified 12 potential autoantigens with sensitivities ranging from 13-22% at >93% specificity, and 3 AAbs with AUC levels >60%. Taken together these studies support the idea that a panel of AAb biomarkers could be used for diagnosis of OvCA; however, none of them describe the development or testing of a strong panel that could be advanced to the clinic.

To address these and other needs, the present disclosure leverages access to >12 years of longitudinally collected and deeply annotated plasma samples biobanked through the Gynecologic Cancer Translational Research Program (Icahn School of Medicine at Mount Sinai; New York, NY and Nuvance Health, Danbury, CT). Using CDI Labs HuProt™ Human Proteome Array (Baltimore, MD) autoantibody (AAb) profiling of 135 deeply annotated plasma samples was performed and applied an iteration of a novel machine learning (ML) method for classification of molecular profiles to identify diagnostic AAb signatures. As described herein, hundreds of AAb markers differentially expressed between clinically relevant patient subtypes were identified. Further it was determined that a subset of <20 biomarkers can be used for construction of classification signatures capable of differentiating between the following diagnoses: 1. cancer and no cancer with accuracies of ˜90% or higher (area under receiver operating curve, AUROC=0.92), 2. OvCA from EndoCA (AUROC=0.97), and 3. less aggressive type I and more aggressive type II EndoCA subtypes.

Conventionally, this would require that each patient sample is screened against the entire 21,000 protein human proteome, which while extremely powerful, is prohibitively expensive, inefficient, and complicates the process of assigning a diagnostic risk score. However, our preliminary data further indicates that we can refine this screening panel to a minimum and common set of ˜100 biomarkers to screen all women. Advantageously, in some embodiments, this disclosure provides a single, affordable, easy-to-use, high confidence cancer biomarker panel that can be used to screen all peri-menopausal women and older.

Gynecologic diseases are those diseases that involve the female reproductive track. These diseases and health conditions include both benign and malignant tumors including endometrial and ovarian cancers; premalignant conditions such as endometrial hyperplasia and cervical dysplasia, benign (i.e. non-cancerous conditions) including polyps, ovarian cysts, fibroids and adenomyosis; endometriosis (the implantation of ectopic endometrial tissue outside the uterus, resulting in symptoms including infertility, dysmenorrhea and pelvic pain), pregnancy-related diseases and infertility, menopause, pelvic inflammatory diseases and infection, and even endocrine diseases which relate to the female reproductive tract, for example primary and secondary amenorrhea, polycystic ovary syndrome and premature ovarian failure.

The distinct gynecologic diseases may themselves have broader downstream health ramifications which result in diagnostic odysseys taking up years of physicians visits and a range of diagnostic tests. For example, one-third of all women of reproductive age will experience nonmenstrual pelvic pain at some point in their lives [Stratton, P. (2020). Evaluation of acute pelvic pain in nonpregnant adult women. UpToDate 5473. PMID.; American College of Obstetricians and Gynecologists. (2020). Chronic Pelvic Pain: ACOG Practice Bulletin, Number 218. Obstet Gynecol 135, e98-e109. PMID: 32080051.] and one-third of outpatient visits to gynecologists in the United States are for evaluation of abnormal uterine bleeding [Kauntiz, A. M. (2020). Approach to abnormal uterine bleeding in nonpregnant reproductive-age women. UpToDate 3263.] These two non-specific symptoms, pelvic pain and abnormal bleeding, can be caused by a wide variety of non-pregnancy related conditions, including endometrial polyps, leiomyomas (uterine fibroids), adenomyosis, endometriosis, gynecological cancer, or pelvic inflammatory disease, among others. For many women, a number of these conditions also result in infertility which is reported in ˜10% of all US women and even higher percentages worldwide [Wilkes, S., Chinn, D. J., Murdoch, A. & Rubin, G. (2009). Epidemiology and management of infertility: a population-based study in UK primary care. Family practice 26, 269-274; Centers for Disease Control and Prevention. National Center for Health Statistics: Infertility, https://www.cdc.gov/nchs/fastats/infertility.htm; American College of Obstetricians and Gynecologists. (2019). Infertility Workup for the Women's Health Specialist: ACOG Committee Opinion, Number 781. Obstet Gynecol 133, e377-e384. PMID: 31135764.; Stahlman, S. & Fan, M. (2019). Female infertility, active component service women, U.S. Armed Forces, 2013-2018. Msmr 26, 20-27. PMID: 31237765.]

For almost all of these women, these conditions result in a diagnostic odyssey wherein women struggle through multiple physicians over many years for a definitive diagnosis. For example, on average, women with endometriosis consult seven physicians prior to diagnosis [Nnoaham, K. E., Hummelshoj, L., Webster, P. et al. (2011). Impact of endometriosis on quality of life and work productivity: a multicenter study across ten countries. Fertil Steril 96, 366-373.e368. EMS48415. PMC3679489; Ballard, K., Lowton, K. & Wright, J. (2006). What's the delay? A qualitative study of women's experiences of reaching a diagnosis of endometriosis. Fertil Steril 86, 1296-1301. PMID: 17070183; Zondervan, K. T., Becker, C. M. & Missmer, S. A. (2020). Endometriosis. N Engl J Med 382, 1244-1256. PMID: 32212520].

In general, the diagnostic algorithm for pelvic pain, abnormal bleeding and infertility begins with a detailed history and physical exam, followed by laboratory tests and imaging (sonohysterogram, transvaginal and transabdominal ultrasound, MRI). Frequently the results from these tests are inconclusive, and women will need to undergo laparoscopy or hysteroscopy with dilation and curettage (D&C) for definitive diagnosis. Indeed, >198,000 operating room (OR)-based hysteroscopies are performed each year in the U.S. [Hall, M. J., Schwartzman, A., Zhang, J. & Liu, X. (2017). Ambulatory Surgery Data From Hospitals and Ambulatory Surgery Centers: United States, 2010. Natl Health Stat Report, 1-15. PMID: 28256998; Tam, T., Archill, V. & Lizon, C. (2016). Cost Analysis of In-Office versus Hospital Hysteroscopy. Journal of minimally invasive gynecology 23, S194], costing an average $14,600 per procedure or $2.9 B/year. OR-based hysteroscopy is performed under anesthesia by a surgeon and is associated with pain, risks of general anesthesia, and indirectly, loss of time at work for the patient. Having a diagnostic test

A number of these common gynecologic conditions also disproportionally affect ethnically distinct populations. For example, leiomyomas are 3× more prevalent in Black women and these leiomyomas may be larger and more numerous causing worse symptoms and greater surgical complications [Baird, D. D., Dunson, D. B., Hill, M. C., Cousins, D. & Schectman, J. M. (2003). High cumulative incidence of uterine leiomyoma in black and white women: ultrasound evidence. Am J Obstet Gynecol 188, 100-107. PMID: 12548202; Marshall, L. M., Spiegelman, D., Barbieri, R. L. et al. (1997). Variation in the incidence of uterine leiomyoma among premenopausal women by age and race. Obstetrics & Gynecology 90, 967-973.; Faerstein, E., Szklo, M. & Rosenshein, N. (2001). Risk factors for uterine leiomyoma: a practice-based case-control study. I. African-American heritage, reproductive history, body size, and smoking. Am J Epidemiol 153, 1-10. PMID: 11159139].

In some embodiments, the methods described herein provides a diagnostic risk score, based on either blood and/or uterine lavage fluid analysis, that can identify an underlying gynecologic disease. This disease can be present in either an asymptomatic (i.e. a screening test) or asymptomatic (i.e. a diagnostic test) woman. These diagnostic risk scores will provide clinically actionable information in the form of guidance towards disease-specific treatment.

For example, for a female who is experiencing acute or chronic pelvic or abdominal pain, uterine bleeding, and/or infertility part of their current gold-standard diagnostic evaluation today by either their internist, general practitioner, reproductive specialist or gynecologist could require radiologic (CT, MRI, PET scan, transabdominal ultrasound) examination coupled with invasive operating room-based tissue biopsy (dilation and curettage; D&C) for diagnosis. In this context, and instead using our method at the start of a patient's diagnostic evaluation, a blood sample and/or uterine lavage fluid sample would be obtained for analysis. Depending on the disease identified, clinically actionable information in the form of guidance towards disease-specific treatment would then be delivered by the method's risk score. For example, if a risk score suggesting endometriosis was identified by the blood and/or uterine lavage-based test, the patient could avoid the need for additional diagnostic procedures including ultrasound evaluation, MRI and surgical laparoscopy. Instead, with our liquid biopsy based diagnosis, medical management for pain could be provided as well as medical management to directly treat the underlying disease, endometriosis. Medical management, avoiding surgery, could include the use of hormonal contraceptives, gonadotropin-releasing hormone (Gn-RH) agonists and antagonists, progestin therapy and aromatase inhibitors. Thus, in this example of a symptomatic patient of unknown disease etiology, the use of our method provides clinically actionable information capable of guiding day-to-day decision-making. It avoids the necessity for radiologic and surgical interventions to generate a diagnosis. Moreover, our method provides an opportunity to treat a gynecologic disease with medical management instead of surgical intervention which has historically included surgery to remove the uterus (hysterectomy) and both ovaries (oophorectomy).

Alternatively, if the diagnostic method identified a high risk score for ovarian cancer, that patient would be immediately sent from their internist, general practitioner, reproductive specialist or gynecologist to a specialist in diagnosing and treating gynecologic cancers. The directed transfer of care from a generalist practitioner to a cancer specialist would save time, avoid the intervening use of non-critical and expensive examinations, and as has been shown, treatment of women with gynecologic cancers by gynecologic oncologists and in specialized centers results in markedly improved outcomes for the patient [doi: 10.1016/j.ygyno.2007.02.030; doi: 10.1093/jnci/djj019; doi: 10.1097/01.AOG.0000265207.27755.28]

Finally, and given the costs of the diagnostic tests involved, inequalities of healthcare distribution, the limited geographic availability of and disproportionate distribution of the expertise/cost of trained operators/skilled physicians and equipment for diagnostic testing, our biomarker method requiring a blood sample or uterine lavage has the capacity to be performed in a general practitioners' office, performed by physicians' assistants or nurse practitioners, thus democratizing the overall diagnostic experience.

Development of a minimally invasive test that will efficiently diagnose the cause of these non-specific symptoms or triages women most likely to benefit from hysteroscopy or other invasive definitive testing would simultaneously minimize diagnostic delays, unnecessary surgeries, and possible loss of fertility, while improving outcomes and multiple burdens on the healthcare system. The methods described herein provide for a diagnostic test used to detect disease conditions in subjects. Particularly relevant disease conditions are early stage endometrial and ovarian cancers. Specifically, the methods enable testing a biological sample (e.g., lavage fluid) from a patient to distinguish between two or more different disease conditions, in particular between ovarian and endometrial cancer or between ovarian and/or ovarian cancer and non-cancer (e.g., evaluate a subject for a stage of a particular cancer condition or evaluate a subject for cancer vs non-cancer). In some embodiments, the methods described herein also provide for testing a biological sample to determine a probability or likelihood that a patient has a disease condition. In some embodiments, the method determines a probability or likelihood that a patient has a cancer of the uterus and/or female reproductive system (e.g., endometrial, cervical, or ovarian cancer). In some embodiments, the method determines a probability or likelihood that a patient has a non-cancerous disease of the uterus and/or female reproductive system (e.g., endometriosis, polyps, etc.).

This invention analyzes biological samples, such as lavage analytes, by combining screening for IgG and IgA autoantibodies, for example using a human proteome array, with a novel computational classifier. The methods described herein can be used for evaluation of disease conditions in both symptomatic and asymptomatic individuals (e.g., a patient does not need to exhibit one or more symptoms of ovarian or endometrial cancers). In particular, these methods can be performed as part of an annual or other screening (e.g., concurrent with a pap or STD test). Through early detection of many disease conditions, patients can receive appropriate treatment sooner. For some cancers in particular, for example ovarian and endometrial cancers, early detection contributes to significant increases in survival rates of patients.

This invention identifies an optimized panel of biomarkers (see e.g., autoantibodies in Example 2) to provide for an affordable, laboratory-based diagnostic test that will significantly reduce the number of women who will need to undergo laparoscopy or hysteroscopy with D&C for definitive diagnosis, enabling early treatment of disease and reducing the significant psychological and financial burden of diagnoses that otherwise can take years.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

Unless defined otherwise, all technical and scientific terms used herein have the meaning commonly understood by a person skilled in the art to which this invention belongs. The following references provide one of ordinary skill in the art with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991); Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the presently disclosed subject matter. As used herein, the following terms have the meanings ascribed to them below, unless specified otherwise.

As used herein, “gynecologic diseases” are those diseases that involve the female reproductive track. These diseases and health conditions include both benign and malignant tumors including endometrial and ovarian cancers; premalignant conditions such as endometrial hyperplasia and cervical dysplasia, benign (i.e. non-cancerous conditions) including polyps, ovarian cysts, fibroids and adenomyosis; endometriosis (the implantation of ectopic endometrial tissue outside the uterus, resulting in symptoms including infertility, dysmenorrhea and pelvic pain), pregnancy-related diseases and infertility, menopause, pelvic inflammatory diseases and infection, and even endocrine diseases which relate to the female reproductive tract, for example primary and secondary amenorrhea, polycystic ovary syndrome and premature ovarian failure.

As used herein, the terms “antibody” and “antibodies” refer to antigen-binding proteins of the immune system. In certain embodiments, an antibody can be produced by an individual's own immune system that binds to one or more of the individual's own proteins (e.g., self-antigens). Such antibodies are further defined as “autoantibodies.” See Garaud et al. et al. 2018 Front Immunol 9:2660. IgG and IgA are examples of high-affinity, somatically mutated autoantibodies (e.g., AAbs). Accordingly, as used herein, the abundance of an autoantibody species refers to the abundance of antibodies found in a biological sample from a subject, e.g., a uterine lavage fluid, that specifically bind to a molecular target, e.g., as determined using a proteomic analysis. It is expected that the abundance of some autoantibody species will include measurements of different autoantibodies, each of which specifically binds to the same molecular target.

As used herein, the term “lavage fluid” refers to a biological sample that is collected from a body cavity of a subject. In particular, “uterine lavage fluid” refers to a biological sample collected from a subject's uterus (e.g., via one or more washings). Lavage fluid can be used to test or screen for one or more disease conditions. See e.g., Nair et al., 2016 PLoS Med 13(12):e1002206 and Meyer et al. et al. 2011 Eur Respir J 38, 761-769. In certain circumstances, the use of lavage fluid is a less invasive method of screening for disease (e.g., as compared to other biopsy methods).

As used herein, the term “mutation” refers to permanent change in the DNA sequence that makes up a gene. In certain embodiments, mutations range in size from a single DNA building block (DNA base) to a large segment of a chromosome. In certain embodiments, mutations can include missense mutations, frameshift mutations, duplications, insertions, nonsense mutation, deletions, and repeat expansions. In certain embodiments, a missense mutation is a change in one DNA base pair that results in the substitution of one amino acid for another in the protein made by a gene. In certain embodiments, a nonsense mutation is also a change in one DNA base pair. Instead of substituting one amino acid for another, however, the altered DNA sequence prematurely signals the cell to stop building a protein. In certain embodiments, an insertion changes the number of DNA bases in a gene by adding a piece of DNA. In certain embodiments, a deletion changes the number of DNA bases by removing a piece of DNA. In certain embodiments, small deletions can remove one or a few base pairs within a gene, while larger deletions can remove an entire gene or several neighboring genes. In certain embodiments, a duplication consists of a piece of DNA that is abnormally copied one or more times. In certain embodiments, frameshift mutations occur when the addition or loss of DNA bases changes a gene's reading frame. A reading frame consists of groups of 3 bases that each code for one amino acid. In certain embodiments, a frameshift mutation shifts the grouping of these bases and changes the code for amino acids. In certain embodiments, insertions, deletions, and duplications can all be frameshift mutations. In certain embodiments, a repeat expansion is another type of mutation. In certain embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In certain embodiments, a repeat expansion is a mutation that increases the number of times that the short DNA sequence is repeated.

As used herein, the term “sample” refers to a biological sample obtained or derived from a source of interest, as described herein. In certain embodiments, a source of interest comprises an organism, such as an animal or human. In certain embodiments, a biological sample is a biological tissue or fluid. Non-limiting examples of biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, etc.

As used herein, the term “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested). In preferred embodiments, the subject is a human.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration, or palliation of the disease condition, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

It will also be understood that, although the terms first, second, etc., may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Exemplary System Embodiments

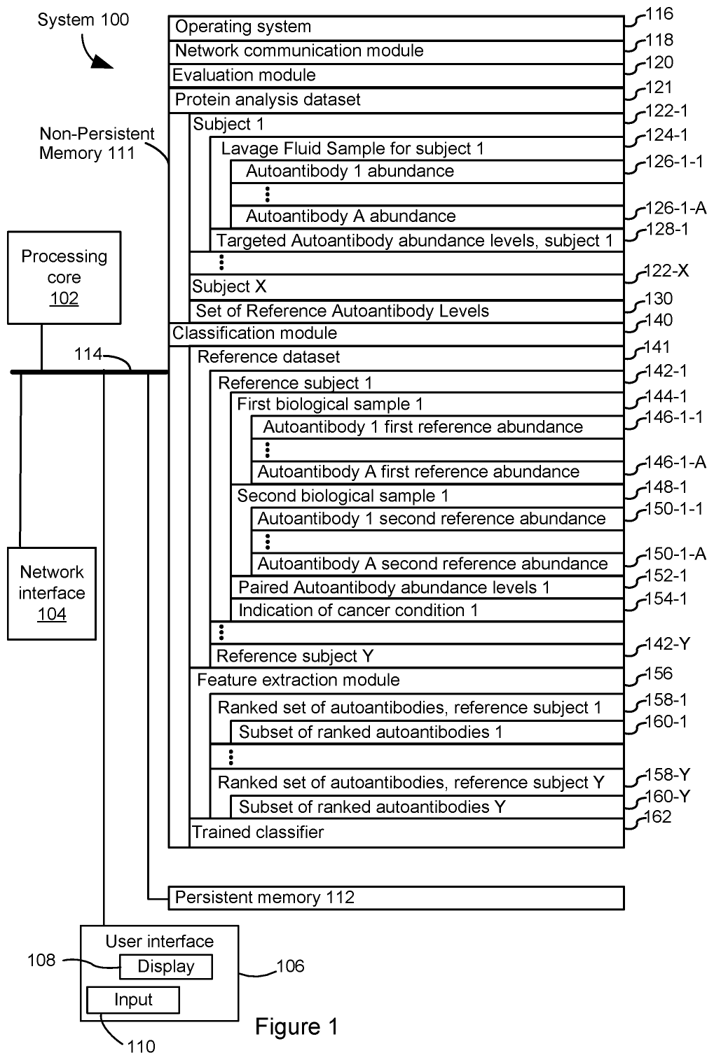

Details of an exemplary system are now described in conjunction with FIG. 1. FIG. 1 is a block diagram illustrating a system 100 in accordance with some implementations. The system 100 in some implementations includes at least one or more processing units CPU(s) 102 (also referred to as processors), one or more network interfaces 104, a display 106 having a user interface 108, an input device 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium, and stored thereon computer-executable executable instructions, which can be in the form of programs, modules, and data structures. In some implementations, the non-persistent memory 111 or alternatively the non-transitory computer readable storage medium stores the following programs, modules and data structures, or a subset thereof, sometimes in conjunction with the persistent memory 112:
an operating system 116, which includes procedures for handling various basic system services and for performing hardware-dependent tasks;
an optional network communication module (or instructions) 118 for connecting the system 100 with other devices and/or to a communication network;
an evaluation module 120 for evaluating a subject (e.g., subject 122-1, subject 122-2, . . . , and/or subject 122-X) for a stage of endometrial or ovarian cancer;
a protein analysis dataset 121 comprising, for each subject (e.g., subject 122-1), a plurality of antibody abundances (126-1-1, . . . 126-1-A) from a lavage fluid sample 124-1, and a set of targeted autoantibody abundance levels 128-1, and a set of reference autoantibody levels 130 (e.g., for filtering each plurality of autoantibody abundances to obtain the corresponding set of targeted autoantibody abundance levels for the respective subject); and
a classification module 140 for training a classifier to evaluate a subject for a stage of endometrial or ovarian cancer, comprising a reference dataset 141, a feature extraction module 156, and a trained classifier 162, where:
   the reference dataset 141 comprises, for each reference subject 142-1, 142-2, . . . 142-Y, a first biological sample (e.g., 144-1) and a second biological sample (e.g., 148-1), a set of paired autoantibody abundance levels 152-1, and an indication of a disease (e.g., cancer) condition for the respective reference subject 154-1, where the first biological sample includes a first reference abundance for each autoantibody in a plurality of autoantibodies (e.g., 146-1-1, . . . 146-1-A), and the section biological sample includes a second reference abundance for each autoantibody in the plurality of autoantibodies (e.g., 150-1-1, . . . 150-1-A); and
   the feature extraction module 156 comprises a ranked set of autoantibodies for each reference subject (e.g., 158-1, . . . 158-Y) and a subset of ranked autoantibodies (160-1, . . . , 160-Y).

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements are stored in a computer system other than the system 100, that is addressable by the system 100 so that the system 100 may retrieve all or a portion of such data when needed

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items can be separate. Moreover, although FIG. 1 depicts certain data and modules in non-persistent 111 or persistent memory 112, it should be appreciated that these data and modules, or portion(s) thereof, may be stored in more than one memory. For example, in some embodiments, at least the evaluation module 120, the protein analysis dataset 121, and the classification module 140 are stored in a remote storage device that can be a part of a cloud-based infrastructure. In some embodiments, at least the protein analysis dataset 121 is stored on a cloud-based infrastructure. In some embodiments, the evaluation module 120 and the classification module 140 can also be stored in the remote storage device(s).

While an example of a system in accordance with the present disclosure has been disclosed with reference to FIG. 1, methods in accordance with the present disclosure are now detailed.

Classifiers

In some embodiments, the methods described herein use autoantibody (also referred to herein as AAB or AAb) abundance values (also referred to herein as expression levels) to classify the state of a disorder, such as a gynecological disorder, in a subject. Generally, any classifier architecture can be trained for these purposes. Non-limiting examples of classifier types that can be used in conjunction with the methods described herein include a machine learning algorithm, molecular signature algorithm, a neural network algorithm, a support vector machine algorithm, a decision tree algorithm, an unsupervised clustering model algorithm, a supervised clustering model algorithm, or a regression model. In some embodiments, the trained classifier is binomial or multinomial.

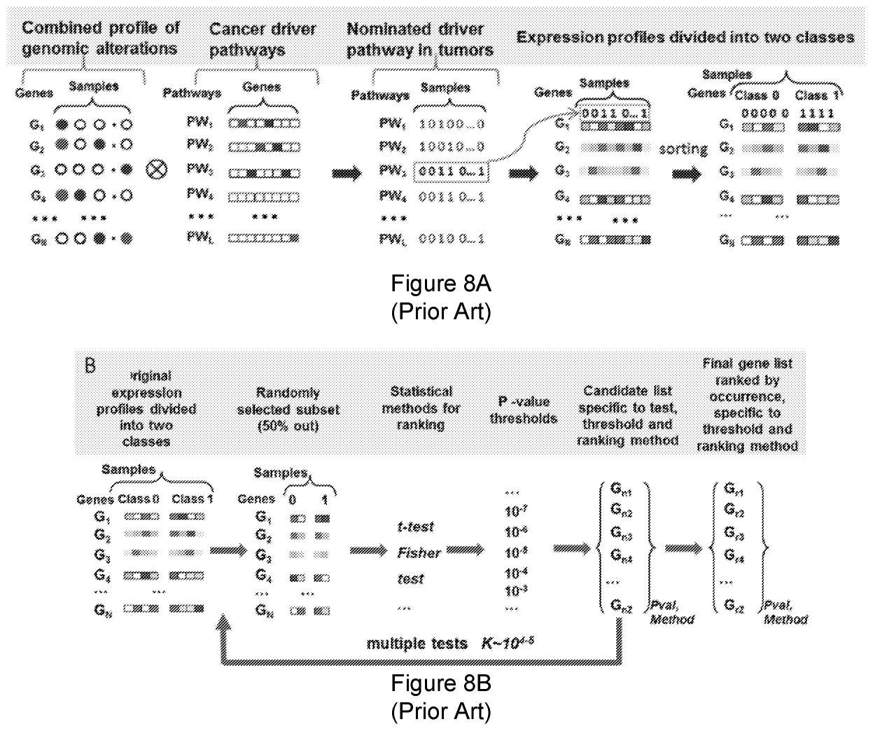

In some embodiments, the classifier includes a molecular signature model (MSM). See, Rykunov et al. et al. 2016 Nuc Acids Res 44(11), e110, the content of which is incorporated herein, by reference, in its entirety for all purposes. FIGS. 8A-8C illustrate an example of identifying molecular signatures with driver mutations (e.g., in accordance with MSM). As shown in FIG. 8A, in some embodiments, tumor molecular profiles from a plurality of subjects can be filtered using known driver alterations in molecular pathways, and different classes (e.g., for cancer vs. non-cancer or for two or more cancer conditions) of molecular expression profiles (e.g., molecular pathways with driver alterations) can be determined. FIG. 8B illustrates how potential molecular pathways and/or cell type signatures (e.g., the expression profile classes 1 and 0) can, in some embodiments, be ranked by occurrence (e.g., genes with expression levels that fall below predetermined p-value thresholds are discarded). In some embodiments, the overall set of molecular expression profiles can be subdivided (e.g., by randomly selecting 50% of the samples) into training and test datasets, and then the genes can be ranked using a t-test or a Fisher test (e.g., using the difference between the two expression profile classes 1 and 0). In some embodiments, this subdivision can be repeated one or more times (e.g., for 10⁴ or 10⁵ times) for determining a list of candidate molecular pathways and/or cell type signatures. These candidate molecular pathways and/or cell type signatures can be further evaluated for accuracy (e.g., the arithmetic mean of sensitivity and specificity) to determine a molecular signature comprising a set of gene expressions (e.g., average expression levels), for example as outlined in FIG. 8C.

Example logistic regression algorithms are disclosed in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, that can serve as the classifier for the instant methods are disclosed in See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

Support vector machine (SVM) algorithms that can serve as the classifier for the instant methods are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary-labeled data training set with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees (e.g., random forest, boosted trees) that can serve as the classifier for the instant methods are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can serve as the classifier for the instant methods is a classification and regression tree (CART). Other specific decision tree algorithms that can serve as the classifier for the instant methods include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

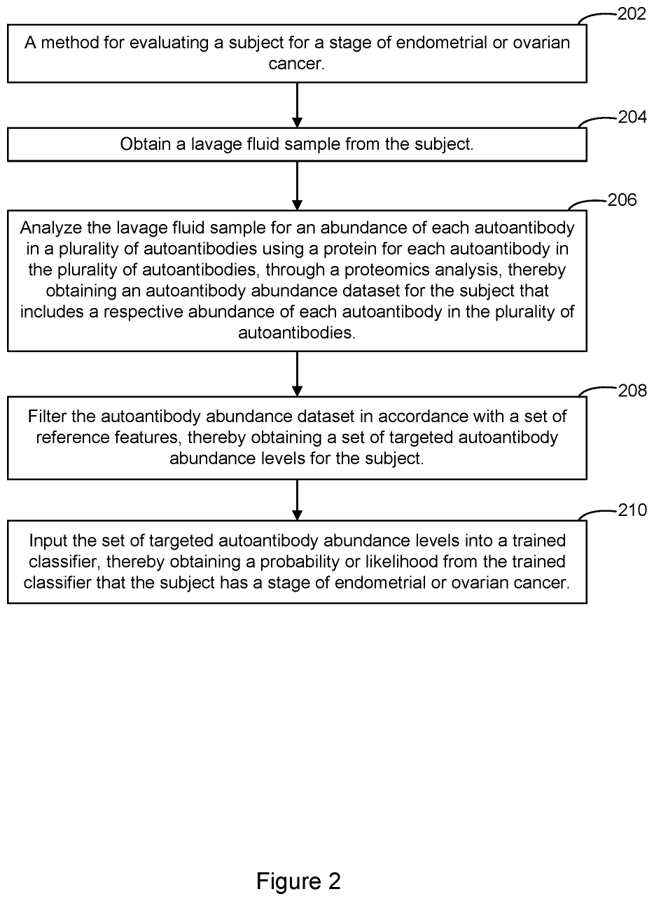

FIG. 2 illustrates an overview of the techniques in accordance with some embodiments of the present disclosure. In the described embodiments, various methods of collapsing nucleic acid base reads into base call are described. In some embodiments, the various methods are encoded in collapse classification module 120.

Classifier Features

In some embodiments of the methods described herein, e.g., methods 200, 1400, 1500, and 1600, classifiers use autoantibody abundance data to determine values for each of a set of autoantibody abundance features, which are used in the classification process. As described herein, in some embodiments, the autoantibody abundance features are abundance values for autoantibodies species, logs of the autoantibody abundance values, or a normalized abundance value thereof. For instance, in some embodiments, a normalization technique is applied to the autoantibody abundance values or logs thereof, such as scaling to a range, clipping, log scaling, or determining a z-score.

However, systemic errors and batch effects were encountered when the autoantibody abundance values, or logs thereof, were used to train a classifier. To define diagnostic biomarkers that are less sensitive to systematic errors and batch effects, a method was developed where the biomarkers and related classification functions can be applicable to a single sample. One way to satisfy this condition, i.e. minimization to a single sample, is to normalize all biomarkers by a computationally-derived “housekeeper” marker. Conventionally, a specific and pre-defined “housekeeping” gene, RNA sequence or protein, depending on the type of analyte being measured, is selected as the internal control. All subsequent measurements are then compared to that single housekeeper. However this method is non-trivial and can suffer from a number of issues including the necessity of a constant and non-zero expression value across all samples for that housekeeper and the ability to identify a priori such a housekeeper for the type of experiment being conducted. See, for example, Eisenberg E, Levanon E Y. Human housekeeping genes, revisited. Trends Genet. 2013 October; 29(10):569-74, Turabelidze A, Guo S, DiPietro L A. Importance of housekeeping gene selection for accurate reverse transcription-quantitative polymerase chain reaction in a wound healing model. Wound Repair Regen. 2010 September-October; 18(5):460-6, Tunbridge E M, Eastwood S L, Harrison P J. Changed relative to what? Housekeeping genes and normalization strategies in human brain gene expression studies. Biol Psychiatry. 2011 Jan. 15; 69(2):173-9, Wang Z, Lyu Z, Pan L, Zeng G, Randhawa P. Defining housekeeping genes suitable for RNA-seq analysis of the human allograft kidney biopsy tissue. BMC Med Genomics. 2019 Jun. 17; 12(1):86, Wisniewski J R, Mann M. A Proteomics Approach to the Protein Normalization Problem: Selection of Unvarying Proteins for MS-Based Proteomics and Western Blotting. J Proteome Res. 2016 Jul. 1; 15(7):2321-6, Kloubert V, Rink L. Selection of an inadequate housekeeping gene leads to misinterpretation of target gene expression in zinc deficiency and zinc supplementation models. J Trace Elem Med Biol. 2019 December; 56:192-197, and Chapman J R, Waldenström J. With Reference to Reference Genes: A Systematic Review of Endogenous Controls in Gene Expression Studies. PLoS One. 2015 Nov. 10; 10(11):e0141853, the contents of which are incorporated by reference herein, in their entireties, for all purposes.

In addition, given experimental differences in technical measurements, the “housekeeping” role may not be effectively translatable across different batches of test samples or testing under different conditions. See, for example, Asiabi P, Ambroise J, Giachini C, Coccia M E, Bearzatto B, Chiti M C, Dolmans M M, Amorim C A. Assessing and validating housekeeping genes in normal, cancerous, and polycystic human ovaries. J Assist Reprod Genet. 2020 October; 37(10):2545-2553, Maremanda K P, Sundar I K, Li D, Rahman I. Age-dependent assessment of genes involved in cellular senescence, telomere and mitochondrial pathways in human lung tissue of smokers, COPD and IPF: Associations with SARS-CoV-2 COVID-19 ACE2-TMPRSS2-Furin-DPP4 axis. medRxiv [Preprint], 2020 Jun. 16:2020.06.14.20129957, Bettencourt J W, McLaury A R, Limberg A K, Vargas-Hernandez J S, Bayram B, Owen A R, Berry D J, Sanchez-Sotelo J, Morrey M E, van Wijnen A J, Abdel M P. Total Protein Staining is Superior to Classical or Tissue-Specific Protein Staining for Standardization of Protein Biomarkers in Heterogeneous Tissue Samples. Gene Rep. 2020 June; 19:100641, Rai S N, Qian C, Pan J, McClain M, Eichenberger M R, McClain C J, Galandiuk S. Statistical Issues and Group Classification in Plasma MicroRNA Studies With Data Application. Evol Bioinform Online. 2020 Apr. 14; 16:1176934320913338, Dos Santos K C G, Desgagné-Penix I, Germain H. Custom selected reference genes outperform pre-defined reference genes in transcriptomic analysis. BMC Genomics. 2020 Jan. 10; 21(1):35, Zhang B, Wu X, Liu J, Song L, Song Q, Wang L, Yuan D, Wu Z. β-Actin: Not a Suitable Internal Control of Hepatic Fibrosis Caused by Schistosoma japonicum. Front Microbiol. 2019 Jan. 31; 10:66, Veres-Szekely A, Pap D, Sziksz E, Jivorszky E, Rokonay R, Lippai R, Tory K, Fekete A, Tulassay T, Székely, Vannay Á. Selective measurement of a smooth muscle actin: why β-actin cannot be used as a housekeeping gene when tissue fibrosis occurs. BMC Mol Biol. 2017 Apr. 27; 18(1):12, and Wisniewski J R, Mann M. A Proteomics Approach to the Protein Normalization Problem: Selection of Unvarying Proteins for MS-Based Proteomics and Western Blotting. J Proteome Res. 2016 Jul. 1; 15(7):2321-6, the contents of which are incorporated by reference herein, in their entireties, for all purposes.

In some embodiments of a computationally-derived “housekeeper” marker method, the normalized profiles are defined as follows: Qᵢₛ′= [symbol] / [symbol] , where [symbol] is the original abundance level (e.g. expression level amount detected) of a marker i in a sample s, and [symbol] is an abundance level of a housekeeper marker in a sample s. In this manner, it is possible to search for a “computationally-derived housekeeper” by testing as all candidate housekeepers (with non-zero abundance levels in all samples) and determine the one, which makes possible the most accurate classification.

Alternatively in some embodiments, a biomarker is defined as a comparison, e.g., ratio, of expression values: Qᵢⱼₛ′= [symbol] / [symbol] ). This approach implies that the biological invariants (and differences) are determined by ratios of biological features rather than by absolute values of the features. In this iteration the biological features are molecular signals, which can include but are not limited to gene expression levels, protein abundance, epigenetic and posttranslational modifications, etc. This also means that the essential biological differences are more strongly associated with molecular signal ratios rather than with the absolute values of signals.

In support of this second iteration, biomarkers as ratios of expression values, we introduced and tested “pairwise biomarkers” defined as the differences between logarithms of abundance levels of all pairs of autoantibodies (AAbs). While this example uses AAbs, we believe any dataset wherein differences between pairs can be defined, proteomic (mass spectroscopy data, proteins, peptide fragments), genomic (RNA expression levels, microbiome data), etc. can be so converted.

Thus, and in the examples provided below, for M antibodies and, respectively, M*(M−1)/2 unique pairs of antibodies, the differences between logs of abundance levels in each of the samples were computed and those pairwise differences were themselves used as biomarkers. Because the total number of unique pairs in autoantibody profiles is large ˜15*10⁶, some statistically significant associations can be produced by random rather than by true underlying biological associations. To control for the possibility of random associations, in some embodiments, additional tests are performed with randomized distributions of diagnosis labels in sample cohorts to assess probabilities of random occurrence of statistically significant associations between pairwise biomarkers and diagnoses. Based on this test, in some embodiments, a P value threshold (Mann-Whitney-Wilcoxon test) is determined to sort out non-diagnosis related pairwise biomarkers produced by random. For instance, in some of the examples provided below, the results were obtained using statistical thresholds set at Pv<10⁻⁶⁻⁷, which excludes or minimizes random associations between pairwise biomarkers and diagnoses.

Advantageously, the statistical differentiation between AAB profiles of patients of different diagnoses increases when pairwise biomarkers—ratios of logs of AAB abundances are used. Further, using pairwise biomarkers makes possible classification of AAB profiles with clinically relevant accuracy.

Example Feature Selection and Classifier Training Methodology

In some embodiments, the methods described herein rely upon a two-step computational protocol, including (i) use of a statistical algorithm for determining candidate features that are associated with pathway-specific genomic alterations and (ii) use of a machine learning algorithm for determining the optimal weights of combinations of candidate features to derive scoring functions—a signature for predicting key driver alterations in major cancer pathways. One embodiment of this process is described in Rykunov et al. et al. 2016 Nuc Acids Res 44(11), e110, which is incorporated herein by reference, in its entirety, for all purposes.

In some embodiments, the methods include selecting a ranked list of biomarkers by (1) defining a list of biomarkers, e.g., pairwise biomarkers as a difference between logarithms of given molecular signals (e.g. gene expression levels, protein abundances, etc. . . . ), and (2) using a boosting technique to rank the biomarkers, e.g., pairwise biomarkers. In order to boost, an original data set is repeatedly divided by random into, e.g., equal, training and test sets, and biomarkers, e.g., pairwise biomarkers, differentially distributed between two classes in both sets are been identified and ranked both by statistical power (P value) and by occurrence. For more information on this boosting technique see, for example, Rykunov et al. et al. 2016 Nuc Acids Res 44(11), e110.

Next, a classifier is identified by running classification tests and determining the optimal classification signature. In some embodiments, the algorithm takes as input a ranked list of candidate biomarkers (e.g., from steps 1 and 2, described above) and a dataset of molecular profiles. All possible sets of biomarkers are been tested by adding biomarkers singly and in succession. For each of the biomarker sets (typically, from 2 to 35) a dataset of molecular profiles is divided into two classes (e.g. cancer/benign, or Polyps/no Polyps). A classification function that optimizes the separation between given diagnostic classes is then computed as a weighted sum of biomarker levels, where weights are computed analytically using correlations between pairs of selected biomarkers. The training set is used to determine biomarker weights and optimal classification thresholds to be tested in the independent test set. For each samples of test set, the scoring function is computed using sample biomarker's values and weights determined in training set; then classifications is made based on the threshold of training set. The overall accuracy of classification is assess in multiple classification tests where half of a given dataset is used as training set and another half is used as test set. Thus, for each set of a ranked list of candidate biomarkers and each samples, the probability of correct classification and average scoring were computed in multiple classification tests. These values were then used for computation of overall classification accuracies assessed by area under receiver operating curve (AUC) both for averaged classification scores and for probabilities. Based on the obtained AUC values, the final list of biomarkers, their weights, and classification threshold is determined. For more information on this classifier identification technique see, for example, Rykunov et al. et al. 2016 Nuc Acids Res 44(11), e110.

Evaluating a Subject for a Stage of Endometrial or Ovarian Cancer

Referring to block 202 of FIG. 2, a method for evaluating a subject for a stage of a disease condition. In some embodiments, the method evaluates a subject for a stage of endometrial cancer. In some embodiments, the method evaluates a subject for a stage of ovarian cancer.

In some embodiments, the method evaluates a subject for a disease condition. In some such embodiments, the disease condition comprises a non-cancerous condition. In some embodiments, the non-cancerous condition is endometriosis, tuberculosis, fungal infections, or bacterial pneumonias. See Radha et al. et al. 2014 J Cytol. 31(3), 136-138. In some embodiments, the non-cancerous condition is pericoronitis, hematemesis, ulcerative colitis, ulcer, osteoarthritis, sinusitis, or other conditions known in the art.

In some such embodiments, the disease condition comprises a pre-cancerous or cancer condition. A pre-cancerous disease condition involves abnormal cells that are at an increased risk of developing into cancer. In some embodiments, the cancer condition comprises endometrial cancer, ovarian cancer, cervical cancer, uterine sarcoma, vaginal cancer, vulvar cancer, gestational trophoblastic disease, or other reproductive cancer. In some embodiments, the cancer condition comprises breast cancer, esophageal cancer, lung cancer, renal cancer, colorectal cancer, nasopharyngeal cancer, lymphoma, or any other cancer condition known in the art.

In some embodiments, the stage of endometrial cancer comprises stage 0 endometrial cancer (e.g., complex atypical hyperplasia), stage IA endometrial cancer, stage IB endometrial cancer, stage II endometrial cancer, stage III endometrial cancer, or stage IV endometrial cancer. In some embodiments, the stage of ovarian cancer comprises stage 0 ovarian cancer, stage IA ovarian cancer, stage IB ovarian cancer, stage II ovarian cancer, stage III ovarian cancer, or stage IV ovarian cancer.

In some embodiments, the subject is asymptomatic for endometrial cancer. In some embodiments, the subject is asymptomatic for ovarian and/or endometrial cancer. In some embodiments, subjects are asymptomatic for endometrial cancer but do exhibit complex atypical hyperplasia (CAH). This is a pre-cancerous state (e.g., equivalent to stage 0 endometrial cancer) that is associated with an approximately 40% increased risk of a subject developing endometrial cancer. See e.g., Suh-Burgmann et al. et al. 2009 Obstetrics and Gynecology 114(3), 523-529. In some embodiments, the subject is symptomatic for ovarian and/or endometrial cancer. In some embodiments, a subject is from a population with an increased risk for ovarian and/or endometrial cancer. In some embodiments, the increased risk is that the subject has Lynch syndrome, the subject is obese, the subject has family history of ovarian and/or endometrial cancer, the subject has a BRCA mutation, and/or the subject is over a predetermined age—e.g., where the predetermined age is at least 40, at least 45, at least 50, at least 55, at least 60, at least 65, or at least 70 years of age).

In some embodiments, a subject is concurrently evaluated for a stage of an additional cancer condition distinct from ovarian and endometrial cancer. In some embodiments, another cancer condition is selected from the group consisting of lung cancer, prostate cancer, colorectal cancer, renal cancer, cancer of the esophagus, cervical cancer, bladder cancer, gastric cancer, nasopharyngeal cancer, or a combination thereof.

Referring to block 204, the evaluation method proceeds by obtaining a biological sample from the subject. In some embodiments, the biological sample of the subject is a lavage fluid sample.

In some embodiments, the lavage fluid sample is a uterine lavage fluid sample. In some embodiments, uterine lavage fluid is collected from the subject via hysteroscopy combined with curettage. In some embodiments, uterine lavage fluid is collected from the subject via uterine washings. In some embodiments, the lavage fluid sample is a bronchoalveolar lavage fluid sample, a gastric lavage fluid sample, a ductal lavage fluid sample, a nasal irrigation sample, a peritoneal lavage fluid sample, a peritoneal lavage fluid sample, an arthroscopic lavage fluid sample, or ear lavage fluid sample.

In some embodiments, a body cavity from which the lavage fluid sample is collected determines which type(s) of cancer said lavage fluid sample is assayed for (e.g., bladder cancer, oral cancer, lung cancer, gastrointestinal cancer, endometrial, and/or ovarian). In some such embodiments, the method further evaluates the subject for a stage of bladder cancer, a stage of oral cancer, a stage of lung cancer, a stage of gastrointestinal cancer, a stage of endometrial cancer, and/or a stage of ovarian cancer, respectively.

Referring to block 206, the evaluation method continues by analyzing the lavage fluid sample through a proteomics analysis for an abundance of each autoantibody in a plurality of autoantibodies, using a respective protein for each autoantibody in the plurality of autoantibodies. Through the proteomics analysis, an autoantibody abundance dataset of the subject is obtained. The autoantibody abundance dataset includes a respective abundance of each autoantibody in the plurality of autoantibodies.

In some embodiments, the proteomics analysis comprises obtaining IgG and IgA profiles of the plurality of autoantibodies obtained from the lavage fluid sample (e.g., the biological sample). In some embodiments, the IgG and IgA profiles are combined, thereby determining the respective abundance level of each autoantibody in the plurality of autoantibodies. In some embodiments, only one of either of the IgG or IgA profiles is used.

Referring to block 208, the evaluation method proceeds with filtering the autoantibody abundance dataset in accordance with a set of reference features. The filtering results in a set of targeted autoantibody abundance levels for the subject.

In some embodiments, one or more reference features may be selected from a list of predicted molecular pathways and/or cell type signatures in Table 1 (e.g., predicted molecular pathways and/or cell type signatures that are known to be differentially regulated—e.g., up- or downregulated—in cancer subjects). The molecular pathways and/or cell type signatures in Table 1 are collected from one or more publicly curated datasets. See e.g., Kanehisa et al. et al. 2019 Nuc Acids Res 47, D590-D595; Fabregat et al. et al. 2018 Nuc Acids Res 46, D649-D655; Aran et al. et al. 2017 Genome Biol 18, 220; and Targonski et al. et al. 2019 Sci Reports 9, 9747.

TABLE 1
Molecular Pathways and/or Cell Type Signatures

| | | Fold change |
|---|---|---|
| | | in cancer vs |
| | | healthy |
| Molecular Pathway and/or Cell Type Signature | Database | individuals |
| B-Catenin-WNT_Signaling_{——}xccpw | | 1.83 |
| Transcriptional activity of SMAD2/SMAD3:SMAD4 | Reactome | 1.81 |
| heterotrimer | | |
| Cell-extracellular matrix interactions | Reactome | 1.75 |
| naiveB-cells_NOVERSHTERN_1 | xCell | 1.73 |
| SMAD2/SMAD3:SMAD4 heterotrimer regulates transcription | Reactome | 1.72 |
| Alpha-defensins | Reactome | 1.71 |
| Lysosome | KEGG | 1.52 |
| AKT phosphorylates targets in the nucleus | Reactome | −1.44 |
| Free fatty acids regulate insulin secretion | Reactome | −1.45 |
| Fatty Acids bound to GPR40 (FFAR1) regulate insulin secretion | Reactome | −1.45 |
| Acetylcholine regulates insulin secretion | Reactome | −1.48 |
| Mitochondrial iron-sulfur cluster biogenesis | Reactome | −1.48 |
| Mitochondrial Fatty Acid Beta-Oxidation | Reactome | −1.49 |
| ERCC6 (CSB) and EHMT2 (G9a) positively regulate rRNA | Reactome | −1.50 |
| expression | | |
| Degradation of DVL | Reactome | −1.51 |
| CoenzymeA biosynthesis | Reactome | −1.51 |
| CD8 + T-cells_BLUEPRINT_1 | | −1.52 |
| Gene Silencing by RNA | Reactome | −1.52 |
| CD8 + T-cells_IRIS_3 | xCell | −1.53 |
| Glycolysis Can Res_{——}CancerResearch | | −1.54 |
| Fatty acid elongation | KEGG | −1.54 |
| Gene and protein expression by JAK-STAT signaling after | Reactome | −1.54 |
| Interleukin-12 stimulation | | |
| Association of TriC/CCT with target proteins during biosynthesis | Reactome | −1.54 |
| Hh mutants abrogate ligand secretion | Reactome | −1.55 |
| ClassC/3 (Metabotropic glutamate/pheromone ereceptors) | Reactome | −1.56 |
| Classical Kir channels | Reactome | −1.56 |
| MET interacts with TNS proteins | Reactome | −1.57 |
| N-Glycan antennae elongation | Reactome | −1.57 |
| Vif-mediated degradation of APOBEC3G | Reactome | −1.57 |
| Regulation of DNA replication | Reactome | −1.58 |
| Receptor_Tyrosine_KinaseORGrowth_Factor_Signaling_{——}xccpw | | −1.58 |
| Defective CFTR causes cystic fibrosis | Reactome | −1.60 |
| Synthesis of PS | Reactome | −1.61 |
| Theretinoid cycle in cones (daylight vision) | Reactome | −1.61 |
| M/G1 Transition | Reactome | −1.62 |
| DNA Replication Pre-Initiation | Reactome | −1.62 |
| RHO GTPases Activate WASPs and WAVEs | Reactome | −1.63 |
| INTERFERON_ALPHA_RESPONSE | Hallmark | −1.64 |
| Post-translational modification: synthesis of GPI-anchored | Reactome | −1.64 |
| proteins | | |
| naiveB-cells_HPCA_1 | xCell | −1.64 |
| MEP_HPCA_1 | xCell | −1.65 |
| Activation and oligomerization of BAK protein | Reactome | −1.67 |
| Macrophages M1 BLUEPRINT_2 | xCell | −1.68 |
| Interleukin-1 family signaling | Reactome | −1.68 |
| Signaling by the BCell Receptor (BCR) | Reactome | −1.71 |
| Aminoacyl-tRNA biosynthesis | KEGG | −1.72 |
| Interferon alpha/beta signaling | Reactome | −1.73 |
| Regulation of mRNA stability by proteins that bind AU-rich | Reactome | −1.73 |
| elements | | |
| Cytokine-cytokine receptor interaction | KEGG | −1.73 |
| Glycolysis/Gluconeogenesis | KEGG | −1.77 |
| Infectious disease | Reactome | −1.79 |
| Dectin-1 mediated noncanonical NF-kB signaling | Reactome | −1.79 |
| HIV Infection | Reactome | −1.80 |
| Toll Like Receptor 3 (TLR3) Cascade | Reactome | −1.80 |
| Protein folding | Reactome | −1.81 |
| Preadipocytes ENCODE 3 | xCell | −1.83 |
| MYC TARGETS V1 | Hallmark | −1.85 |
| NOTCH SIGNALING | Hallmark | −1.86 |
| tRNA Aminoacylation | Reactome | −1.87 |
| Myocytes ENCODE3 | xCell | −1.87 |
| Smooth muscle HPCA3 | xCell | −1.87 |
| Metabolism of polyamines | Reactome | −1.88 |
| TRIF (TICAM1)-mediated TLR4 signaling | Reactome | −1.88 |
| MyD88-independent TLR4 cascade | Reactome | −1.88 |
| Toll-Like Receptors Cascades | Reactome | −1.89 |
| Chaperonin-mediated protein folding | Reactome | −1.89 |
| Signaling by NOTCH1 | Reactome | −1.89 |
| Activated TLR4 signaling | Reactome | −1.93 |
| Host Interactions of HIVf actors | Reactome | −1.95 |
| Formation of TC-NER Pre-Incision Complex | Reactome | −1.97 |
| Cytosolic tRNA aminoacylation | Reactome | −2.03 |
| Activated NOTCH1 Transmits Signal to the Nucleus | Reactome | −2.03 |
| Toll Like Receptor4 (TLR4) Cascade | Reactome | −2.04 |

Referring to block 210, the evaluation method inputs the set of targeted autoantibody abundance levels into a trained classifier. The trained classifier provides a probability or likelihood that the subject has a disease condition, e.g., a stage of endometrial or ovarian cancer.

In some embodiments, the trained classifier provides a probability or likelihood that the subject has each respective stage of endometrial or ovarian cancer (e.g., to provide information as to which stage of endometrial or ovarian cancer the subject most likely has).

In some embodiments, the trained classifier comprises a machine learning algorithm, molecular signature algorithm, a neural network algorithm, a support vector machine algorithm, a decision tree algorithm, an unsupervised clustering model algorithm, a supervised clustering model algorithm, or a regression model. In preferred embodiments, the trained classifier comprises a molecular signature (MSM) algorithm trained in accordance with the methods described in block 310. See Rykunov et al. et al. 2016 Nuc Acids Res 44(11), e110.

In some embodiments, the obtaining further comprises extracting a plurality of nucleic acid sequence reads from a lavage fluid sample (e.g., or from a biological sample). In some embodiments, the analyzing further comprises sequencing the plurality of nucleic acid sequence reads targeted by a panel of genes with a predetermined minimum coverage value (e.g., ultra-deep sequencing), thereby obtaining a set of gene expression levels for the subject. In some embodiments, the inputting further comprises inputting the set of gene expression levels.

In some embodiments, the panel of genes comprises at least 2 genes, at least 5 genes, at least 10 genes, at least 15 genes, or at least 20 genes. In some embodiments, the panel of genes (e.g., genes from a list of predicted molecular pathways and/or cell type signatures) is selected from Table 1.

In some embodiments of the present disclosure, the method comprises obtaining (a) a biological sample from the subject, and analyzing (b) the biological sample for an abundance, E, of each autoantibody in a plurality of autoantibodies, thereby obtaining an autoantibody abundance dataset for the subject that includes an abundance of each autoantibody in the plurality of autoantibodies.

In some embodiments, each autoantibody in the plurality of autoantibodies corresponds to an autoantibody; and analyzing the biological sample comprises performing a proteomics analysis that includes using a protein for each autoantibody in the plurality of autoantibodies.

The method continues with filtering (c) the autoantibody abundance dataset in accordance with a set of reference features, thereby obtaining a set of targeted autoantibody abundance levels for the subject. In some embodiments, filtering the autoantibody abundance dataset includes applying the overall ranked set of autoantibodies to a feature extraction method.

The method further includes determining (d), at least in part based on the set of targeted autoantibody abundance levels, a disease profile for the subject.

In some embodiments, the disease profile is obtained in accordance with methods described in Rykunov et al. et al. 2016 Nuc Acids Res 44(11), e110. In some embodiments, the disease profile Vₛ for the tumor s is calculated as:

Vₛ=ΣₘAₘ·Eₘₛ.

In such embodiments, m is an autoantibody, Aₘ is a weight for autoantibody m, and Eₘₛ is an expression level of each autoantibody in tumor s.

In some embodiments, the weight for each autoantibody, Aₘ, is calculated as:

$${A}_{m} ∼ {D}_{m}^{- 1} {\sum }_{k} {⌊ {C}_{m k} ⌋}^{- 1} {Z}_{k} .$$

In such embodiments, Dₘ is the standard deviation of expression of the autoantibody m, k is a second autoantibody, [Cₘₖ] is matrix of pairwise correlations between expression of autoantibodies m and k, and Zₖ is a z-score for second autoantibody k.

In some embodiments, van element Cₘₖ is calculated as:

$${C}_{m k} = \frac{{\sum }_{s} ( {E}_{m s} - 〈 {E}_{m} 〉 ) ( {E}_{k s} - 〈 {E}_{k} 〉 )}{{D}_{m} {D}_{k}}$$

[Cₘₖ]⁻¹ an element of the inverse matrix; (E)ₘ and Dₘ the average expression and standard deviation, respectively, of the expression for candidate autoantibody m; S the total number of tumors in a data set.

In some embodiments, Zₖ is calculated as:

$${Z}_{k} = \frac{{〈 {E}_{k} 〉}_{1} - {〈 {E}_{k} 〉}_{2}}{{D}_{k}}$$

In such embodiments, [symbol] Eₘ [symbol] is the average expression each autoantibody m, and [symbol] Eₖ [symbol] ₁ and [symbol] Eₖ [symbol] ₂ are the average expression levels for second autoantibody k computed for data classes 1 (non-altered pathways) and 2 (altered pathways), respectively.

The method proceeds by applying (e) the disease profile to a trained classifier, thereby obtaining a probability or likelihood from the trained classifier that the subject has the disease condition.

Classification Method

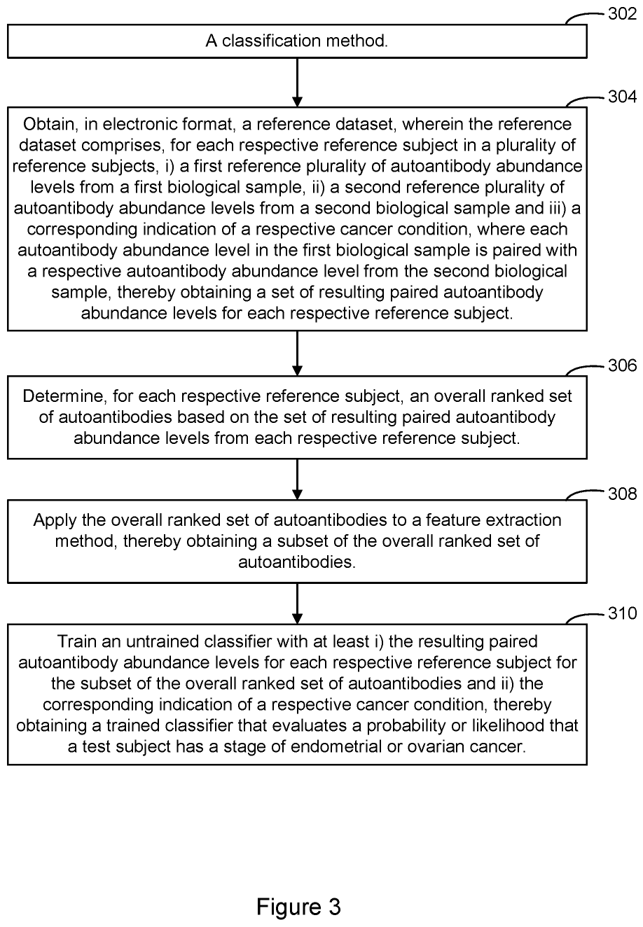

Referring to block 302 in FIG. 3, a classification method is provided. To reduce the effect of systematic errors (e.g. batch effects), biomarkers were analyzed. In some embodiments, the biomarkers are defined as the differences between logarithms of abundance levels of all pairs of autoantibodies. In some embodiments, any dataset wherein differences between pairs can be defined, proteomic, genomic, etc. can be used as biomarkers. In some embodiments, for N antibodies and, respectively, N*(N−1)/2 unique pairs of antibodies, the differences between logs of abundance levels in each of the samples were computed and those pairwise differences were themselves used as biomarkers. In some embodiments, because the total number of unique pairs is large ˜15*10⁶, some statistically significant associations can be produced by random rather than by true underlying biological associations. To control for the possibility of random associations, additional tests are performed in some embodiments, with randomized distributions of diagnosis labels in sample cohorts to assess probabilities of random occurrence of statistically significant associations between pairwise biomarkers and diagnoses. Based on this test, in some embodiments, a P value threshold (Mann-Whitney U test) is used to sort out non-diagnosis related pairwise biomarkers produced by random. In some embodiments, the results were obtained using statistical thresholds set at P<10⁻⁶⁻⁷, which exclude or minimize random associations between pairwise biomarkers and diagnoses.

Referring to block 304, the classification method proceeds by obtaining a reference dataset. The reference dataset comprises, for each respective reference subject in a plurality of reference subjects, a i) a first reference plurality of autoantibody abundance levels from a respective first biological sample, ii) a second reference plurality of autoantibody abundance levels from a respective second biological sample, and iii) a respective disease condition. Each autoantibody abundance level in the first biological sample is paired with an autoantibody abundance level from the second biological sample, thereby obtaining a set of resulting paired autoantibody abundance levels for each respective reference subject.

In some embodiments, each respective first biological sample comprises a lavage fluid sample comprising uterine lavage fluid, bladder lavage fluid, oral rinse, or lung washings. In some embodiments, each respective first biological sample comprises another type of biological sample (e.g., such as blood, plasma, serum, urine, cerebrospinal fluid, fecal, saliva, sweat, tears, pleural fluid, pericardial fluid, or peritoneal fluid of the respective subject). In some embodiments, uterine lavage fluid is collected from the subject via hysteroscopy combined with curettage. In some embodiments, uterine lavage fluid is collected from the subject via uterine washings. In some embodiments, the body cavity from which the lavage fluid was collected determines which type(s) of cancer said lavage fluid will be assayed for. For example, lavage fluid collected from the urethra can be used to evaluate a subject for bladder cancer; lavage fluid collected from the mouth or throat can be used to evaluate a subject for oral cancer; lavage fluid collected from the lungs can be used to evaluate a subject for lung cancer; or lavage fluid collected from the stomach and/or intestines can be used to evaluate a subject for gastrointestinal cancer. In some embodiments, the lavage fluid sample is collected from a subject during an annual exam or other screening (e.g., concurrent with a pap or STD test).

In some embodiments, each second biological sample (e.g., a control sample for the respective subject that reflects non-cancerous autoantibody levels) comprises a serum sample from the respective subject. In some embodiments, each second biological sample comprises blood, plasma, serum, urine, cerebrospinal fluid, fecal, saliva, sweat, tears, pleural fluid, pericardial fluid, or peritoneal fluid of the respective subject.

In some embodiments, the respective cancer condition of each reference subject in a first set of the reference subjects in the plurality of reference subjects comprises non-cancer (e.g., a healthy control population).

In some embodiments, the respective cancer condition of each reference subject in a second set of the plurality of reference subjects comprises stage 0 endometrial cancer, stage IA endometrial cancer, stage IB endometrial cancer, stage II endometrial cancer, stage III endometrial cancer, or stage IV endometrial cancer. In some embodiments, the respective cancer condition of each reference subject in the second set of the plurality of reference subjects comprises stage 0 ovarian cancer, stage IA ovarian cancer, stage IB ovarian cancer, stage II ovarian cancer, stage III ovarian cancer, or stage IV ovarian cancer.

In some embodiments, the respective cancer condition of each reference subject in the second set of the plurality of reference subjects is selected from the group consisting of lung cancer, prostate cancer, colorectal cancer, renal cancer, cancer of the esophagus, cervical cancer, bladder cancer, gastric cancer, or nasopharyngeal cancer.

Referring to block 306, the classification method continues by determining, for each respective reference subject, an overall ranked set of autoantibodies based on the set of resulting paired autoantibody abundance levels from each respective reference subject.

For example, for each reference subject, each autoantibody abundance from the respective first biological sample is compared to the corresponding autoantibody abundance from the corresponding paired second biological sample (e.g., comparing in autoantibody abundance from the uterine lavage fluid collected from the respective subject—e.g., abundance levels that may be due to ovarian or endometrial cancer—to the corresponding autoantibody abundance from the second biological sample collected from the respective subject—e.g., background, non-cancer related abundance levels). Thus, for each reference subject, a respective overall ranked set of autoantibodies is obtained.

Referring to block 308, the classification method applies the overall ranked set of autoantibodies to a feature extraction method. A subset of the overall ranked set of autoantibodies is obtained from the feature extraction method.

In some embodiments, the subset of the overall ranked set of autoantibodies corresponds to a list of predicted molecular pathways and/or cell type signatures in Table 1.

In some embodiments, obtaining the subset of the overall ranked set of autoantibodies includes removing from the ranked set of autoantibodies one or more autoantibodies that do not meet a first criterion. In some embodiments, the first criterion includes a p-value threshold, where ranked autoantibodies with p-values higher than the p-value threshold are removed. In some embodiments, obtaining the subset of overall ranked set of autoantibodies includes applying a feature extraction method to the overall ranked set of autoantibodies. In some embodiments, the feature extraction method uses Fisher's exact test, t-test, or other test to determine p-values (e.g., for comparison to the p-value threshold) for each autoantibody in the ranked set of autoantibodies. See e.g., Fodor 2002 Center for Applied Scientific Computing, Lawrence Livermore National, Technical Report UCRL-ID-148494 and Cunningham 2007 University College Dublin, Technical Report UCD-CSI-2007-7, each of which are hereby incorporated by reference.

Referring to block 310, the classification method trains an untrained classifier using at least: i) the resulting paired autoantibody abundance levels for each respective reference subject for the subset of the overall ranked set of autoantibodies, and ii) the corresponding indication of a respective disease condition. A trained classifier that evaluates a probability or likelihood that a test subject has a disease condition, e.g., a stage of endometrial or ovarian cancer, is thereby obtained.

The trained classifier obtained therein can be used in accordance with methods described in blocks 202-210 above. As described above, many types of classifiers can be used in conjunction with the methods described herein.

In one embodiment, an example evaluation method may include obtaining one or more biological samples of a subject. A first biological sample may be a uterine lavage fluid. The example method may analyze the first biological sample for levels of abundance of a set of autoantibodies through one or more proteomics analyses. A second biological sample may be another type of fluid sample such as the blood sample of the subject. The example method may analyze the second biological sample for levels of abundance of a set of autoantibodies through one or more proteomics analyses. The results of obtained from the first biological sample and the second biological sample for the abundance level of the same autoantibody may be cross-referenced (e.g., aggregated, compared, selected) or may be treated independently. A third biological sample may be yet another fluid or tissue of the subject for nucleotide acid sequencing. The gene expression levels for the subject may be determined by the sequences. Alleles at certain targeted loci of single nucleotide polymorphism (SNP) may also be assayed to generate a genetic dataset of the individual. In one embodiment, one or more biological sample may be repeatedly used for different analyses. For example, a blood sample may be used to obtain autoantibody abundance levels and be used for DNA sequencing.

The example method may also select one or more targeted autoantibody abundance levels for the subject. The selection may be based on a set of reference molecular pathways and/or cell-type signatures. The example method may also select genetic data values related to targeted gene loci that are associated with the set of reference molecular pathways and/or cell-type signatures. The example method may obtain additional data on the subject. For example, the method may obtain disease condition-relevant morphometric data of the subject. The disease condition may be endometrial cancer or ovarian cancer. The morphometric data may include age, history of pregnancy, history of breastfeeding, BRCA1 genotype, BRCA2 genotype, history of breast cancer, family history of endometrial cancer, ovarian cancer, or breast cancer.

The method may further include one or more measurements (e.g., targeted autoantibody abundance levels) and other data of the subject into a set of numerical values that may be used as an input of a machine learning algorithm. For example, the set of numerical values may be represented as an N-dimensional vector. In one embodiment, the set of numerical values may be referred to as disease profile Vₛ. The disease profile may be represented by the equation Vₛ=ΣₘAₘ·Eₘₛ, but in other embodiments the disease profile may be represented differently. For example, each value in the set may represent a measurement or a trait of the individual. The value may be scaled or normalized to bring the values in the set to a similar order of magnitude. For measurements such as targeted autoantibody abundance levels, the measurement value may be used directly as one of the numerical values. The measurement value may also be mapped to another value based on one or more formulas (e.g., linear scaling or non-linear mapping). For traits such as genotypes, phenotypes, medical records of the subject that may not be naturally represented by a number, the trait may be converted to a number or a scale. For example, a presence or absence of a phenotype may be represented by a binary number. A dominant allele or a recessive allele may also be represented by a binary number. Some traits may be represented by a scale. The trait represented by a number may likewise be mapped to another value based on one or more formulas. Other features are also possible. For example, the features can be any suitable values that can be used in differentiating samples—demographic characteristics (e.g. Age, BMI, . . . ), results of blood test, individual antibody abundances; average abundances of proteins representing molecular pathways from different pathway database; assessments of activities of molecular pathways; scoring functions derived from subnetworks of proteins and many other things which can used. Any quantitative assessments that can be deduced from antibody abundances. These numerical assessments may be treated as features. In one embodiment, the set of numerical values may include only measurements of the targeted autoantibody abundance levels that are obtained from the uterine lavage sample. In another embodiment, the set of numerical values may additionally include measurements of the targeted autoantibody abundance levels that are obtained from the second biological sample. In yet another embodiment, the set of numerical values may further include values derived from other sources such as the subject's genotype data, morphometric data, and other suitable identifiable traits.

The method may input the set of numerical values into a machine learning algorithm to determine a prediction. The output of the machine learning algorithm may be a prediction of whether the subject has a disease, such as endometrial cancer, ovarian cancer, or breast cancer. Predictions of other diseases may also be possible in other embodiments. The use of measurements of autoantibody abundance levels to predict diseases is not limited to only predicting a certain type of cancer. Also, the prediction may take various forms, depending on the machine learning algorithm. For example, the prediction may be a probability or likelihood that the subject has a disease condition. The prediction may also be a classification, such as a binary classification predicting the subject has a disease condition or does not have the disease condition, or multi-class output predicting what kinds of diseases the subject may have among a selection of diseases (e.g., a selection of various types of cancer).

In various embodiments, a wide variety of machine learning techniques may be used. Examples of which include different forms of unsupervised learning, clustering, supervised learning such as random forest classifiers, support vector machine (SVM) such as kernel SVMs, gradient boosting, linear regression, logistic regression, and other forms of regressions. Deep learning techniques such as neural networks, including recurrent neural networks (RNN) and long short-term memory networks (LSTM), may also be used. Customized machine learning techniques, such as molecular signature model (MSM), may also be used.

In a certain embodiment, a machine learning model may include certain layers, nodes, and/or coefficients. The machine learning model may be associated with an objective function, which generates a metric value that describes the objective goal of the training process. For example, the training may intend to reduce the error rate of the model by reducing the output value of the objective function, which may be called a loss function. Other forms of objective functions may also be used, particularly for unsupervised learning models whose error rates are not easily determined due to the lack of labels.

In one embodiment, a supervised learning technique is used. Patients with known disease conditions may be classified into two groups, which may be referred to as a positive training set (patients with the disease condition) and a negative training set (patients without the disease condition). In some supervised learning techniques, the objective function of the machine learning algorithm may be the training error rate in predicting the patients in the two training sets. For example, the objective function may be cross-entropy loss. In another embodiment, an unsupervised learning technique is used and the patients used in training are not labeled with disease condition. Various unsupervised learning technique such as clustering may be used. In yet another embodiment, the machine learning model may be semi-supervised.

Taking an example of a neural network as the machine learning model, training of the CNN may include forward propagation and backpropagation. A neural network may include an input layer, an output layer, and one or more intermediate layers that may be referred to as hidden layers. Each layer may include one or more nodes, which may be fully or partially connected to other nodes in adjacent layers. In forward propagation, the neural network performs computation in the forward direction based on outputs of a preceding layer. The operation of a node may be defined by one or more functions. The functions that define the operation of a node may include various computation operations such as convolution of data with one or more kernels, recurrent loop in RNN, various gates in LSTM, etc. The functions may also include an activation function that adjusts the weight of the output of the node. Nodes in different layers may be associated with different functions.

Each of the functions in a machine learning model may be associated with different coefficients that are adjustable during training. In addition, some of the nodes in a neural network each may also be associated with an activation function that decides the weight of the output of the node in forward propagation. Common activation functions may include step functions, linear functions, sigmoid functions, hyperbolic tangent functions (tanh), and rectified linear unit functions (ReLU). The data of a patient in the training set may be converted to a feature vector in a manner described above. After a feature vector is inputted into the neural network and passes through a neural network in the forward propagation, the results may be compared to the training label of the patient to determine the neural network's performance. The process of prediction may be repeated for other patients in the training sets to compute the value of the objective function in a particular training round. In turn, the neural network performs backpropagation by using coordinate descent such as stochastic coordinate descent (SGD) to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. A trained model may be used to predict the disease condition of a new subject.

While the training is described using a neural network as an example, a similar training process may be used for other suitable machine learning algorithms. In training a machine learning algorithm, various regularization techniques and cross-validation techniques may be used to reduce the chance of over-fitting the algorithm.

Evaluating a Subject for a State of a Gynecologic Disorder

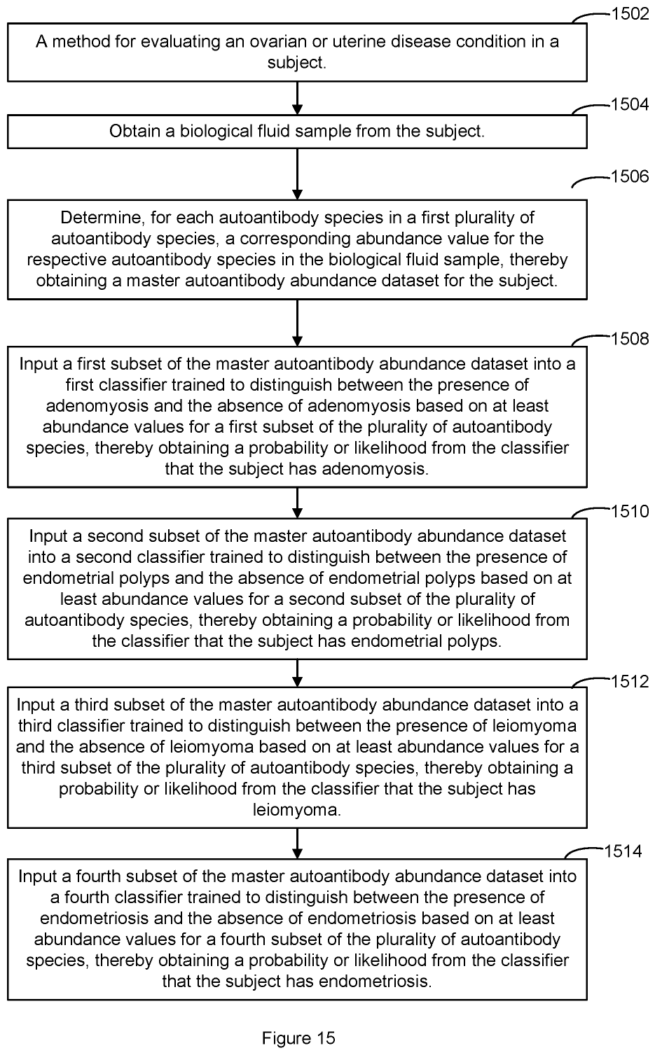

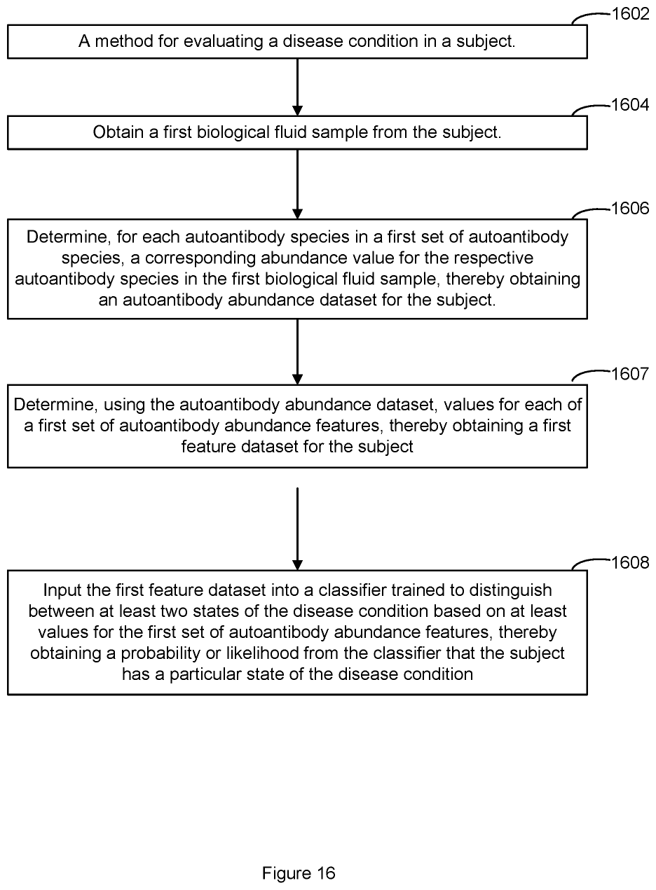

FIGS. 14 and 15 illustrate example methods 1400 and 1500 for evaluating a gynecological disorder (also referred to herein as an ovarian or uterine disease) in a subject using autoantibody biomarkers found in a biological fluid sample, e.g., a blood plasma or uterine lavage fluid, from the subject.

Referring to block 1402 of FIG. 14, a method is provided for evaluating an ovarian or uterine disease condition in a subject. In some embodiments, the ovarian or uterine disease condition is an ovarian cancer or an endometrial cancer. In some embodiments, the ovarian or uterine disease condition is adenomyosis, endometrial polyps, leiomyoma, or endometriosis (e.g., complex atypical hyperplasia and/or an atrophic endometrium and/or an endometrial thickening).

In some embodiments, the method evaluates a subject for a disease condition. In some such embodiments, the disease condition comprises a non-cancerous condition. In some embodiments, the non-cancerous condition is endometriosis, tuberculosis, fungal infections, or bacterial pneumonias. See Radha et al. et al. 2014 J Cytol. 31(3), 136-138. In some embodiments, the non-cancerous condition is pericoronitis, hematemesis, ulcerative colitis, ulcer, osteoarthritis, sinusitis, or other conditions known in the art.

In some such embodiments, the disease condition comprises a pre-cancerous or cancer condition. A pre-cancerous disease condition involves abnormal cells that are at an increased risk of developing into cancer. In some embodiments, the cancer condition comprises endometrial cancer, ovarian cancer, cervical cancer, uterine sarcoma, vaginal cancer, vulvar cancer, gestational trophoblastic disease, or other reproductive cancer. In some embodiments, the cancer condition comprises breast cancer, esophageal cancer, lung cancer, renal cancer, colorectal cancer, nasopharyngeal cancer, lymphoma, or any other cancer condition known in the art.

In some embodiments, the stage of endometrial cancer comprises stage 0 endometrial cancer (e.g., complex atypical hyperplasia), stage IA endometrial cancer, stage IB endometrial cancer, stage II endometrial cancer, stage III endometrial cancer, or stage IV endometrial cancer. In some embodiments, the stage of ovarian cancer comprises stage 0 ovarian cancer, stage IA ovarian cancer, stage IB ovarian cancer, stage II ovarian cancer, stage III ovarian cancer, or stage IV ovarian cancer.

In some embodiments, the subject is asymptomatic for endometrial cancer. In some embodiments, the subject is asymptomatic for ovarian and/or endometrial cancer. In some embodiments, subjects are asymptomatic for endometrial cancer but do exhibit complex atypical hyperplasia (CAH). This is a pre-cancerous state (e.g., equivalent to stage 0 endometrial cancer) that is associated with an approximately 40% increased risk of a subject developing endometrial cancer. See e.g., Suh-Burgmann et al. et al. 2009 Obstetrics and Gynecology 114(3), 523-529. In some embodiments, the subject is symptomatic for ovarian and/or endometrial cancer. In some embodiments, a subject is from a population with an increased risk for ovarian and/or endometrial cancer. In some embodiments, the increased risk is that the subject has Lynch syndrome, the subject is obese, the subject has family history of ovarian and/or endometrial cancer, the subject has a BRCA mutation, and/or the subject is over a predetermined age—e.g., where the predetermined age is at least 40, at least 45, at least 50, at least 55, at least 60, at least 65, or at least 70 years of age). In some embodiments, the subject is asymptomatic. In some embodiments, the subject is experiencing pelvic pain, abnormal bleeding, or infertility.

In some embodiments, a subject is concurrently evaluated for a stage of an additional cancer condition distinct from ovarian and endometrial cancer. In some embodiments, another cancer condition is selected from the group consisting of lung cancer, prostate cancer, colorectal cancer, renal cancer, cancer of the esophagus, cervical cancer, bladder cancer, gastric cancer, nasopharyngeal cancer, or a combination thereof.

Referring to block 1404, the evaluation method proceeds by obtaining a fluid sample, e.g., a blood plasma or uterine lavage fluid, from the subject. In some embodiments, a uterine lavage fluid is collected from the subject via hysteroscopy combined with curettage. In some embodiments, uterine lavage fluid is collected from the subject via uterine washings.

In some embodiments, a second biological fluid is collected from the subject. In some embodiments, the second biological fluid is a lavage fluid. In some embodiments, the lavage fluid sample is a bronchoalveolar lavage fluid sample, a gastric lavage fluid sample, a ductal lavage fluid sample, a nasal irrigation sample, a peritoneal lavage fluid sample, a peritoneal lavage fluid sample, an arthroscopic lavage fluid sample, or ear lavage fluid sample. In some embodiments, the second biological fluid is blood or a fraction thereof, such as a blood plasma fraction.

In some embodiments, a body cavity from which the lavage fluid sample is collected determines which type(s) of cancer said lavage fluid sample is assayed for (e.g., bladder cancer, oral cancer, lung cancer, gastrointestinal cancer, endometrial, and/or ovarian). In some such embodiments, the method further evaluates the subject for a stage of bladder cancer, a stage of oral cancer, a stage of lung cancer, a stage of gastrointestinal cancer, a stage of endometrial cancer, and/or a stage of ovarian cancer, respectively.

Referring to block 1406, the evaluation method continues by determining, for each autoantibody species in a first set of autoantibody species, a corresponding abundance value for the respective autoantibody species in the biological fluid sample. The method thereby includes obtaining an autoantibody abundance dataset for the subject.

Table 2 lists features found to be informative for distinguishing between (i) the presence of either an endometrial cancer or an ovarian cancer and (ii) no endometrial cancer or ovarian cancer. Each feature represents a ratio of (i) the log of the abundance of the first listed gene, to (ii) the log of the abundance of the second listed gene. For instance, feature FGF7_DAD1 refers to a comparison (e.g., a ratio) of (i) the log abundance of autoantibodies that bind to the human FGF7 protein in a biological fluid sample, to (ii) the log abundance of autoantibodies that bind to the human DAD1 protein in the biological fluid sample. Accordingly, in some embodiments, the first set of autoantibody species includes an autoantibody species that binds to the human FGF7 protein. Similarly, in some embodiments, the first set of autoantibody species includes an autoantibody species that binds to the human DAD1 protein. Likewise, in some embodiments, the first set of autoantibody species includes an autoantibody species that binds to the human FGF7 protein and an autoantibody species that binds to the human DAD1 protein.

In some embodiments, the first set of autoantibody species includes at least 3 autoantibody species. In some embodiments, each respective autoantibody species of the at least 3 autoantibody species specifically binds to a different molecular target selected from those listed in Table 2. In some embodiments, the first set of autoantibody species includes at least 5 autoantibody species. In some embodiments, each respective autoantibody species of the at least 5 autoantibody species specifically binds to a different molecular target selected from those listed in Table 2. In some embodiments, the first set of autoantibody species includes at least 10 autoantibody species. In some embodiments, each respective autoantibody species of the at least 10 autoantibody species specifically binds to a different molecular target selected from those listed in Table 2. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 2.

TABLE 2
Example features found to be informative for distinguishing between
(i) the presence of either an endometrial cancer or an ovarian cancer and
(ii) no endometrial cancer or ovarian cancer. Each feature represents
a ratio of (i) the log of the abundance of the first listed gene,
to (ii) the log of the abundance of the second listed gene.
Example Features

FGF7_{——}DAD1
DAD1_{——}RIMS4
FGF7_{——}PLA2G16
LOC283951_{——}ACER1
DAD1_{——}ACTC1
LOC283951_{——}PFN3
HIGD2B_{——}OR5M3
LOC283951_{——}PAQR6
ROR1_{——}PAQR6
CLDN20_{——}dJ402G11.C22.5
EFNA4_{——}PAQR6
CLDN20_{——}TBX10
DCLRE1B_{——}ATP13A1
dJ402G11.C22.5_{——}PFN3
RERGL_{——}IFIT1B
DCLRE1B_{——}EBP
RERGL_{——}POLQ
CKMT2_{——}ACTC1
TPRG1_{——}LOC283951
MAP3K13_{——}HIGD2B
FGF7_{——}CKMT2
BARX1_{——}BTN2A3P
ST8SIA1_{——}PAQR6
GRPR_{——}SLC25A37
OR8H1_{——}NKX2-6
TTC39A_{——}TXN2
DCLRE1B_{——}BC013178_frag
ATAD3A_{——}TPRG1
TPRG1_{——}BTN2A3P
CYTH3_{——}TPRG1
ACTC1_{——}EMC4
TRIM40_{——}OCLN
TTC39A_{——}ATAD3A
DDRGK1_{——}CLDN20
OR8H1_{——}ANO2
TPRG1_{——}PHOSPHO2
CLDN20_{——}POT1
BRMS1L_{——}RIMS4
OCLN_{——}SUPT5H
TTC39A_{——}LOC283951
RERGL_{——}UST
TPRG1_{——}ECI1
ATP2B4_{——}GOLGA7B
TSPAN17_{——}LOC283951
SYT1_{——}CLDN20
TRIM40_{——}EID1
CLDN20_{——}NAALADL1
DBT_{——}LOC552889_frag
EFNA4_{——}MYO19
PPM1D_{——}CLDN20
BCKDHB_{——}CLDN20
CLDN20_{——}CTSL3P
SLC25A37_{——}PTGFRN
BRMS1L_NT5E
RPL38_{——}STX3
SPINK13_{——}SPINK8
METTL2B_{——}CLDN20
SRCRM_{——}CLDN20
ACTC1_{——}INO80C
LOC105372481_frag_{——}OCLN
ATP13A1_{——}RNPEPL1
LILRA2_{——}CLDN20
NDUFB10_{——}CLDN20
ST3GAL5_{——}PAQR6
GATAD2B_{——}DGKD
PLAC8_U2AF1
ACVR1B_{——}PAQR6
USP12_{——}PAQR6
GBA2_{——}NKX2-6
KJ902277_frag_{——}CLDN20
RNH1_{——}LIMS2_frag
MAP3K13_{——}SPATA5L1
CMPK1_{——}NUP58
RANBP17_{——}FKRP
JMJD7_{——}SPINK13
B3GALT5-AS1_{——}FBXL17
C1orf21_{——}EID1
PDP2_{——}CLDN20
NUPL2_{——}GCDH
SLC35F6_frag_{——}CLDN20
AIFM2_{——}ERBB4
OR8D4_{——}CLDN20
C1R_{——}CEP41
ELOF1_TMEM91
CIDEB_{——}OCLN
TUSC3_{——}ATP2B4
SLAMF8_{——}ACER1
C1orf61_{——}LINC00588
IFIT1B_{——}CPD
BPY2_{——}LOC283951
PLEKHG5_{——}EID1
RPL38_{——}C1R
NEURL3_frag_{——}OCLN
ATP6V1H_{——}CLDN20
RARRES3_{——}CLDN20
SLC25A48_{——}ERBB4
ZC3HAV1L_{——}CLDN20
HIPK3_{——}CLDN20
UBE2G2_{——}TAS2R50
ZNF509_{——}POLQ
KIAA1456_{——}EID1
ANKH_{——}CLDN20
CTXN1_{——}CLDN20
BRD3_{——}SPINK13
PORCN_{——}CCNA1
METTL2A_{——}KCNAB2
FEN1_{——}OCLN
USP44_{——}METTL8
SPESP1_{——}RANBP17
DNAJC16_{——}ZC2HC1B
SRPRB_{——}CLDN20
FBXO22_{——}LINC00588
HTR3A_{——}CLDN20
SLC2A12_{——}FOXF1
PRKACG_{——}FAM231D
TRIM31_frag_{——}ATP2B4
PBRM1_frag_{——}LOC552889_frag
ATP6V1B2_{——}SETD9

Table 3 lists features found to be informative for distinguishing between (i) the presence of endometrial cancer and (ii) all other gynecological conditions in the training set. Each feature represents a ratio of (i) the log of the abundance of the first listed gene, to (ii) the log of the abundance of the second listed gene. For instance, feature ZNF185_DGKH refers to a comparison (e.g., a ratio) of (i) the log abundance of autoantibodies that bind to the human ZNF185 protein in a biological fluid sample, to (ii) the log abundance of autoantibodies that bind to the human DGKH protein in the biological fluid sample. Accordingly, in some embodiments, the first set of autoantibody species includes an autoantibody species that binds to the human ZNF185 protein. Similarly, in some embodiments, the first set of autoantibody species includes an autoantibody species that binds to the human DGKH protein. Likewise, in some embodiments, the first set of autoantibody species includes an autoantibody species that binds to the human ZNF185 protein and an autoantibody species that binds to the human DGKH protein.

In some embodiments, the first set of autoantibody species includes at least 3 autoantibody species. In some embodiments, each respective autoantibody species of the at least 3 autoantibody species specifically binds to a different molecular target selected from those listed in Table 3. In some embodiments, the first set of autoantibody species includes at least 5 autoantibody species. In some embodiments, each respective autoantibody species of the at least 5 autoantibody species specifically binds to a different molecular target selected from those listed in Table 3. In some embodiments, the first set of autoantibody species includes at least 10 autoantibody species. In some embodiments, each respective autoantibody species of the at least 10 autoantibody species specifically binds to a different molecular target selected from those listed in Table 3. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 3.

TABLE 3
Example features found to be informative for distinguishing
between (i) the presence of endometrial cancer and (ii) all other
gynecological conditions in the training set. Each feature represents
a ratio of (i) the log of the abundance of autoantibody species that
bind to the first listed gene, to (ii) the log of the abundance of
autoantibody species that bind to the second listed gene.
Example Features

ZNF185_{——}DGKH
DGKH_{——}LOC283951
SURF1_{——}DAD1
GBGT1_{——}Six3
GBGT1_{——}DGKH
GJA9_{——}GSC
KJ901803_{——}RREB1
GJA9_{——}Six3
SLC7A3_{——}GSC
USMG5_{——}RREB1
GTF2A1L_{——}LOC283951
SEMA6A_{——}Six3
GJA9_{——}RREB1
MAST1_{——}DGKH
OVCH1_{——}RREB1
OR2T10_{——}RREB1
OCLN_{——}CCDC6
GJA9_{——}GBX2
Six3_{——}LOC283951
HTR3A_{——}GSC
TMUB1_{——}Six3
KJ901803_{——}Six3
LOC552889_frag_{——}Six3
Six3_{——}ST8SIA1
RNPEPL1_{——}Six3
RABGAP1L_{——}DGKH
OCLN_{——}DGKH
SEMA6A_{——}DGKH
TBX10_{——}RREB1
BACE1_{——}DGKH
F2R_{——}RREB1
SGPP1_{——}DGKH
DDRGK1_{——}DGKH
DMPK_{——}DGKH
OR2T10_{——}TARBP1
LOC283951_{——}TARBP1
NDUFS2_{——}Six3
C1orf53_{——}Six3
OR2T10_{——}RHNO1
TSPAN17_{——}LOC283951
EFNA4_{——}MYO19
LOC283951_{——}PFN3
KJ901803_{——}GTF2A1L
RBMY2FP_frag_{——}RREB1
GRIN3A_{——}ZNF646
ATP13A1_{——}GNG7
STK10_frag_{——}Six3
GABRB3_{——}RREB1
POLDIP2_{——}GTF2A1L
CTSD_{——}ZNF646
SLC7A3_{——}DGKH
LRRTM2_{——}RREB1
AGBL4_{——}RREB1
TBX10_{——}NOL9
KNCN_frag_{——}DGKH
RNPEPL1_{——}ZNF646
PALM_{——}ERBB4
LRRTM3_{——}ZNF646
KJ903857_frag_{——}DGKH
WARS2_{——}Six3
GDF3_{——}RREB1
HDDC3_{——}GJA9
LOC283951_{——}CUL5
DDRGK1_{——}RREB1
GJA9_{——}KRT81
C6orf1_{——}Six3
XM_004049765.1_frag_{——}RREB1
GNB1L_{——}LTBR
OVCH1_{——}NKX1-2
MRPS2_{——}RREB1
CCDC184_{——}LOC283951
KIAA2022_{——}LOC283951
ATP13A1_{——}ARHGEF5
OR11G2_{——}OR4N4
RND2_{——}ERBB4
KLHL29_{——}ZNF646
ATP13A1_{——}KJ900931
PXYLP1_{——}ERBB4
SSX4_{——}ERBB4
LHX3_{——}OR5211
VTI1B_{——}TMEM8B
FBXO22_{——}RREB1
COX5BP4_frag_{——}ENPP1
FGFR4_{——}RREB1
HMSD_{——}Six3
ST3GAL5_{——}PAQR6
LINC00471_{——}LINC00588
SPESP1_{——}ICAp69
RABGAP1L_{——}JHU19590
LOC283951_{——}TRPC1
HIPK4_{——}ZNF646
CERS1_{——}Eomes
LINC00610_{——}RREB1
LY6G6F_{——}RREB1
IDH3B_{——}LINC00588
RMI1_{——}Six3
C8orf45_{——}RREB1
ACP1_{——}FDCSP
NMUR1_{——}ENOSF1
AC074325.7_frag_{——}RREB1
YPEL3_{——}ERBB4
IRX6_{——}SSX4
PSMD12_{——}KRT81
C2orf57_{——}MDFIC
FGGY_{——}F8
CCDC184_{——}SEMA7A

Table 4 lists features found to be informative for distinguishing between (i) the presence of endometrial cancer and (ii) a benign gynecological condition. Each feature represents a ratio of (i) the log of the abundance of the first listed gene, to (ii) the log of the abundance of the second listed gene. For instance, feature SURF1_DAD1 refers to a comparison (e.g., a ratio) of (i) the log abundance of autoantibodies that bind to the human SURF1 protein in a biological fluid sample, to (ii) the log abundance of autoantibodies that bind to the human DAD1 protein in the biological fluid sample. Accordingly, in some embodiments, the first set of autoantibody species includes an autoantibody species that binds to the human SURF1 protein. Similarly, in some embodiments, the first set of autoantibody species includes an autoantibody species that binds to the human DAD1 protein. Likewise, in some embodiments, the first set of autoantibody species includes an autoantibody species that binds to the human SURF1 protein and an autoantibody species that binds to the human DAD1 protein.

In some embodiments, the first set of autoantibody species includes at least 3 autoantibody species. In some embodiments, each respective autoantibody species of the at least 3 autoantibody species specifically binds to a different molecular target selected from those listed in Table 4. In some embodiments, the first set of autoantibody species includes at least 5 autoantibody species. In some embodiments, each respective autoantibody species of the at least 5 autoantibody species specifically binds to a different molecular target selected from those listed in Table 4. In some embodiments, the first set of autoantibody species includes at least 10 autoantibody species. In some embodiments, each respective autoantibody species of the at least 10 autoantibody species specifically binds to a different molecular target selected from those listed in Table 4. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 4.

TABLE 4
Example features found to be informative for distinguishing
between (i) the presence of endometrial cancer and (ii) a benign
gynecological condition. Each feature represents a ratio of (i)
the log of the abundance of autoantibody species that bind to
the first listed gene, to (ii) the log of the abundance of
autoantibody species that bind to the second listed gene.
Example Features

SURF1_{——}DAD1
ZNF185_{——}DGKH
MAST1_{——}DGKH
DGKH_{——}LOC283951
GTF2A1L_{——}LOC283951
OCLN_{——}CCDC6
LOC283951_{——}PAQR6
RERGL_{——}MYO19
LOC283951_{——}PFN3
LOC283951_{——}ACER1
OCLN_{——}DGKH
DCLRE1B_{——}ATP13A1
LOC283951_{——}POLQ
RNF215_{——}Six3
GBGT1_{——}DGKH
KJ901803_{——}SLC25A37
ST8SIA1_{——}PAQR6
DAD1_{——}TSPO
C1orf53_{——}ANKRD20A5P
KJ901803_{——}RREB1
EFNA4_{——}PAQR6
LOC283951_{——}PLEKHF1
LOC283951_{——}IFIT1B
KJ901803_{——}METTL8
KJ901803_{——}PAQR6
EFNA4_{——}MYO19
RERGL_{——}POLQ
OCLN_{——}GTF2A1L
ST8SIA1_{——}TARBP1
DCLRE1B_{——}SGOL1
SEMA6A_{——}DGKH
KJ901803_{——}GTF2A1L
RERGL_{——}IFIT1B
TSPAN17_{——}LOC283951
RABGAP1L_{——}DGKH
LOC283951_{——}TARBP1
ECI1_{——}ZWILCH
LOC283951_{——}ZNF726
TAS2R40_{——}ERBB4
GATAD2B_{——}DGKH
CLCNKA_{——}Six3
TMIGD3_{——}POLQ
USMG5_{——}RREB1
GATS_{——}MYO19
KMO_{——}TCF7
ATP2B4_{——}GOLGA7B
LRRTM3_{——}ZNF646
KNCN_frag_{——}DGKH
KJ903857_frag_{——}GTF2A1L
FAM71C_{——}BANF2
C1R_{——}PAQR6
KJ903857_frag_{——}DGKH
CTSD_{——}ZNF646
DDRGK1_{——}CLDN20
RERGL_{——}IGFBP5
PRRX2_{——}ECI1
KNCN_frag_{——}GTF2A1L
DBT_{——}LOC552889_frag
HGSNAT_{——}Six3
ATP13A1_{——}ARHGEF5
OR8H1_{——}ANO2
ST3GAL5_{——}PAQR6
POLDIP2_{——}GTF2A1L
CYTH3_{——}TPRG1
OR11G2_{——}OR4N4
OCLN_{——}Pou3f1
USP12_{——}PAQR6
LPCAT3_{——}CLDN20
TMUB1_{——}TMEM8B
SSX4_{——}ERBB4
LINC00471_{——}LINC00588
BRMS1L_{——}NT5E
JMJD7_{——}SPINK13
EML1_{——}GTF2A1L
LOC105372481_frag_{——}OCLN
PALM_{——}ERBB4
LHX3_{——}OR52I1
BRMS1L_{——}RIMS4
SLC25A37_{——}PTGFRN
USP12_{——}ERBB4
TBX10_{——}B3GALT5-AS1
ACVR1B_{——}PAQR6
OVCH1_{——}HOXD11
OVCH1_{——}NKX1-2
LOC283951_{——}KCNH7
NKAIN4_{——}LOC283951
ARR3_{——}TMEM8B
B3GNT6_{——}MAB21L3
ABCA9_{——}LOC283951
SLC25A48_{——}ERBB4
KDELR1_{——}CLCNKB
FBXO22_{——}RREB1
CAPN11_{——}LINC00588
ST3GAL5_{——}IFIT1B
XM_004049765.1_frag_{——}RREB1
NDUFS2_{——}ENOSF1
ZNF35_{——}ECI1
VTI1B_{——}TMEM8B
USP12_{——}GATM
WDR60_{——}UST
RND2_{——}ERBB4
ANKH_{——}CLDN20
ABHD17A_{——}ADCYAP1
KLHL29_{——}ZNF646
RBMY2FP_frag_{——}LINC00588
ADAMTS12_{——}TMEM8B
CDK3_{——}TMEM8B
LYZL2_{——}PAQR6
USP44_METTL8
EFNA4_{——}RLTPR
NDUFV1_{——}BRMS1L
TSPAN9_frag_{——}Six3
BGN_{——}PAX3
YPEL3_{——}ERBB4
GBA2_{——}NKX2-6
CMAS_{——}NUP58
PALM_{——}PAX3
SCN5A_{——}LOC283951
NMUR1_{——}ENOSF1
EPX_{——}LOC283951
C2orf57_{——}MDFIC
PBRM1_frag_{——}LOC552889_frag

Table 5 lists features found to be informative for distinguishing between (i) the presence of ovarian cancer and (ii) all other gynecological conditions in the training set. Each feature represents a ratio of (i) the log of the abundance of the first listed gene, to (ii) the log of the abundance of the second listed gene. For instance, feature SMAD1_MTHFR refers to a comparison (e.g., a ratio) of (i) the log abundance of autoantibodies that bind to the human SMAD1 protein in a biological fluid sample, to (ii) the log abundance of autoantibodies that bind to the human MTHFR protein in the biological fluid sample. Accordingly, in some embodiments, the first set of autoantibody species includes an autoantibody species that binds to the human SMAD1 protein. Similarly, in some embodiments, the first set of autoantibody species includes an autoantibody species that binds to the human MTHFR protein. Likewise, in some embodiments, the first set of autoantibody species includes an autoantibody species that binds to the human SMAD1 protein and an autoantibody species that binds to the human MTHFR protein.

In some embodiments, the first set of autoantibody species includes at least 3 autoantibody species. In some embodiments, each respective autoantibody species of the at least 3 autoantibody species specifically binds to a different molecular target selected from those listed in Table 5. In some embodiments, the first set of autoantibody species includes at least 5 autoantibody species. In some embodiments, each respective autoantibody species of the at least 5 autoantibody species specifically binds to a different molecular target selected from those listed in Table 5. In some embodiments, the first set of autoantibody species includes at least 10 autoantibody species. In some embodiments, each respective autoantibody species of the at least 10 autoantibody species specifically binds to a different molecular target selected from those listed in Table 5. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 5.

TABLE 5
Example features found to be informative for distinguishing
between (i) the presence of ovarian cancer and (ii) all other
gynecological conditions in the training set. Each feature
represents a ratio of (i) the log of the abundance of autoantibody
species that bind to the first listed gene, to (ii) the log of the
abundance of autoantibody species that bind to the second listed gene.
Example Features

CCAR2_{——}KIAA0368_frag
SMAD1_{——}MTHFR
GNG7_{——}FOXI2
TYMSOS_{——}STAG3_frag
PKLR_{——}MTHFR
CCAR2_{——}MTHFR
KIAA2022_{——}ZMIZ1
TYMSOS_{——}TET1
DCLK1_{——}MTHFR
ZG16B_{——}MTHFR
F8_{——}RGL1
AMPH_{——}MTHFR
PANK1_frag_{——}CLTCL1
TYMSOS_{——}ARL4C
PKLR_{——}PAWR
TYMSOS_{——}HIST1H2AJ
CBFA2T2_{——}FAM9B
SUN1_{——}KIAA2022
RPS4Y1_{——}CLTCL1
ALDH4A1_{——}KIAA0368_frag
RNF151_{——}MTHFR
ZDHHC6_{——}MTHFR
ECH1_{——}PLK1
TYMSOS_{——}LAMTOR1
PUS3_{——}MTHFR
RPS4Y1_{——}BSPH1
TMEM101_{——}MTHFR
CCAR2_{——}ZNF606
GNG7_{——}FBXL19-AS1
FMR1_{——}HACE1
ZFP2_{——}CCAR2
CLCN1_{——}MTHFR
CCAR2_{——}SPATA8
ZNF354C_{——}CCAR2
ZIC3_{——}MTHFR
ZG16B_{——}PLK1
DCLK1_{——}ZNF816
ZNF613_{——}CCAR2
RPS4Y1_{——}RMI2
ZNF25_{——}CCAR2
TYMSOS_{——}KLF9
TYMSOS_{——}KIFC1
ZNF468_{——}CCAR2
WFS1_{——}HIST1H2AJ
THUMPD1_{——}FAM9B
DCLK1_{——}EDC4
CCAR2_{——}Q91wf9
ZC3HC1_{——}STAG3_frag
KIAA2022_{——}PUS3
CCAR2_{——}NSUN4
PRR14_{——}MTHFR
ZNF19_{——}ARL4C
SGPP1_{——}MTHFR
HACE1_{——}BECN1
ABLIM1_{——}KIAA0368_frag
ABHD5_{——}HACE1
GRB10_{——}WFS1
CCAR2_{——}TIMM50
DAP_{——}PUS3
GNG7_{——}ARL4C
OSBPL5_{——}ARL4C
MRPS21_{——}NANOGP8
OSBPL5_{——}KIFC1
ZNF630_{——}CCAR2
GNG7_{——}KLF9
HACE1_{——}ZNF184
RPH3A_{——}ISG20L2
ALDH4A1_{——}ZNF606
CCAR2_{——}ZNF578
CCAR2_{——}MRPL53
CCAR2_{——}ZNF33A
WFS1_{——}PIK3IP1
HACE1_{——}PLEKHA6
GPR1_{——}RGL1
CCAR2_{——}LINC00242
CCAR2_{——}SCAND1
SPAG11A_{——}CCAR2
NFAT5_{——}ZNF816
CCAR2_{——}ZNF793
CCAR2_{——}RACGAP1
CCAR2_{——}ZNF823
MTHFR_{——}CEMIP
DTX1_{——}CCAR2
WFS1_{——}InfluenzaAM2
CCAR2_{——}YF006
RNASE2_{——}MTHFR
SULF1_{——}CCAR2
CCAR2_{——}ZNF197
STAG3_frag_{——}CCAR2
CCAR2_{——}ZNF816
KLF10_{——}KIAA2022
BUD13_{——}MTHFR
NCBP3_{——}HIST1H2AJ
ZNF592_{——}HACE1
NFAT5_{——}HIST1H2AJ
CCAR2_{——}ZNF583
CCAR2_{——}ZNF229
HACE1_{——}KIAA2022
DCLK1_{——}LCE3D
ZNF184_{——}PUS3
FLYWCH2_{——}KIAA2022
FAM46D_{——}FGF20
CCAR2_{——}SENP7
KCNK10_{——}CCDC142
ITK_{——}MTHFR
WFS1_{——}PLA2G5
LRRC3B_{——}PLEKHA6
WFS1_{——}ARL4C
CCAR2_{——}GCM1
RIMS3_{——}HACE1
HIGD1A_{——}TUBB8
PUS3_{——}PLEKHA6
LRRC3B_{——}STAG3_frag
CCAR2_{——}ZNF562
HMBS_{——}PUS3
CCAR2_{——}HAS1
CCAR2_{——}ZNF615
FAM124A_{——}PUS3
HACE1_{——}CFAP74
WFS1_{——}KJ903245
CSF2_{——}CCAR2
CCAR2_{——}ARL4C
ZNF182_{——}CCAR2
TNFSF10_{——}CCAR2
HIST1H2AJ_{——}CCAR2
CCAR2_{——}ZNF112
CCAR2_{——}ZNF814
SLC2A12_{——}CCAR2
ZNF34_{——}CCAR2
PRIM2_{——}CLTCL1
FANCC_{——}CCAR2
ABHD10_{——}CCAR2
ZNF702P_{——}CCAR2
NFAT5_{——}ARL4C
LRRC3B_{——}FOXI2
DUSP13_{——}CCAR2
NFAT5_{——}KJ903245
BATF_{——}CCAR2
HACE1_{——}S100A3
WFS1_{——}LOC338797
MT1DP_{——}CCAR2
CCAR2_{——}ZNF638
STAG3_frag_{——}PUS3
LRRC3B_{——}PLA2G5
NFAT5_{——}KJ902965_frag
CCAR2_{——}ZDHHC1
CCAR2_{——}INSM1
CCAR2_{——}DEPDC5
SOSTDC1_{——}CCAR2
LRRC3B_{——}SENP7
CCDC15_frag_{——}CCAR2
LRRC3B_{——}ARL4C
LRRC3B_{——}LAMTOR1
LRRC3B_{——}PCDH11Y

Table 6 lists features found to be informative for distinguishing between (i) the presence of ovarian cancer and (ii) a benign gynecological condition. Each feature represents a ratio of (i) the log of the abundance of the first listed gene, to (ii) the log of the abundance of the second listed gene. For instance, feature ZG16B_MTHFR refers to a comparison (e.g., a ratio) of (i) the log abundance of autoantibodies that bind to the human ZG16B protein in a biological fluid sample, to (ii) the log abundance of autoantibodies that bind to the human MTHFR protein in the biological fluid sample. Accordingly, in some embodiments, the first set of autoantibody species includes an autoantibody species that binds to the human ZG16B protein. Similarly, in some embodiments, the first set of autoantibody species includes an autoantibody species that binds to the human MTHFR protein. Likewise, in some embodiments, the first set of autoantibody species includes an autoantibody species that binds to the human ZG16B protein and an autoantibody species that binds to the human MTHFR protein.

In some embodiments, the first set of autoantibody species includes at least 3 autoantibody species. In some embodiments, each respective autoantibody species of the at least 3 autoantibody species specifically binds to a different molecular target selected from those listed in Table 6. In some embodiments, the first set of autoantibody species includes at least 5 autoantibody species. In some embodiments, each respective autoantibody species of the at least 5 autoantibody species specifically binds to a different molecular target selected from those listed in Table 6. In some embodiments, the first set of autoantibody species includes at least 10 autoantibody species. In some embodiments, each respective autoantibody species of the at least 10 autoantibody species specifically binds to a different molecular target selected from those listed in Table 6. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 6.

TABLE 6
Example features found to be informative for distinguishing
between (i) the presence of ovarian cancer and (ii) a benign
gynecological condition. Each feature represents a ratio of
(i) the log of the abundance of autoantibody species that bind
to the first listed gene, to (ii) the log of the abundance of
autoantibody species that bind to the second listed gene.
Example Features

ZG16B_{——}MTHFR
CCAR2_{——}KIAA0368_frag
CCAR2_{——}MTHFR
STAG3_frag_{——}NKX2-6
PKLR_{——}MTHFR
AMPH_{——}MTHFR
TYMSOS_{——}STAG3_frag
CMKLR1_{——}STAG3_frag
DCLK1_{——}MTHFR
ABLIM1_{——}MTHFR
OSBPL5_{——}ARPP19
ACCS_{——}MTHFR
SMAD1_{——}MTHFR
GADD45G_{——}TFG
TMEM101_{——}ICAM3
TERF1_{——}CCAR2
LRRC3B_{——}MTHFR
SNX32_{——}ICAM3
MTHFR_{——}ZMIZ1
CCAR2_{——}ZNF606
ZNF354C_{——}CCAR2
PPP1R13B_{——}MTHFR
PUS3_{——}MTHFR
RNF151_{——}MTHFR
DCPS_{——}MTHFR
GADD45G_{——}RPH3A
NDUFB6_{——}ICAM3
TMEM101_{——}MTHFR
PANK1_frag_{——}CLTCL1
ZDHHC6_{——}MTHFR
TYMSOS_{——}ARL4C
F8_{——}MTHFR
COX15_{——}MTHFR
PRAMEF5_{——}CD180
TGIF2_{——}MTHFR
SNX32_{——}TUBAL3
ECH1_{——}MTHFR
MTHFR_{——}DCAF8L2
MTHFR_{——}PRR20A
CNTNAP1_{——}MTHFR
MAPK8IP2_{——}MTHFR
PKLR_{——}FKRP
DNAJC30_{——}MTHFR
RNF151_{——}ERBB2
ZC3HC1_{——}STAG3_frag
ZIC3_{——}MTHFR
MTHFR_{——}NCOR1
PHACTR3_{——}CCAR2
APOO_{——}MTHFR
MTHFR_{——}DHX33
PKLR_{——}PAWR
STAG3_frag_{——}CCAR2
C1orf64_{——}MTHFR
ARL13B_{——}MTHFR
AQP7_{——}MTHFR
DTX1_{——}CCAR2
NTSR1_{——}MTHFR
SEC61B_{——}MTHFR
PLPPR2_{——}MTHFR
MYOZ1_{——}MTHFR
CCAR2_{——}RACGAP1
MCF2_{——}MTHFR
CCAR2_{——}MAP3K13
LAMTOR1_{——}NKX2-6
TMEM160_{——}MTHFR
FITM1_{——}MTHFR
CCAR2_{——}SCAND1
WFDC3_frag_{——}PKLR
ZNF468_{——}CCAR2
ARV1_{——}MTHFR
CLCN1_{——}MTHFR
TACO1_{——}MTHFR
MTHFR_{——}KCNH7
SCPEP1_{——}MTHFR
NIPAL4_{——}MTHFR
DERL3_{——}MTHFR
KCNQ3_{——}MTHFR
MRPS14_{——}MTHFR
CCAR2_{——}ZNF578
ANKRD29_{——}MTHFR
MTHFR_{——}SLC2A1
HSPB3_{——}MTHFR
ARL4C_{——}NKX2-6
LINC01558_{——}STAG3_frag
RAPH1_{——}MTHFR
CASR_{——}MTHFR
GADD45G_{——}F2
PRR14_{——}MTHFR
BMP15_{——}MTHFR
DEFB1_{——}MTHFR
RPL15_{——}MTHFR
MTHFR_{——}TSSK6
SPPL2B_{——}MTHFR
CCAR2_{——}ZNF33A
TAS2R39_{——}MTHFR
CBFA2T2_{——}FAM9B
OR51D1_{——}MTHFR
MTHFR_{——}C16orf46
SGPP1_{——}MTHFR
ERAS_{——}MTHFR
MTHFR_{——}KCNQ1DN
FAM19A4_{——}MTHFR
LRRC3B_{——}STAG3_frag
SPAG11A_{——}CCAR2
KJ901253_frag_{——}MTHFR
MTHFR_{——}PLSCR5
FAM19A3_{——}MTHFR
GADD45G_{——}MPC2
HCN3_{——}MTHFR
ACER1_{——}FBXL17
CCAR2_{——}ZNF816
MTHFR_{——}GPR139
GPR83_{——}MTHFR
OSBPL5_{——}KIFC1
SULT1C3_{——}MTHFR
KJ903261_{——}MTHFR
RASGRP4_{——}MTHFR
DSC1_{——}MTHFR
CYBA_{——}MTHFR
BNC2_{——}MTHFR
LRRC3B_{——}ICAM3
CMTM4_{——}MTHFR
EU831996_{——}MTHFR
CD163_{——}MTHFR
PSG8_{——}MTHFR
LINC01104_frag_{——}MTHFR
ILDR1_{——}MTHFR
PTGIR_{——}MTHFR
SNX32_{——}PRICKLE4
MTHFR_{——}PITPNM3
MTHFR_{——}MTHFSD
HTR4_{——}MTHFR
SLC2A12_{——}CCAR2
GADD45G_{——}HINT2
LN607916.1_frag_{——}LRRC3B
RNASE2_{——}MTHFR
SLC32A1_{——}MTHFR
WFS1_{——}HIST1H2AJ
CCAR2_{——}ZNF229
ITK_{——}MTHFR
WFS1_{——}ARL4C
YIF1A_{——}MTHFR
ZNF285_{——}MTHFR
MLPH_{——}MTHFR
C17orf50_{——}MTHFR
GABRR1_{——}MTHFR
NFAT5_{——}HIST1H2AJ
FANCC_{——}CCAR2
ERVK13-1_{——}MTHFR
ZNF182_{——}CCAR2
NFAT5_{——}ARL4C
XM_004049765.1_frag_{——}MTHFR

Table 7 lists features found to be informative for distinguishing between (i) the presence of ovarian cancer and (ii) the presence of endometrial cancer. Each feature represents a ratio of (i) the log of the abundance of the first listed gene, to (ii) the log of the abundance of the second listed gene. For instance, feature TYMSOS_TET1 refers to a comparison (e.g., a ratio) of (i) the log abundance of autoantibodies that bind to the human TYMSOS protein in a biological fluid sample, to (ii) the log abundance of autoantibodies that bind to the human TET1 protein in the biological fluid sample. Accordingly, in some embodiments, the first set of autoantibody species includes an autoantibody species that binds to the human TYMSOS protein. Similarly, in some embodiments, the first set of autoantibody species includes an autoantibody species that binds to the human TET1 protein. Likewise, in some embodiments, the first set of autoantibody species includes an autoantibody species that binds to the human TYMSOS protein and an autoantibody species that binds to the human TET1 protein.

In some embodiments, the first set of autoantibody species includes at least 3 autoantibody species. In some embodiments, each respective autoantibody species of the at least 3 autoantibody species specifically binds to a different molecular target selected from those listed in Table 7. In some embodiments, the first set of autoantibody species includes at least 5 autoantibody species. In some embodiments, each respective autoantibody species of the at least 5 autoantibody species specifically binds to a different molecular target selected from those listed in Table 7. In some embodiments, the first set of autoantibody species includes at least 10 autoantibody species. In some embodiments, each respective autoantibody species of the at least 10 autoantibody species specifically binds to a different molecular target selected from those listed in Table 7. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 7.

TABLE 7
Example features found to be informative for distinguishing between (i)
the presence of ovarian cancer and (ii) the presence of endometrial cancer.
Each feature represents a ratio of (i) the log of the abundance of
autoantibody species that bind to the first listed gene, to (ii) the log of the
abundance of autoantibody species that bind to the second listed gene.
Example Features

TYMSOS_TET1
TYMSOS_GBX2
GNG7_FOXI2
TYMSOS_H3F3C
RNF149_KIAA2022
TYMSOS_HIST1H2AJ
OR10A3_ADIG
RPS4Y1_BSPH1
ARF3_KIAA2022
LEF1_ZDHHC6
LEF1_RAC1
FAM46D_FGF20
C19orf53_FGF20
STARD3_DLX1
PKLR_PAWR
PANK1_frag_CLTCL1
LEF1_F8
VN1R5_GDF2
TYMSOS_BHLHE40
LEF1_RAB13
CAPRIN1_KIAA2022
ABHD5_HACE1
LEF1_MRAS
THUMPD1_FAM9B
SOHLH1_KIAA2022
ELF5_KIAA2022
MS4A12_ACP1
WFS1_HIST1H2AJ
LEF1_NF2_frag

Table 8 lists features found to be informative for distinguishing between (i) the presence of endometrial polyps and (ii) the absence of endometrial polyps. Each feature represents a ratio of (i) the log of the abundance of the first listed gene, to (ii) the log of the abundance of the second listed gene. For instance, feature SLFN5_CEP85 refers to a comparison (e.g., a ratio) of (i) the log abundance of autoantibodies that bind to the human SLFN5 protein in a biological fluid sample, to (ii) the log abundance of autoantibodies that bind to the human CEP85 protein in the biological fluid sample. Accordingly, in some embodiments, the first set of autoantibody species includes an autoantibody species that binds to the human SLFN5 protein. Similarly, in some embodiments, the first set of autoantibody species includes an autoantibody species that binds to the human CEP85 protein. Likewise, in some embodiments, the first set of autoantibody species includes an autoantibody species that binds to the human SLFN5 protein and an autoantibody species that binds to the human CEP85 protein.

In some embodiments, the first set of autoantibody species includes at least 3 autoantibody species. In some embodiments, each respective autoantibody species of the at least 3 autoantibody species specifically binds to a different molecular target selected from those listed in Table 8. In some embodiments, the first set of autoantibody species includes at least 5 autoantibody species. In some embodiments, each respective autoantibody species of the at least 5 autoantibody species specifically binds to a different molecular target selected from those listed in Table 8. In some embodiments, the first set of autoantibody species includes at least 10 autoantibody species. In some embodiments, each respective autoantibody species of the at least 10 autoantibody species specifically binds to a different molecular target selected from those listed in Table 8. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 8.

TABLE 8
Example features found to be informative for distinguishing between
(i) the presence of endometrial polyps and (ii) the absence of endometrial
polyps. Each feature represents a ratio of (i) the log of the abundance of
autoantibody species that bind to the first listed gene, to (ii) the log of the
abundance of autoantibody species that bind to the second listed gene.
Example Features

SLFN5_CEP85
GAS7_CEP85
MFI2_CEP85
CEP85_FAM9A
CEP85_PHYHIP
CEP85_EIF5B
MFI2_YBX2
CEP85_ZNF408
SLC27A2_CEP85
CEP85_ZAK
YBX2_HIF1A
CEP85_DVL3
GAS7_KIFAP3
ZNF408_KIFAP3
RRM2_HIF1A
SLFN5_YBX2
CEP85_HIF1A
MFI2_KIFAP3
KIFAP3_HIF1A
YBX2_CDV3
TGFB111_CEP85
SCMH1_KIFAP3
SLC27A2_LAMTOR1
FNDC7_SERPINC1
YBX2_SCMH1
SLFN5_CDK10
GIT2_CEP85
CEP85_ERO1B
TMEM51_CEP85
MFI2_LZTS3
HIF1A_CNOT3
CHRM5_SLC27A2
SLC27A2_FNDC7
CEP85_ANKRD54
EIF5B_KIFAP3
SLFN5_RRM2
PHC3_CEP85
SLFN5_KIFAP3
MFI2_RRM2
AP1M2_KIFAP3
SLFN5_FNDC7
CEP85_ZNF136
HAUS8_CEP85
CEP85_KIF1BP
SLC27A2_KIFAP3
CEP85_HOXB5
SLC27A2_ATG9A
CEP85_THOC7
KIFAP3_ZAK
SLC27A2_IP6K2
SLC27A2_PEX6
ZNF408_CNOT3
CXCR3_FNDC7
SLFN5_POLR2E
KIFAP3_DENND2D
LZTS3_HIF1A
SLFN5_CNOT3
CEP85_AKAP5
SLC27A2_POLR2E
MRPL41_CEP85
SLC27A2_KJ902417
SLC27A2_SAG

Table 9 lists features found to be informative for distinguishing between (i) the presence of adenomyosis and (ii) the absence of adenomyosis. Each feature represents a ratio of (i) the log of the abundance of the first listed gene, to (ii) the log of the abundance of the second listed gene. For instance, feature POLR1D_ATP2B4 refers to a comparison (e.g., a ratio) of (i) the log abundance of autoantibodies that bind to the human POLR1D protein in a biological fluid sample, to (ii) the log abundance of autoantibodies that bind to the human ATP2B4 protein in the biological fluid sample. Accordingly, in some embodiments, the first set of autoantibody species includes an autoantibody species that binds to the human POLR1D protein. Similarly, in some embodiments, the first set of autoantibody species includes an autoantibody species that binds to the human ATP2B4 protein. Likewise, in some embodiments, the first set of autoantibody species includes an autoantibody species that binds to the human POLR1D protein and an autoantibody species that binds to the human ATP2B4 protein.

In some embodiments, the first set of autoantibody species includes at least 3 autoantibody species. In some embodiments, each respective autoantibody species of the at least 3 autoantibody species specifically binds to a different molecular target selected from those listed in Table 9. In some embodiments, the first set of autoantibody species includes at least 5 autoantibody species. In some embodiments, each respective autoantibody species of the at least 5 autoantibody species specifically binds to a different molecular target selected from those listed in Table 9. In some embodiments, the first set of autoantibody species includes at least 10 autoantibody species. In some embodiments, each respective autoantibody species of the at least 10 autoantibody species specifically binds to a different molecular target selected from those listed in Table 9. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 9.

TABLE 9
Example features found to be informative for distinguishing between
(i) the presence of adenomyosis and (ii) the absence of adenomyosis.
Each feature represents a ratio of (i) the log of the abundance of
autoantibody species that bind to the first listed gene, to (ii) the log of
the abundance of autoantibody species that bind to the second listed gene.
Example Features

POLR1D_ATP2B4
POLR1D_KRT79
LY6E_PATE2
UROD_DMRTB1_frag
KRT79_HSP90B1
OTUD6B_ZKSCAN8
LINC01465_ZBTB8A
ZKSCAN8_TBX20
MGAT4D_frag_PAX2
KRT79_TBX20
ZKSCAN8_DNMT3L
KRT79_CCDC138
AK097058.1_frag_PAX2
DNMT3L_DMRTB1_frag
UROD_LINC01465
ZKSCAN8_C17orf82
ZKSCAN8_CCDC138
NUMA1_TBX20
NME2_DMRTB1_frag
ZKSCAN8_AHR
ZKSCAN8_SPANXD
ZKSCAN8_PIAS4
ZKSCAN8_NR1H2
ZKSCAN8_ZBTB8A
LINC01465_PRM3
KJ903532_TET3
CCDC138_NUMA1
AK097058.1_frag_NPIPL1
POLR1D_ZKSCAN8
LINC01465_TBX20
POLR1D_UMOD
DNMT3L_CKM
SLC1A3_THBS2
CGB_KRT79
ABHD13_PAX2
OTUD6B_NUMA1
KRT79_TELO2
LINC01465_DGKD
A0A096LNS0_CYB561D1
TBX20_STAT2
LINC01465_NR1H2
DMRT1_PIAS4
CCDC138_LINC01465
KRT79_CGB2
SLC1A3_UMOD
POLR1D_PSD3_frag
XKR8_frag_KJ903532
SPANXC_UMOD
ATP2B4_HSP90B1
CCDC138_ZNF574
SOX9_LINC01465
KJ903532_OIT3
BPIFB1_UMOD
LINC01465_PER2
AGA_UMOD
POLR1D_THBS2
LINC01465_UTP23
KRT79_SLC1A3
LINC01465_C17orf82
LINC01465_C1orf106
SLC1A3_ATP2B4
POLR1D_ABHD13
POLR1D_AK097058.1_frag
IL18_LINC01465
YU004_LINC01465
POLR1D_TM6SF1
MBTPS2_PAX2
KRT79_PMPCA
UROD_SLC16A2
LINC01465_TRIM26
AK097058.1_frag_VAV3
LINC01465_SPOP
TTC23L_UMOD
CCDC138_PSD3_frag
BC017762_frag_LINC01465
DIEXF_AK097058.1_frag
TONSL_LINC01465
DIEXF_UMOD
ZKSCAN8_SPOPL
PRUNE_LINC01465
PLSCR1_LINC01465
LINC01465_ASB16-AS1
DMRTB1_frag_HSP90B1
NPM2_LINC01465
YU004_LPAR2
ABHD12_MGAT4D_frag
ZKSCAN8_TRMT10B
POLR1D_STAT2
POLR1D_TOMM40
FAM162A_AGA
LINC01465_IGFBP6
CBX8_LINC01465
OTUD6B_LINC01465
LINC01465_PIGB
YWHAB_NCS1
C19orf25_LINC01465
POLR1D_STK31
TUBB3_ATP2B4
TM6SF1_OPN1MW
LINC01465_RERE
MGAT4D_frag_TELO2
UROD_SH3BGRL3
TNNT2_LINC01465
ABHD12_AK097058.1_frag
POLR1D_BC070352.1_frag
LINC01465_PIAS4
ABHD13_GRIK1
LINC01465_RNF14
FGF1_LINC01465
SLC35A3_UMOD
POLR1D_KCNS1
POLR1D_DSTYK

Table 10 lists features found to be informative for distinguishing between (i) the presence of endometrial or ovarian cancer and (ii) the absence of endometrial or ovarian cancer. Each feature represents an abundance of a single autoantibody species that binds to the protein listed in a biological fluid. For instance, CHRNA1_JHU04147.B2C18R66 refers to a log abundance of autoantibodies that bind to the human CHRNA1 protein in a biological fluid. Age refers to the age of the subject and BMI refers to the body mass index of the subject.

In some embodiments, the first set of autoantibody species includes at least 3 autoantibody species. In some embodiments, each respective autoantibody species of the at least 3 autoantibody species specifically binds to a different molecular target selected from those listed in Table 10. In some embodiments, the first set of autoantibody species includes at least 5 autoantibody species. In some embodiments, each respective autoantibody species of the at least 5 autoantibody species specifically binds to a different molecular target selected from those listed in Table 10. In some embodiments, the first set of autoantibody species includes at least 10 autoantibody species. In some embodiments, each respective autoantibody species of the at least 10 autoantibody species specifically binds to a different molecular target selected from those listed in Table 10. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 10.

TABLE 10
Example features found to be informative for distinguishing
between (i) the presence of endometrial or ovarian cancer
and (ii) the absence of endometrial or ovarian cancer.
Example Features

Age
BMI
CHRNA1_JHU04147.B2C18R66
CCDC47_JHU18441.B16C10R86
C11orf65_JHU04426.B2C16R70
GRID1_JHU06088.B6C18R2
ALDH2_JHU04131.B2C13R66
CPSF7_JHU04072.B2C14R66
PFN1_JHU14579.B9C26R50
ACVRL1_JHU04035.B2C4R62
SPRR2E_JHU17584.B14C32R58
TRPT1_JHU04502.B2C6R72
PLD3_JHU04101.B2C4R66
RPS26_JHU09191.B6C13R54
ASB4_JHU10082.B7C14R72
APOBEC3F_JHU14364.B9C19R44
CBARP_JHU04328.B2C9R70
CRYZL1_JHU02802.B1C27R44
EMC1_JHU14211.B12C20R44
AC013402.2_frag_JHU10746.B7C21R76
CANT1_JHU01276.B1C17R20
MECP2_JHU14764.B9C16R50
SLC39A8_JHU00847.B2C2R16
VPS13B_frag_JHU08730.B10C30R84
CD44_JHU02320.B2C5R42
DDO_JHU10674.B6C7R76
NOSIP_JHU01602.B1C12R26
TRIM21_JHU00287.B14C24R82
PSMC3IP_JHU11102.B7C13R82
KRT27_JHU17917.B14C22R66
C20orf24_JHU13221.B9C6R26
GDPD5_JHU01374.B1C25R20
RSU1_JHU00459.B2C12R12
BRK1_JHU01558.B1C5R28
GNRH1_JHU16860.B16C1R78
TPPP3_JHU02607.B1C10R40
RASSF7_JHU26008.B19C5R2
RSPO4_JHU17756.B15C21R60
IL10_JHU10600.B16C8R90
BARX2_JHU19610.B15C32R2
HGF_JHU10216.B16C8R86

Table 11 lists features found to be informative for distinguishing between (i) the presence of endometrial or ovarian cancer and (ii) the absence of endometrial or ovarian cancer. Each feature represents an abundance of a single autoantibody species that binds to the protein listed in a biological fluid. For instance, CCDC47_JHU18441.B16C10R86 refers to a log abundance of autoantibodies that bind to the human CCDC47 protein in a biological fluid. Age refers to the age of the subject and BMI refers to the body mass index of the subject.

In some embodiments, the first set of autoantibody species includes at least 3 autoantibody species. In some embodiments, each respective autoantibody species of the at least 3 autoantibody species specifically binds to a different molecular target selected from those listed in Table 11. In some embodiments, the first set of autoantibody species includes at least 5 autoantibody species. In some embodiments, each respective autoantibody species of the at least 5 autoantibody species specifically binds to a different molecular target selected from those listed in Table 11. In some embodiments, the first set of autoantibody species includes at least 10 autoantibody species. In some embodiments, each respective autoantibody species of the at least 10 autoantibody species specifically binds to a different molecular target selected from those listed in Table 11. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 11.

TABLE 11
Example features found to be informative for distinguishing
between (i) the presence of endometrial or ovarian cancer and
(ii) the absence of endometrial or ovarian cancer.
Example Feature

BMI
CCDC47_JHU18441.B16C10R86
CHRNA1_JHU04147.B2C18R66
GRID1_JHU06088.B6C18R2
PFN1_JHU14579.B9C26R50
HAUS4_JHU02028.B15C13R82
CD44_JHU02320.B2C5R42
ALDH2_JHU04131.B2C13R66
PLD3_JHU04101.B2C4R66
XCR1_JHU06622.B5C15R16
OR6C75_JHU13846.B9C12R42
ACVRL1_JHU04035.B2C4R62
TRPT1_JHU04502.B2C6R72
SPRR2E_JHU17584.B14C32R58
SERHL_JHU29987.B19C2R34
APOBEC3F_JHU14364.B9C19R44
OR10AD1_JHU08896.B18C20R16
MTUS1_JHU29795.B18C1R26
PRKCQ_JHU11774.B13C13R78
PIGO_JHU02758.B2C3R46
GABRA4_JHU05993.B5C19R6
MRGPRX2_JHU06561.B5C12R16
TMEM175_JHU04214.B2C10R64
CPSF7_JHU04072.B2C14R66
EMC1_JHU14211.B12C20R44
CANT1_JHU01276.B1C17R20
NIT1_JHU13649.B12C16R36
COG1_JHU16171.B9C32R74
BMI
CCL22_JHU03278.B2C6R52
RNMTL1_JHU00840.B1C15R14
MECP2_JHU14764.B9C16R50
TRAF3_JHU13778.B12C30R36
TPPP3_JHU02607.B1C10R40
ESCO1_JHU30374.B19C12R40
BRK1_JHU01558.B1C5R28
ASB15_JHU17968.B16C1R66
TRIM21_JHU00287.B14C24R82
FOXO3_JHU03298.B4C1R54
NOSIP_JHU01602.B1C12R26
BC104209_frag_JHU15715.B9C12R68

Table 12 lists features found to be informative for distinguishing between (i) a stage 3 or stage 4 endometrial or ovarian cancer and (ii) a stage 1 endometrial or ovarian cancer. Each feature represents an abundance of a single autoantibody species that binds to the protein listed in a biological fluid. For instance, TPRA1_JHU07039.B7C8R20 refers to a log abundance of autoantibodies that bind to the human TPRA1 protein in a biological fluid. Age refers to the age of the subject and BMI refers to the body mass index of the subject.

In some embodiments, the first set of autoantibody species includes at least 3 autoantibody species. In some embodiments, each respective autoantibody species of the at least 3 autoantibody species specifically binds to a different molecular target selected from those listed in Table 12. In some embodiments, the first set of autoantibody species includes at least 5 autoantibody species. In some embodiments, each respective autoantibody species of the at least 5 autoantibody species specifically binds to a different molecular target selected from those listed in Table 12. In some embodiments, the first set of autoantibody species includes at least 10 autoantibody species. In some embodiments, each respective autoantibody species of the at least 10 autoantibody species specifically binds to a different molecular target selected from those listed in Table 12. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 12.

TABLE 12
Example features found to be informative for distinguishing
between (i) a stage 3 or stage 4 endometrial or ovarian cancer
and (ii) a stage 1 endometrial or ovarian cancer.
Example Features

TPRA1_JHU07039.B7C8R20
KDELR1_JHU14121.B10C10R40
NOX1_JHU15727.B10C15R70
CLDN20_JHU03570.B15C20R76
PPA1_JHU18215.B14C12R44
SLC6A4_JHU12999.B9C31R22
TAGLN_JHU02383.B2C23R42
SLC7A10_JHU08911.B6C2R50
KLHDC7B_JHU12769.B12C32R20
ATP5C1_JHU06154.B7C12R8
LY6D_JHU13940.B12C32R38
CD28_JHU10959.B6C25R80
GPR15_JHU07138.B8C11R20
FEN1_JHU10212.B7C25R70
HLA-DRB1_JHU04553.B3C31R68
SLCO2B1_JHU06220.B7C26R12
C4orf19_JHU05577.B4C24R86
PLPP5_JHU15067.B10C14R56
CREG1_JHU19622.B16C29R2
CD36_JHU01460.B14C18R72
LINC01588_JHU29372.B18C1R24
NKD1_JHU06567.B8C12R18
HRK_JHU18687.B14C11R52
UGT3A2_JHU16574.B9C22R82
PCMT1_JHU14135.B14C21R8
CLDN16_JHU09520.B7C21R58
PTPMT1_JHU14043.B11C1R40
KPNA2_JHU16356.B12C10R76
TMBIM6_JHU06328.B8C9R12
KIR2DL4_JHU10222.B7C4R70
NPC2_JHU00340.B20C5R14
MAS1L_JHU05413.B3C26R86
KLC4_JHU10782.B8C15R80
EFCAB2_JHU02135.B3C6R32
CYP11A1_JHU08180.B7C29R40
ANKRD18DP_frag_JHU07248.B8C18R24
CCL22_JHU13032.B15C13R12
GPAA1_JHU14114.B10C21R38
COQ7_JHU13129.B12C32R28
ATPIF1_JHU03467.B1C26R56
ICAM4_JHU08865.B6C5R50
SENP5_JHU04111.B1C24R64
NAT16_JHU30154.B17C1R34
C20orf173_JHU30225.B17C22R42
TKTL1_JHU17398.B15C10R38
PGAP2_JHU14004.B20C20R14
CLUL1_JHU08462.B7C20R48
NIN_JHU30454.B19C22R42
EARS2_JHU13619.B10C24R32
GCLC_JHU13052.B11C12R30
SLC19A2_JHU14243.B10C3R48
OR10J5_JHU30052.B17C17R32
NETO1_JHU07745.B7C20R32
SOAT1_JHU13001.B15C32R88
C7orf43_JHU20814.B18C2R20
SLC30A7_JHU09867.B7C19R66
FAM71F2_JHU16205.B15C21R18
CFAP45_JHU19269.B17C32R18
ADD2_JHU09509.B7C30R60
SLC35A4_JHU08513.B7C12R46
C1orf43_JHU09234.B7C15R58
AGMAT_JHU08736.B8C18R44
GJB7_JHU06459.B6C28R10
TMEM208_JHU07526.B13C29R18
PRPSAP1_JHU00249.B4C25R6
CHRNB4_JHU30431.B17C23R38
MMD2_JHU13074.B11C3R30
AGER_JHU00677.B20C3R14
CDKAL1_JHU09807.B5C4R62
TIMMDC1_JHU00784.B1C12R14

Table 13 lists features found to be informative for distinguishing between (i) the presence of endometrial polyps and (ii) the absence of endometrial polyps. Each feature represents an abundance of a single autoantibody species that binds to the protein listed in a biological fluid. For instance, DYNC1H1_JHU16272.B12C19R78 refers to a log abundance of autoantibodies that bind to the human DYNC1H1 protein in a biological fluid. Age refers to the age of the subject and BMI refers to the body mass index of the subject.

In some embodiments, the first set of autoantibody species includes at least 3 autoantibody species. In some embodiments, each respective autoantibody species of the at least 3 autoantibody species specifically binds to a different molecular target selected from those listed in Table 13. In some embodiments, the first set of autoantibody species includes at least 5 autoantibody species. In some embodiments, each respective autoantibody species of the at least 5 autoantibody species specifically binds to a different molecular target selected from those listed in Table 13. In some embodiments, the first set of autoantibody species includes at least 10 autoantibody species. In some embodiments, each respective autoantibody species of the at least 10 autoantibody species specifically binds to a different molecular target selected from those listed in Table 13. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 13.

TABLE 13
Example features found to be informative for distinguishing
between (i) the presence of endometrial polyps and (ii) the
absence of endometrial polyps.
Example Features

DYNC1H1_JHU16272.B12C19R78
MCAT_JHU12114.B12C19R10
IGSF21_JHU09355.B7C8R56
AKAP10_JHU00008.B14C7R14
IFT122_JHU18567.B14C26R52
TAT_JHU28032.B20C19R10
DIAPH3_JHU06841.B14C27R16
TCL1A_JHU04883.B15C16R8
GNG7_JHU03885.B4C23R64
CCDC167_JHU30299.B19C7R36
C16orf13_JHU03987.B4C22R62
WASF2_JHU05846.B5C12R4
TDO2_JHU05459.B1C20R90
SLC16A2_JHU18127.B14C31R64
S100A7_JHU04203.B4C21R64
MTA2_JHU12215.B12C18R8
SCARB2_JHU01220.B16C32R8
HOXA5_JHU02920.B4C4R46
EPHA4_JHU16407.B12C23R84
SWT1_JHU04042.B4C24R64
UBE2F_JHU01145.B3C28R14
AGTR1_JHU16058.B4C14R66
MFAP3L_JHU06958.B7C5R24
LINC00846_JHU01645.B4C29R30
MAFB_JHU12972.B12C13R24
ACTRT1_JHU01734.B4C16R28
LITAF_JHU15054.B12C27R56
ZDHHC15_JHU11319.B8C29R88
CALHM3_JHU05885.B5C22R4
SLC16A5_JHU08615.B7C8R44
PSMB7_JHU02562.B4C24R40
MRPL35_JHU14955.B12C23R60
GPR63_JHU16089.B12C26R78
ATP5F1_JHU01925.B4C23R36
GABRA4_JHU05993.B5C19R6
SLC6A16_JHU13291.B12C31R88
CILP_JHU18348.B16C24R46
ETV3_JHU01856.B1C7R30
DNM1L_JHU05395.B4C15R86
OVGP1_JHU19124.B16C27R20
XRCC4_JHU02111.B15C17R76
LPIN1_JHU08594.B5C9R46
TBRG1_JHU04020.B4C29R62
RABL2B_JHU00263.B4C25R2
PNPLA4_JHU04861.B4C25R78
CLDN18_JHU11246.B5C22R90
MTFMT_JHU04278.B4C18R62
TMEM87A_JHU15184.B12C1R58
TAF6_JHU04019.B4C17R64
DNM3_JHU17340.B15C18R42
TGIF2LY_JHU16776.B16C4R42
TAMM41_JHU00885.B4C32R14
GLI4_JHU05601.B4C29R12
SERPINE1_JHU01324.B20C5R18
CCT3_JHU02124.B1C11R34
PARM1_JHU04438.B4C24R68
BMX_JHU00106.B3C29R2

Table 14 lists features found to be informative for distinguishing between (i) the presence of adenomyosis and (ii) the absence of adenomyosis. Each feature represents an abundance of a single autoantibody species that binds to the protein listed in a biological fluid. For instance, DOK6_JHU10965.B7C19R82 refers to a log abundance of autoantibodies that bind to the human DOK6 protein in a biological fluid. Age refers to the age of the subject and BMI refers to the body mass index of the subject.

In some embodiments, the first set of autoantibody species includes at least 3 autoantibody species. In some embodiments, each respective autoantibody species of the at least 3 autoantibody species specifically binds to a different molecular target selected from those listed in Table 14. In some embodiments, the first set of autoantibody species includes at least 5 autoantibody species. In some embodiments, each respective autoantibody species of the at least 5 autoantibody species specifically binds to a different molecular target selected from those listed in Table 14. In some embodiments, the first set of autoantibody species includes at least 10 autoantibody species. In some embodiments, each respective autoantibody species of the at least 10 autoantibody species specifically binds to a different molecular target selected from those listed in Table 14. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 14.

TABLE 14
Example features found to be informative for
distinguishing between (i) the presence of
adenomyosis and (ii) the absence of adenomyosis.
Example Features

DOK6_JHU10965.B7C19R82
IFFO1_JHU13149.B9C32R30
ASB15_JHU17968.B16C1R66
SHMT1_JHU05535.B3C9R90
TJP1_JHU18598.B16C23R50
ASB15_JHU17968.B13C6R74
MKS1_frag_JHU13540.B12C27R34
MDM2_JHU11560.B11C5R2
EGFL7_JHU14827.B12C18R54
IL18R1_JHU13737.B12C28R34
SPOCK1_JHU05542.B3C29R88
LCAT_JHU01096.B4C28R14
GAB2_JHU14748.B11C19R54
SOX30_JHU10814.B7C15R82
BRE_JHU13508.B12C17R32
HES2_JHU13344.B12C31R30
FBXO38_JHU12285.B12C3R14
SNX12_JHU14788.B10C6R54
NINJ1_JHU02068.B3C8R36
FBXO25_JHU05890.B5C8R6
TTN_JHU15762.B12C3R70
LDB3_JHU19998.B18C19R8
MTM1_JHU04093.B2C25R64
TNNI3K_JHU15663.B12C10R62
POGLUT1_JHU03112.B16C25R72
DYNC2LI1_JHU07224.B7C28R20
ENO2_JHU00604.B4C25R10
SCAMP3_JHU01123.B1C21R14
SPTLC2_JHU09005.B5C9R54
BANK1_JHU18156.B13C26R48
C19orf52_JHU08268.B5C24R40
SPEG_JHU03824.B3C21R58
WSCD1_JHU00764.B2C31R10
OLA1_JHU13444.B9C12R32
RPS27A_JHU00359.B14C1R80
ZDHHC19_JHU04317.B2C5R68
PAK2_JHU15639.B12C7R64
OGG1_JHU14771.B12C24R52
RND2_JHU10913.B7C29R82
NCR1_JHU16314.B10C25R78
ANKRD26P1_JHU11355.B8C6R90
MYBPH_JHU07744.B8C5R32
POR_JHU04102.B2C11R64
MTM1_JHU04093.B2C17R66
CHGA_JHU08656.B8C24R48
SPACA7_JHU19056.B16C2R20
C15orf57_JHU11437.B7C21R90
ALG9_JHU14268.B12C17R44
CHRM1_JHU14095.B12C30R40
FN3KRP_JHU00316.B4C13R2
GALNTL5_JHU14201.B11C2R44
AIFM3_JHU09220.B5C5R56
PIK3CA_JHU11201.B14C4R16
PPP1R27_JHU14736.B12C1R54
ZFYVE16_JHU08734.B14C32R74
CPB2_JHU13902.B12C27R42
TSPAN7_JHU07481.B7C17R28
GTF3C2_JHU14305.B12C22R46
PAXIP1_frag_JHU13364.B9C10R28
SERPINF2_JHU04205.B1C10R66
CTDSP1_JHU07422.B7C29R28
GM2A_JHU14647.B12C22R54
PAX3_JHU13546.B12C27R36
SEC14L2_JHU20221.B19C25R8
VAMP1_JHU10734.B7C18R78
FAM189B_JHU08652.B7C11R46
SERPINB10_JHU11493.B7C13R88
PNLIPRP3_JHU16983.B15C28R30
ANKRD45_JHU11235.B8C17R88
YS049_JHU03360.B2C16R54
VWA2_JHU19351.B14C18R24
LINC01104_frag_JHU11272.B6C9R88
CHST3_JHU16496.B10C8R80
KJ903660_JHU15199.B10C30R58
TWSG1_JHU01052.B1C19R16
C6orf62_JHU05971.B7C6R6
POFUT2_JHU09766.B7C27R64
CDK1_JHU04433.B2C11R68
SLC18A3_JHU09196.B7C31R50
TMEM132B_JHU17101.B13C15R34
CTXN1_JHU11247.B6C9R86
DHRS1_JHU01466.B2C30R20
CPO_JHU19082.B15C17R22
LACTB_JHU15523.B12C11R64
TRMU_JHU15188.B12C19R60
GALC_JHU12861.B12C11R24
ZBTB10_JHU29387.B20C19R20
MRPL30_JHU03229.B13C12R18
SLC9B2_JHU07722.B8C20R32
AC209618.3_frag_JHU14745.B12C19R52
TPSAB1_JHU14797.B12C6R50
ATP6VOD1_JHU14084.B9C21R40
FKBP10_JHU05028.B3C26R84
UBB_JHU14256.B9C12R48
B4GALNT2_JHU16925.B14C13R28
DEFB109P1_JHU18757.B13C28R50
SEPSECS_JHU06239.B6C26R10
ZNF695_JHU06716.B6C32R14
VAMP4_JHU02107.B3C29R32
RSRP1_JHU15494.B12C19R62
NME7_JHU11670.B9C9R6
RHOJ_JHU11489.B6C7R86
NDUFA13_JHU06881.B7C30R14
ACSF3_JHU16598.B16C12R84
RAB35_JHU00255.B3C19R2
RAD51C_JHU14491.B12C18R44
LRRC4_JHU29385.B18C19R24
Capn15_JHU19944.B16C26R12
CEP72_JHU08554.B8C9R44
ICAM4_JHU07437.B5C27R30
CPA5_JHU05490.B2C9R88
LOC401040_JHU14744.B12C25R52
LYNX1_JHU11558.B12C15R6
EDIL3_JHU13913.B11C31R86
DGUOK_JHU00002.B16C10R74
PHF10_JHU21937.B20C31R14
RPS27A_JHU16229.B12C9R78
P2RY4_JHU15925.B9C16R88
NCF4_JHU10904.B7C32R80
CHRDL1_JHU03092.B2C5R50
ZBTB25_JHU05850.B5C12R2
IFNLR1_JHU11179.B8C12R90
KLHDC9_JHU15417.B12C9R64
GPATCH1_JHU08475.B6C26R46
EDIL3_JHU06744.B6C6R14
MEF2C_JHU13353.B11C13R26
PPP2R2A_JHU13277.B12C19R28
AC017104.8_frag_JHU11756.B12C1R6

Table 15 lists features found to be informative for distinguishing between (i) the presence of leiomyoma and (ii) the absence of leiomyoma. Each feature represents an abundance of a single autoantibody species that binds to the protein listed in a biological fluid. For instance, DOK6_JHU10965.B7C19R82 refers to a log abundance of autoantibodies that bind to the human DOK6 protein in a biological fluid. Age refers to the age of the subject and BMI refers to the body mass index of the subject.

In some embodiments, the first set of autoantibody species includes at least 3 autoantibody species. In some embodiments, each respective autoantibody species of the at least 3 autoantibody species specifically binds to a different molecular target selected from those listed in Table 15. In some embodiments, the first set of autoantibody species includes at least 5 autoantibody species. In some embodiments, each respective autoantibody species of the at least 5 autoantibody species specifically binds to a different molecular target selected from those listed in Table 15. In some embodiments, the first set of autoantibody species includes at least 10 autoantibody species. In some embodiments, each respective autoantibody species of the at least 10 autoantibody species specifically binds to a different molecular target selected from those listed in Table 15. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 15.

TABLE 15
Example features found to be informative for
distinguishing between (i) the presence of leiomyoma
and (ii) the absence of leiomyoma.
Example Features

HOXB4_JHU16744.B13C11R42
PTGES2_JHU11680.B12C22R2
HOXC13_JHU16669.B13C8R38
FOXL1_JHU19635.B13C22R2
CADPS_JHU01840.B4C21R26
ITK_JHU15712.B9C12R70
DNAJC18_JHU04341.B4C17R68
RTP4_JHU01042.B4C19R18
CCDC93_JHU15967.B9C18R72
HAX1_JHU08008.B6C21R32
BCCIP_JHU13890.B14C28R10
HOXD12_JHU16655.B13C22R38
GNG3_JHU18269.B15C9R46
THAP1_JHU03633.B1C29R60
CHMP1A_JHU18835.B13C15R68
ZNF547_JHU11988.B9C26R10
ZNF57_JHU13016.B9C30R24
Repin1_JHU19770.B14C16R4
RHOB_JHU09670.B7C3R64
EED_JHU26265.B19C3R6
CDX1_JHU16635.B13C8R42
RILP_JHU08133.B8C5R42
NENF_JHU08793.B4C23R68
UBE2J2_JHU14896.B16C7R16
ACSL5_JHU11616.B12C14R4
RIPK4_JHU17274.B15C12R52
VIM_JHU03068.B4C18R48
PRR19_JHU16312.B9C16R78
SH2D1A_JHU12232.B11C28R12
ATG14_JHU29809.B17C7R36
HOXA13_JHU16670.B14C27R42
ACSS1_JHU12256.B12C20R18
OR52K2_JHU30068.B17C8R34
MIB2_JHU17358.B13C28R42
ZNF337_JHU16793.B13C16R38
HESX1_JHU09729.B13C16R12

In some embodiments, for each autoantibody species in the first set of autoantibody species, the corresponding abundance value for the respective autoantibody species includes an abundance of IgG and IgA homologues of the first set of autoantibody species in the biological fluid sample. In some embodiments, the IgG and IgA profiles are combined, thereby determining the respective abundance level of each autoantibody in the plurality of autoantibodies. In some embodiments, only one of either of the IgG or IgA profiles is used.

Referring to block 1407, method 1400 includes using the autoantibody abundance dataset to determine values for each of a first set of autoantibody abundance features, thereby obtaining a first feature dataset for the subject. As described herein, in some embodiments, the autoantibody abundance features are abundance values for autoantibodies species, logs of the autoantibody abundance values, or a normalized abundance value thereof. For instance, in some embodiments, a normalization technique is applied to the autoantibody abundance values or logs thereof, such as scaling to a range, clipping, log scaling, or determining a z-score.

In some embodiments, the first set of autoantibody abundance features includes at least 5 of the features listed in Table 2. In some embodiments, the first set of protein abundance features includes at least 10 of the features listed in Table 2. In some embodiments, the first set of protein abundance features includes at least 25 of the features listed in Table 2. In some embodiments, the first set of protein abundance features includes at least 50 of the features listed in Table 2. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or all 118 of the features listed in Table 2.

In some embodiments, the first set of autoantibody abundance features includes at least 5 of the features listed in Table 3. In some embodiments, the first set of protein abundance features includes at least 10 of the features listed in Table 3. In some embodiments, the first set of protein abundance features includes at least 25 of the features listed in Table 3. In some embodiments, the first set of protein abundance features includes at least 50 of the features listed in Table 3. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or all 106 of the features listed in Table 3.

In some embodiments, the first set of autoantibody abundance features includes at least 5 of the features listed in Table 4. In some embodiments, the first set of protein abundance features includes at least 10 of the features listed in Table 4. In some embodiments, the first set of protein abundance features includes at least 25 of the features listed in Table 4. In some embodiments, the first set of protein abundance features includes at least 50 of the features listed in Table 4. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, or all 122 of the features listed in Table 4.

In some embodiments, the first set of autoantibody abundance features includes at least 5 of the features listed in Table 5. In some embodiments, the first set of protein abundance features includes at least 10 of the features listed in Table 5. In some embodiments, the first set of protein abundance features includes at least 25 of the features listed in Table 5. In some embodiments, the first set of protein abundance features includes at least 50 of the features listed in Table 5. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, or all 154 of the features listed in Table 5.

In some embodiments, the first set of autoantibody abundance features includes at least 5 of the features listed in Table 6. In some embodiments, the first set of protein abundance features includes at least 10 of the features listed in Table 6. In some embodiments, the first set of protein abundance features includes at least 25 of the features listed in Table 6. In some embodiments, the first set of protein abundance features includes at least 50 of the features listed in Table 6. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, or all 152 of the features listed in Table 6.

In some embodiments, the first set of autoantibody abundance features includes at least 5 of the features listed in Table 7. In some embodiments, the first set of protein abundance features includes at least 10 of the features listed in Table 7. In some embodiments, the first set of protein abundance features includes at least 25 of the features listed in Table 7. In some embodiments, the first set of protein abundance features includes at least 50 of the features listed in Table 7. In some embodiments, the first set of protein abundance features includes at least 2, 3, 4, 5, 6, 7, 8, 9, 10, 11, 12, 13, 14, 15, 16, 17, 18, 19, 20, 21, 22, 23, 24, 25, 26, 27, 28, or all 29 of the features listed in Table 7.

In some embodiments, the first set of autoantibody abundance features includes at least 5 of the features listed in Table 8. In some embodiments, the first set of protein abundance features includes at least 10 of the features listed in Table 8. In some embodiments, the first set of protein abundance features includes at least 25 of the features listed in Table 8. In some embodiments, the first set of protein abundance features includes at least 50 of the features listed in Table 8. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, or all 132 of the features listed in Table 8.

In some embodiments, the first set of autoantibody abundance features includes at least 5 of the features listed in Table 9. In some embodiments, the first set of protein abundance features includes at least 10 of the features listed in Table 9. In some embodiments, the first set of protein abundance features includes at least 25 of the features listed in Table 9. In some embodiments, the first set of protein abundance features includes at least 50 of the features listed in Table 9. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, or all 112 of the features listed in Table 9.

In some embodiments, the first set of autoantibody abundance features includes at least 5 of the features listed in Table 10. In some embodiments, the first set of protein abundance features includes at least 10 of the features listed in Table 10. In some embodiments, the first set of protein abundance features includes at least 25 of the features listed in Table 10. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, or all 41 of the features listed in Table 10.

In some embodiments, the first set of autoantibody abundance features includes at least 5 of the features listed in Table 11. In some embodiments, the first set of protein abundance features includes at least 10 of the features listed in Table 11. In some embodiments, the first set of protein abundance features includes at least 25 of the features listed in Table 11. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, or all 41 of the features listed in Table 11.

In some embodiments, the first set of autoantibody abundance features includes at least 5 of the features listed in Table 12. In some embodiments, the first set of protein abundance features includes at least 10 of the features listed in Table 12. In some embodiments, the first set of protein abundance features includes at least 25 of the features listed in Table 12. In some embodiments, the first set of protein abundance features includes at least 50 of the features listed in Table 12. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, or all 70 of the features listed in Table 12.

In some embodiments, the first set of autoantibody abundance features includes at least 5 of the features listed in Table 13. In some embodiments, the first set of protein abundance features includes at least 10 of the features listed in Table 13. In some embodiments, the first set of protein abundance features includes at least 25 of the features listed in Table 13. In some embodiments, the first set of protein abundance features includes at least 50 of the features listed in Table 13. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, or all 57 of the features listed in Table 13.

In some embodiments, the first set of autoantibody abundance features includes at least 5 of the features listed in Table 14. In some embodiments, the first set of protein abundance features includes at least 10 of the features listed in Table 14. In some embodiments, the first set of protein abundance features includes at least 25 of the features listed in Table 14. In some embodiments, the first set of protein abundance features includes at least 50 of the features listed in Table 14. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, or all 128 of the features listed in Table 14.

In some embodiments, the first set of autoantibody abundance features includes at least 5 of the features listed in Table 15. In some embodiments, the first set of protein abundance features includes at least 10 of the features listed in Table 15. In some embodiments, the first set of protein abundance features includes at least 25 of the features listed in Table 15. In some embodiments, the first set of protein abundance features includes at least 2, 3, 4, 5, 6, 7, 8, 9, 10, 11, 12, 13, 14, 15, 16, 17, 18, 19, 20, 25, 30, 35, or all 36 of the features listed in Table 15.

Referring to block 1408, method 1400 includes inputting the first feature dataset into a classifier trained to distinguish between at least two states of the gynecological disorder based on at least abundance values for the first set of autoantibody species, thereby obtaining a probability or likelihood from the classifier that the subject has a particular state the gynecological disorder. As described above, many types of classifiers can be used in conjunction with the methods described herein.

In some embodiments, the classifier determines a disease profile Vₛ for the subject including a weighted sum W of the respective abundance values in the first autoantibody abundance dataset. W is calculated as:

Wₛ=Σᵢ₌₁^{m}(AᵢEᵢ),

where Eᵢ is a value of a respective autoantibody abundance feature i, in the first feature dataset m autoantibody abundance features, determined for the autoantibody abundance dataset, and Aᵢ is a weight for autoantibody abundance feature i.

In some embodiments, for each respective autoantibody abundance feature i in the first set of m autoantibody abundance features, the weight Aᵢ is calculated as:

Aᵢ˜Dᵢ⁻¹Σⱼ₌₁^{k}([Cᵢⱼ]⁻¹Zⱼ),

where Dᵢ is the standard deviation of the value of autoantibody abundance feature i in a training set of biological fluid samples. The training set includes a first subset of biological fluid samples from training subjects having a first state of the gynecological disorder, and a second subset of biological fluid samples from training subjects having a second state of the gynecological disorder. [Cᵢⱼ [symbol] is a matrix of pairwise correlation between the values of autoantibody abundance features i and j in the first training set, such that [Cᵢⱼ]⁻¹ is the reciprocal matrix of pairwise correlation, where k=m−1, and Zⱼ is a z-score for the values of autoantibody abundance feature j in the first training set. Zⱼ is calculated as:

$${Z}_{j} = \frac{{{( {E}_{j} 〉}_{1} - ( {E}_{j} 〉}_{2}}{{D}_{j}} ,$$

where [symbol] Eⱼ [symbol] ₁ is the average value of autoantibody abundance feature j determined for the first subset of biological fluid samples, [symbol] Eⱼ [symbol] ₂ is the average value of autoantibody abundance feature j determined for the second subset of biological fluid samples, and Dⱼ is the standard deviation of the values of autoantibody abundance feature j in the training set of biological fluid samples.

In some embodiments, the classifier was trained to distinguish between the at least two states of the ovarian or uterine disease condition based on at least abundance values for the first set of autoantibody species and one or more secondary features of the subject.

In some embodiments, the ovarian or uterine disease condition is an ovarian cancer or an endometrial cancer. The one or more secondary features of the subject include two or more of the features selected from the group consisting of an age of the subject, a pregnancy history of the subject, a breastfeeding history of the subject, a BRCA1 genotype of the subject, a BRCA2 genotype of the subject, a breast cancer history of the subject, and a familial history of endometrial cancer, ovarian cancer, or breast cancer.

In some embodiments, the method further includes obtaining a second biological sample from the subject. The method includes determining a plurality of secondary features from the second biological sample, thereby obtaining a secondary feature dataset for the subject. The method includes inputting the secondary feature dataset into the classifier.

In some embodiments, the classifier was trained to distinguish between (i) the presence of an ovarian cancer or uterine cancer and (ii) the absence of the ovarian cancer or the uterine cancer. The method further includes, when the probability or likelihood obtained from the classifier indicates that the subject has the ovarian cancer or the uterine cancer, administering a therapy for the ovarian cancer or the uterine cancer to the subject. The method also includes, when the probability or likelihood obtained from the classifier indicates that the subject does not have the ovarian cancer or the uterine cancer, forgoing administration of the therapy for the ovarian cancer or the uterine cancer to the subject.

In some embodiments, the classifier was trained to distinguish between (i) a first stage of an ovarian cancer or uterine cancer and (ii) a second stage of the ovarian cancer or the uterine cancer that is more advanced than the first stage of the ovarian cancer or the uterine cancer. The method further includes, when the probability or likelihood obtained from the classifier indicates that the subject has the first stage of the ovarian cancer or the uterine cancer, administering a first therapy for the ovarian cancer or the uterine cancer to the subject. The method also includes, when the probability or likelihood obtained from the classifier indicates that the subject has the first stage of the ovarian cancer or the uterine cancer, administering a second therapy for the ovarian cancer or the uterine cancer to the subject.

In some embodiments, the classifier was trained to distinguish between (i) the presence of adenomyosis, endometrial polyps, leiomyoma, or endometriosis and (ii) the absence of the adenomyosis, endometrial polyps, leiomyoma, or endometriosis. The method further includes, when the probability or likelihood obtained from the classifier indicates that the subject has the adenomyosis, endometrial polyps, leiomyoma, or endometriosis, administering a therapy for the adenomyosis, endometrial polyps, leiomyoma, or endometriosis to the subject. The method also includes, when the probability or likelihood obtained from the classifier indicates that the subject does not have the adenomyosis, endometrial polyps, leiomyoma, or endometriosis, forgoing administration of the therapy for the adenomyosis, endometrial polyps, leiomyoma, or endometriosis to the subject.

Referring to block 1502 of FIG. 15, a method is provided for evaluating a gynecological disorder in a subject. In some embodiments, the gynecological disorder is an ovarian cancer or an endometrial cancer. In some embodiments, the gynecological disorder is adenomyosis, endometrial polyps, leiomyoma, or endometriosis (e.g., complex atypical hyperplasia and/or an atrophic endometrium and/or an endometrial thickening).

In some embodiments, the method evaluates a subject for a disease condition. In some such embodiments, the disease condition comprises a non-cancerous condition. In some embodiments, the non-cancerous condition is endometriosis, tuberculosis, fungal infections, or bacterial pneumonias. See Radha et al. et al. 2014 J Cytol. 31(3), 136-138. In some embodiments, the non-cancerous condition is pericoronitis, hematemesis, ulcerative colitis, ulcer, osteoarthritis, sinusitis, or other conditions known in the art.

In some such embodiments, the disease condition comprises a pre-cancerous or cancer condition. A pre-cancerous disease condition involves abnormal cells that are at an increased risk of developing into cancer. In some embodiments, the cancer condition comprises endometrial cancer, ovarian cancer, cervical cancer, uterine sarcoma, vaginal cancer, vulvar cancer, gestational trophoblastic disease, or other reproductive cancer. In some embodiments, the cancer condition comprises breast cancer, esophageal cancer, lung cancer, renal cancer, colorectal cancer, nasopharyngeal cancer, lymphoma, or any other cancer condition known in the art.

In some embodiments, the stage of endometrial cancer comprises stage 0 endometrial cancer (e.g., complex atypical hyperplasia), stage IA endometrial cancer, stage IB endometrial cancer, stage II endometrial cancer, stage III endometrial cancer, or stage IV endometrial cancer. In some embodiments, the stage of ovarian cancer comprises stage 0 ovarian cancer, stage IA ovarian cancer, stage IB ovarian cancer, stage II ovarian cancer, stage III ovarian cancer, or stage IV ovarian cancer.

In some embodiments, the subject is asymptomatic for endometrial cancer. In some embodiments, the subject is asymptomatic for ovarian and/or endometrial cancer. In some embodiments, subjects are asymptomatic for endometrial cancer but do exhibit complex atypical hyperplasia (CAH). This is a pre-cancerous state (e.g., equivalent to stage 0 endometrial cancer) that is associated with an approximately 40% increased risk of a subject developing endometrial cancer. See e.g., Suh-Burgmann et al. et al. 2009 Obstetrics and Gynecology 114(3), 523-529. In some embodiments, the subject is symptomatic for ovarian and/or endometrial cancer. In some embodiments, a subject is from a population with an increased risk for ovarian and/or endometrial cancer. In some embodiments, the increased risk is that the subject has Lynch syndrome, the subject is obese, the subject has family history of ovarian and/or endometrial cancer, the subject has a BRCA mutation, and/or the subject is over a predetermined age—e.g., where the predetermined age is at least 40, at least 45, at least 50, at least 55, at least 60, at least 65, or at least 70 years of age). In some embodiments, the subject is asymptomatic. In some embodiments, the subject is experiencing pelvic pain, abnormal bleeding, or infertility.

In some embodiments, a subject is concurrently evaluated for a stage of an additional cancer condition distinct from ovarian and endometrial cancer. In some embodiments, another cancer condition is selected from the group consisting of lung cancer, prostate cancer, colorectal cancer, renal cancer, cancer of the esophagus, cervical cancer, bladder cancer, gastric cancer, nasopharyngeal cancer, or a combination thereof.

Referring to block 1504, the evaluation method proceeds by obtaining a biological fluid sample, e.g., a blood plasma or uterine lavage fluid, from the subject. In some embodiments, a uterine lavage fluid is collected from the subject via hysteroscopy combined with curettage. In some embodiments, uterine lavage fluid is collected from the subject via uterine washings.

In some embodiments, a second biological fluid is collected from the subject. In some embodiments, the second biological fluid is a lavage fluid. In some embodiments, the lavage fluid sample is a bronchoalveolar lavage fluid sample, a gastric lavage fluid sample, a ductal lavage fluid sample, a nasal irrigation sample, a peritoneal lavage fluid sample, a peritoneal lavage fluid sample, an arthroscopic lavage fluid sample, or ear lavage fluid sample. In some embodiments, the second biological fluid is blood or a fraction thereof, such as a blood plasma fraction.

In some embodiments, a body cavity from which the lavage fluid sample is collected determines which type(s) of cancer said lavage fluid sample is assayed for (e.g., bladder cancer, oral cancer, lung cancer, gastrointestinal cancer, endometrial, and/or ovarian). In some such embodiments, the method further evaluates the subject for a stage of bladder cancer, a stage of oral cancer, a stage of lung cancer, a stage of gastrointestinal cancer, a stage of endometrial cancer, and/or a stage of ovarian cancer, respectively.

Referring to block 1506, the evaluation method continues by determining, for each autoantibody species in a plurality of autoantibody species, a corresponding abundance value for the respective autoantibody species in the biological fluid sample. The method thereby includes obtaining a master autoantibody abundance dataset for the subject.

In some embodiments, for each autoantibody species in the first set of autoantibody species, the corresponding abundance value for the respective autoantibody species includes an abundance of IgG and IgA homologues of the first set of autoantibody species in the biological fluid sample. In some embodiments, the IgG and IgA profiles are combined, thereby determining the respective abundance level of each autoantibody in the plurality of autoantibodies. In some embodiments, only one of either of the IgG or IgA profiles is used.

In some embodiments, the first set of autoantibody species includes at least 3 autoantibody species. In some embodiments, each respective autoantibody species of the at least 3 autoantibody species specifically binds to a different molecular target selected from those listed in Table 3. In some embodiments, the first set of autoantibody species includes at least 5 autoantibody species. In some embodiments, each respective autoantibody species of the at least 5 autoantibody species specifically binds to a different molecular target selected from those listed in Table 3. In some embodiments, the first set of autoantibody species includes at least 10 autoantibody species. In some embodiments, each respective autoantibody species of the at least 10 autoantibody species specifically binds to a different molecular target selected from those listed in Table 3. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 3.

In some embodiments, the first set of autoantibody species includes at least 3 autoantibody species. In some embodiments, each respective autoantibody species of the at least 3 autoantibody species specifically binds to a different molecular target selected from those listed in Table 3. In some embodiments, the first set of autoantibody species includes at least 5 autoantibody species. In some embodiments, each respective autoantibody species of the at least 5 autoantibody species specifically binds to a different molecular target selected from those listed in Table 3. In some embodiments, the first set of autoantibody species includes at least 10 autoantibody species. In some embodiments, each respective autoantibody species of the at least 10 autoantibody species specifically binds to a different molecular target selected from those listed in Table 3. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 3.

In some embodiments, the first set of autoantibody species includes at least 3 autoantibody species. In some embodiments, each respective autoantibody species of the at least 3 autoantibody species specifically binds to a different molecular target selected from those listed in Table 4. In some embodiments, the first set of autoantibody species includes at least 5 autoantibody species. In some embodiments, each respective autoantibody species of the at least 5 autoantibody species specifically binds to a different molecular target selected from those listed in Table 4. In some embodiments, the first set of autoantibody species includes at least 10 autoantibody species. In some embodiments, each respective autoantibody species of the at least 10 autoantibody species specifically binds to a different molecular target selected from those listed in Table 4. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 4.

In some embodiments, the first set of autoantibody species includes at least 3 autoantibody species. In some embodiments, each respective autoantibody species of the at least 3 autoantibody species specifically binds to a different molecular target selected from those listed in Table 5. In some embodiments, the first set of autoantibody species includes at least 5 autoantibody species. In some embodiments, each respective autoantibody species of the at least 5 autoantibody species specifically binds to a different molecular target selected from those listed in Table 5. In some embodiments, the first set of autoantibody species includes at least 10 autoantibody species. In some embodiments, each respective autoantibody species of the at least 10 autoantibody species specifically binds to a different molecular target selected from those listed in Table 5. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 5.

In some embodiments, the first set of autoantibody species includes at least 3 autoantibody species. In some embodiments, each respective autoantibody species of the at least 3 autoantibody species specifically binds to a different molecular target selected from those listed in Table 6. In some embodiments, the first set of autoantibody species includes at least 5 autoantibody species. In some embodiments, each respective autoantibody species of the at least 5 autoantibody species specifically binds to a different molecular target selected from those listed in Table 6. In some embodiments, the first set of autoantibody species includes at least 10 autoantibody species. In some embodiments, each respective autoantibody species of the at least 10 autoantibody species specifically binds to a different molecular target selected from those listed in Table 6. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 6.

In some embodiments, the first set of autoantibody species includes at least 3 autoantibody species. In some embodiments, each respective autoantibody species of the at least 3 autoantibody species specifically binds to a different molecular target selected from those listed in Table 7. In some embodiments, the first set of autoantibody species includes at least 5 autoantibody species. In some embodiments, each respective autoantibody species of the at least 5 autoantibody species specifically binds to a different molecular target selected from those listed in Table 7. In some embodiments, the first set of autoantibody species includes at least 10 autoantibody species. In some embodiments, each respective autoantibody species of the at least 10 autoantibody species specifically binds to a different molecular target selected from those listed in Table 7. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 7.

In some embodiments, the plurality of autoantibody species includes at least 5 autoantibody species. Each respective autoantibody species of the at least 5 autoantibody species binds to a molecular target in a different pathway or cell type signature selected from those listed in Table 1.

Referring to block 1508, the evaluation method continues by inputting a first subset of the master autoantibody abundance dataset into a first classifier. The first classifier is trained to distinguish between the presence of adenomyosis and the absence of adenomyosis based on at least abundance values for a first subset of the plurality of autoantibody species. The method thereby includes obtaining a probability or likelihood from the classifier that the subject has adenomyosis.

Referring to block 1510, the evaluation method continues by inputting a second subset of the master autoantibody abundance dataset into a second classifier. The second classifier is trained to distinguish between the presence of endometrial polyps and the absence of endometrial polyps based on at least abundance values for a second subset of the plurality of autoantibody species. The method thereby includes obtaining a probability or likelihood from the classifier that the subject has endometrial polyps.

Referring to block 1512, the evaluation method continues by inputting a third subset of the master autoantibody abundance dataset into a third classifier. The third classifier is trained to distinguish between the presence of leiomyoma and the absence of leiomyoma based on at least abundance values for a third subset of the plurality of autoantibody species. The method thereby includes obtaining a probability or likelihood from the classifier that the subject has leiomyoma.

Referring to block 1514, the evaluation method inputs a fourth subset of the master autoantibody abundance dataset into a fourth classifier. The fourth classifier is trained to distinguish between the presence of endometriosis and the absence of endometriosis based on at least abundance values for a fourth subset of the plurality of autoantibody species. The method thereby includes obtaining a probability or likelihood from the classifier that the subject has endometriosis.

In some embodiments of method 1500, the classifier uses the autoantibody abundance dataset to determine values for each of a first set of autoantibody abundance features, which are used in the classification process, e.g., at steps 1508-1514. As described herein, in some embodiments, the autoantibody abundance features are abundance values for autoantibodies species, logs of the autoantibody abundance values, or a normalized abundance value thereof. For instance, in some embodiments, a normalization technique is applied to the autoantibody abundance values or logs thereof, such as scaling to a range, clipping, log scaling, or determining a z-score.

In some embodiments, the method further includes, when the probability or likelihood obtained from the first classifier indicates that the subject has adenomyosis, administering a therapy for adenomyosis to the subject. The method includes, when the probability or likelihood obtained from the second classifier indicates that the subject has endometrial polyps, administering a therapy for endometrial polyps to the subject. The method includes, when the probability or likelihood obtained from the third classifier indicates that the subject has leiomyoma, administering a therapy for leiomyoma to the subject. The method includes, when the probability or likelihood obtained from the fourth classifier indicates that the subject has endometriosis, administering a therapy for endometriosis to the subject. The method also includes, when the probabilities or likelihoods obtained from the first through fourth classifiers indicates that the subject does not have at least one condition selected from the group consisting of adenomyosis, endometrial polyps, leiomyoma, and endometriosis, forgoing administration of the therapies for adenomyosis, endometrial polyps, leiomyoma, and endometriosis.

In some embodiments, the method further includes, when the probabilities or likelihoods obtained from the first through fourth classifiers indicates that the subject has at least one condition selected from the group consisting of adenomyosis, endometrial polyps, leiomyoma, and endometriosis, confirming a diagnosis for the at least one condition selected from the group consisting of adenomyosis, endometrial polyps, leiomyoma, and endometriosis. The confirming is performed by further clinical evaluation, prior to administering the therapy for the at least one condition selected from the group consisting of adenomyosis, endometrial polyps, leiomyoma, and endometriosis to the subject.

In some embodiments, the method further includes inputting a fifth subset of the master autoantibody abundance dataset into a fifth classifier trained to distinguish between the presence of an ovarian or uterine cancer and the absence of the ovarian or uterine cancer based on at least abundance values for a fifth subset of the plurality of autoantibody species. The method thereby includes obtaining a probability or likelihood from the classifier that the subject has the ovarian or uterine cancer.

In some embodiments, the fifth subset of the plurality of autoantibody species includes at least 2 autoantibody species. Each respective autoantibody species of the at least 2 autoantibody species specifically binds to a different molecular target selected from those listed in Table 10.

In some embodiments, the method further includes, when the probability or likelihood obtained from the fifth classifier indicates that the subject has the ovarian or uterine cancer, administering a therapy for the ovarian or uterine cancer to the subject. The method also includes, when the probability or likelihood obtained from the classifier indicates that the subject does not have the ovarian or uterine cancer, forgoing administration of the therapy for the ovarian or uterine cancer to the subject.

In some embodiments, the method further includes, when the probability or likelihood obtained from the fifth classifier indicates that the subject has the ovarian or uterine cancer, confirming a diagnosis for ovarian or uterine cancer by further clinical evaluation. The confirming is performed prior to administering the therapy for the ovarian or uterine cancer to the subject.

Evaluating a Subject for a Disease State

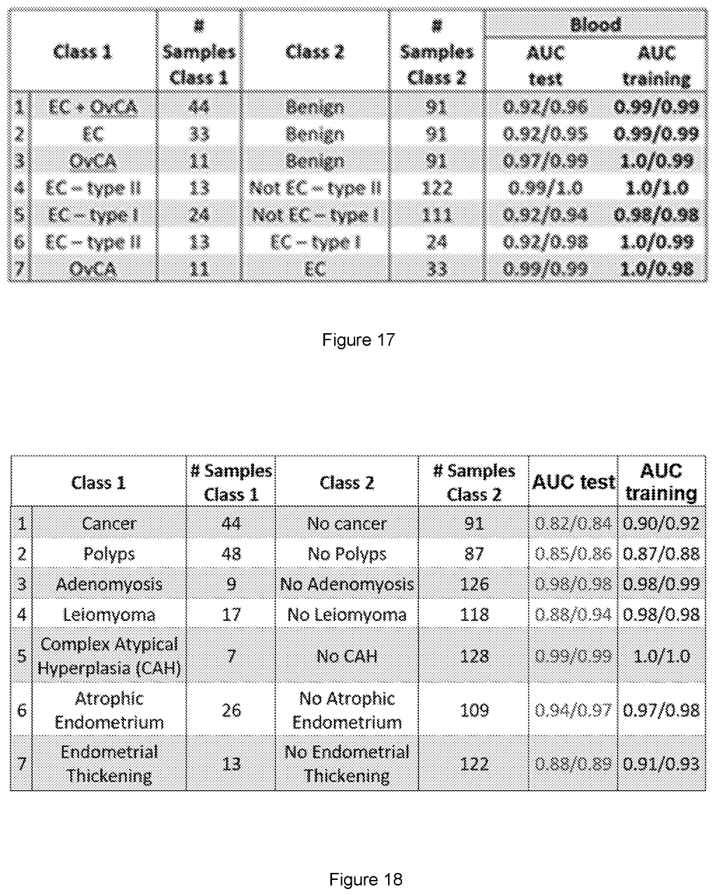

FIG. 16 illustrates example method 1600 evaluating a disorder in a subject using autoantibody biomarkers found in a biological sample, e.g., a liquid biological sample, from the subject.

In some embodiments, the disorder is an ovarian or uterine disease condition in a subject. In some embodiments, the ovarian or uterine disease condition is an ovarian cancer or an endometrial cancer. In some embodiments, the ovarian or uterine disease condition is adenomyosis, endometrial polyps, leiomyoma, or endometriosis (e.g., complex atypical hyperplasia and/or an atrophic endometrium and/or an endometrial thickening).

In some embodiments, the method evaluates a subject for a disease condition. In some such embodiments, the disease condition comprises a non-cancerous condition. In some embodiments, the non-cancerous condition is endometriosis, tuberculosis, fungal infections, or bacterial pneumonias. See Radha et al. et al. 2014 J Cytol. 31(3), 136-138. In some embodiments, the non-cancerous condition is pericoronitis, hematemesis, ulcerative colitis, ulcer, osteoarthritis, sinusitis, or other conditions known in the art.

In some such embodiments, the disease condition comprises a pre-cancerous or cancer condition. A pre-cancerous disease condition involves abnormal cells that are at an increased risk of developing into cancer. In some embodiments, the cancer condition comprises endometrial cancer, ovarian cancer, cervical cancer, uterine sarcoma, vaginal cancer, vulvar cancer, gestational trophoblastic disease, or other reproductive cancer. In some embodiments, the cancer condition comprises breast cancer, esophageal cancer, lung cancer, renal cancer, colorectal cancer, nasopharyngeal cancer, lymphoma, or any other cancer condition known in the art.

In some embodiments, the stage of endometrial cancer comprises stage 0 endometrial cancer (e.g., complex atypical hyperplasia), stage IA endometrial cancer, stage IB endometrial cancer, stage II endometrial cancer, stage III endometrial cancer, or stage IV endometrial cancer. In some embodiments, the stage of ovarian cancer comprises stage 0 ovarian cancer, stage IA ovarian cancer, stage IB ovarian cancer, stage II ovarian cancer, stage III ovarian cancer, or stage IV ovarian cancer.

In some embodiments, the subject is asymptomatic for endometrial cancer. In some embodiments, the subject is asymptomatic for ovarian and/or endometrial cancer. In some embodiments, subjects are asymptomatic for endometrial cancer but do exhibit complex atypical hyperplasia (CAH). This is a pre-cancerous state (e.g., equivalent to stage 0 endometrial cancer) that is associated with an approximately 40% increased risk of a subject developing endometrial cancer. See e.g., Suh-Burgmann et al. et al. 2009 Obstetrics and Gynecology 114(3), 523-529. In some embodiments, the subject is symptomatic for ovarian and/or endometrial cancer. In some embodiments, a subject is from a population with an increased risk for ovarian and/or endometrial cancer. In some embodiments, the increased risk is that the subject has Lynch syndrome, the subject is obese, the subject has family history of ovarian and/or endometrial cancer, the subject has a BRCA mutation, and/or the subject is over a predetermined age—e.g., where the predetermined age is at least 40, at least 45, at least 50, at least 55, at least 60, at least 65, or at least 70 years of age). In some embodiments, the subject is asymptomatic. In some embodiments, the subject is experiencing pelvic pain, abnormal bleeding, or infertility.

In some embodiments, a subject is concurrently evaluated for a stage of an additional cancer condition distinct from ovarian and endometrial cancer. In some embodiments, another cancer condition is selected from the group consisting of lung cancer, prostate cancer, colorectal cancer, renal cancer, cancer of the esophagus, cervical cancer, bladder cancer, gastric cancer, nasopharyngeal cancer, or a combination thereof.

Referring to block 1604, the evaluation method proceeds by obtaining a first biological sample, e.g., a biological fluid sample, from the subject. In some embodiments, the first biological fluid sample includes blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings. In some embodiments, the first biological fluid sample is a uterine lavage fluid. In some embodiments, a uterine lavage fluid is collected from the subject via hysteroscopy combined with curettage. In some embodiments, uterine lavage fluid is collected from the subject via uterine washings.

In some embodiments, a body cavity from which the lavage fluid sample is collected determines which type(s) of cancer said lavage fluid sample is assayed for (e.g., bladder cancer, oral cancer, lung cancer, gastrointestinal cancer, endometrial, and/or ovarian). In some such embodiments, the method further evaluates the subject for a stage of bladder cancer, a stage of oral cancer, a stage of lung cancer, a stage of gastrointestinal cancer, a stage of endometrial cancer, and/or a stage of ovarian cancer, respectively.

Referring to block 1606, the evaluation method proceeds by determining for each autoantibody species in a first set of autoantibody species, a corresponding abundance value for the respective autoantibody species in the first biological fluid sample. The method thereby includes obtaining an autoantibody abundance dataset for the subject. In some embodiments, the determining includes detectably binding each autoantibody to its cognate protein autoantigen. In some embodiments, the first set of autoantibody species was identified from training data for a larger plurality of autoantibody species using a feature extraction method.

In some embodiments, for each autoantibody species in the first set of autoantibody species, the corresponding abundance value for the respective autoantibody species includes an abundance of IgG and IgA homologues of the first set of autoantibody species in the biological fluid sample. In some embodiments, the IgG and IgA profiles are combined, thereby determining the respective abundance level of each autoantibody in the plurality of autoantibodies. In some embodiments, only one of either of the IgG or IgA profiles is used.

In some embodiments, the first set of autoantibody species includes at least 3 autoantibody species. In some embodiments, each respective autoantibody species of the at least 3 autoantibody species specifically binds to a different molecular target selected from those listed in Table 3. In some embodiments, the first set of autoantibody species includes at least 5 autoantibody species. In some embodiments, each respective autoantibody species of the at least 5 autoantibody species specifically binds to a different molecular target selected from those listed in Table 3. In some embodiments, the first set of autoantibody species includes at least 10 autoantibody species. In some embodiments, each respective autoantibody species of the at least 10 autoantibody species specifically binds to a different molecular target selected from those listed in Table 3. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 3.

In some embodiments, the first set of autoantibody species includes at least 3 autoantibody species. In some embodiments, each respective autoantibody species of the at least 3 autoantibody species specifically binds to a different molecular target selected from those listed in Table 3. In some embodiments, the first set of autoantibody species includes at least 5 autoantibody species. In some embodiments, each respective autoantibody species of the at least 5 autoantibody species specifically binds to a different molecular target selected from those listed in Table 3. In some embodiments, the first set of autoantibody species includes at least 10 autoantibody species. In some embodiments, each respective autoantibody species of the at least 10 autoantibody species specifically binds to a different molecular target selected from those listed in Table 3. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 3.

In some embodiments, the first set of autoantibody species includes at least 3 autoantibody species. In some embodiments, each respective autoantibody species of the at least 3 autoantibody species specifically binds to a different molecular target selected from those listed in Table 4. In some embodiments, the first set of autoantibody species includes at least 5 autoantibody species. In some embodiments, each respective autoantibody species of the at least 5 autoantibody species specifically binds to a different molecular target selected from those listed in Table 4. In some embodiments, the first set of autoantibody species includes at least 10 autoantibody species. In some embodiments, each respective autoantibody species of the at least 10 autoantibody species specifically binds to a different molecular target selected from those listed in Table 4. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 4.

In some embodiments, the first set of autoantibody species includes at least 3 autoantibody species. In some embodiments, each respective autoantibody species of the at least 3 autoantibody species specifically binds to a different molecular target selected from those listed in Table 5. In some embodiments, the first set of autoantibody species includes at least 5 autoantibody species. In some embodiments, each respective autoantibody species of the at least 5 autoantibody species specifically binds to a different molecular target selected from those listed in Table 5. In some embodiments, the first set of autoantibody species includes at least 10 autoantibody species. In some embodiments, each respective autoantibody species of the at least 10 autoantibody species specifically binds to a different molecular target selected from those listed in Table 5. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 5.

In some embodiments, the first set of autoantibody species includes at least 3 autoantibody species. In some embodiments, each respective autoantibody species of the at least 3 autoantibody species specifically binds to a different molecular target selected from those listed in Table 6. In some embodiments, the first set of autoantibody species includes at least 5 autoantibody species. In some embodiments, each respective autoantibody species of the at least 5 autoantibody species specifically binds to a different molecular target selected from those listed in Table 6. In some embodiments, the first set of autoantibody species includes at least 10 autoantibody species. In some embodiments, each respective autoantibody species of the at least 10 autoantibody species specifically binds to a different molecular target selected from those listed in Table 6. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 6.

In some embodiments, the first set of autoantibody species includes at least 3 autoantibody species. In some embodiments, each respective autoantibody species of the at least 3 autoantibody species specifically binds to a different molecular target selected from those listed in Table 7. In some embodiments, the first set of autoantibody species includes at least 5 autoantibody species. In some embodiments, each respective autoantibody species of the at least 5 autoantibody species specifically binds to a different molecular target selected from those listed in Table 7. In some embodiments, the first set of autoantibody species includes at least 10 autoantibody species. In some embodiments, each respective autoantibody species of the at least 10 autoantibody species specifically binds to a different molecular target selected from those listed in Table 7. In some embodiments, the first set of autoantibody species includes at least 2, 3, 4, 5, 6, 7, 8, 9, 10, 11, 12, 13, 14, 15, 16, 17, 18, 19, 20, 25, 30, 35, 40, 45, 50, or more autoantibody species that specifically bind to a different molecular target selected from those listed in Table 7.

In some embodiments, the plurality of autoantibody species includes at least 5 autoantibody species. Each respective autoantibody species of the at least 5 autoantibody species binds to a molecular target in a different pathway or cell type signature selected from those listed in Table 1.

Referring to block 1607, method 1600 includes using the autoantibody abundance dataset to determine values for each of a first set of autoantibody abundance features, thereby obtaining a first feature dataset for the subject. As described herein, in some embodiments, the autoantibody abundance features are abundance values for autoantibodies species, logs of the autoantibody abundance values, or a normalized abundance value thereof. For instance, in some embodiments, a normalization technique is applied to the autoantibody abundance values or logs thereof, such as scaling to a range, clipping, log scaling, or determining a z-score.

Referring to block 1608, the first feature dataset is then input into a classifier trained to distinguish between at least two states of the disease condition based on at least values for the first set of autoantibody abundance features, thereby obtaining a probability or likelihood from the classifier that the subject has a particular state of the disease condition. As described above, many types of classifiers can be used in conjunction with the methods described herein.

In some embodiments, the classifier determines a disease profile Vₛ for the subject comprising a weighted sum Wₛ of the respective autoantibody abundance features in the first feature dataset. Wₛ is calculated as:

Wₛ=Σᵢ₌₁^{m}(AᵢEᵢ),

where Eᵢ is a value of a respective autoantibody abundance feature i, in the first feature dataset m autoantibody abundance features, determined for the autoantibody abundance dataset, and Aᵢ is a weight for autoantibody abundance feature i.

In some embodiments, for each respective autoantibody abundance feature i in the first set of m autoantibody abundance features, the weight Aᵢ is calculated as:

Aᵢ˜Dᵢ⁻¹Σⱼ₌₁^{k}([Cᵢⱼ]⁻¹Zⱼ),

where Dᵢ is the standard deviation of the value of autoantibody abundance feature i in a training set of biological samples. The training set includes a first subset of biological samples from training subjects having a first state of the disorder, and a second subset of biological samples from training subjects having a second state of the disorder. [Cᵢⱼ [symbol] is a matrix of pairwise correlation between the values of autoantibody abundance features i and j in the first training set, such that [Cᵢⱼ]⁻¹ is the reciprocal matrix of pairwise correlation, where k=m−1, and Zⱼ is a z-score for the values of autoantibody abundance feature j in the first training set. Zⱼ is calculated as:

$${Z}_{j} = \frac{{{( {E}_{j} 〉}_{1} - ( {E}_{j} 〉}_{2}}{{D}_{j}} ,$$

where [symbol] Eⱼ [symbol] ₁ is the average value of autoantibody abundance feature j determined for the first subset of biological samples, [symbol] Eⱼ [symbol] ₂ is the average value of autoantibody abundance feature j determined for the second subset of biological fluid samples, and Dⱼ is the standard deviation of the values of autoantibody abundance feature j in the training set of biological fluid samples.

In some embodiments, the classifier was trained to distinguish between the at least two states of the disease condition based on at least abundance values for the first set of autoantibody species and one or more secondary features of the subject.

In some embodiments, the classifier includes a molecular signature algorithm, a neural network algorithm, a support vector machine algorithm, a decision tree algorithm, an unsupervised clustering model algorithm, a supervised clustering model algorithm, or a regression model.

In some embodiments, the disease condition is an ovarian cancer or an endometrial cancer. The one or more secondary features of the subject include two or more of the features selected from the group consisting of an age of the subject, a pregnancy history of the subject, a breastfeeding history of the subject, a BRCA1 genotype of the subject, a BRCA2 genotype of the subject, a breast cancer history of the subject, and a familial history of endometrial cancer, ovarian cancer, or breast cancer.

In some embodiments, the method further includes obtaining a second biological sample from the subject. In some embodiments, the second biological sample is a fluid sample. In some embodiments, the second biological sample includes blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings. In some embodiments, the fluid sample is a uterine lavage fluid or blood.

In some embodiments, the autoantibody abundance dataset for the subject further includes, for each autoantibody species in a second set of autoantibody species, a corresponding abundance value for the respective autoantibody species in the second biological sample.

In some embodiments, the method further includes obtaining nucleic acids from the first biological fluid sample or the second biological sample. The method includes sequencing with a predetermined minimum coverage value the nucleic acid sequences targeted by a panel of genes, thereby obtaining a set of gene expression levels for the subject. The method includes inputting the set of gene expression levels into the classifier. In some embodiments, the panel of genes includes at least 2 genes, at least 5 genes, at least 10 genes, at least 15 genes, or at least 20 genes.

### EXAMPLES

### Example 1: Proteomics Analysis of Lavage Fluid to Detect Early Stage Endometrial and Ovarian Cancers

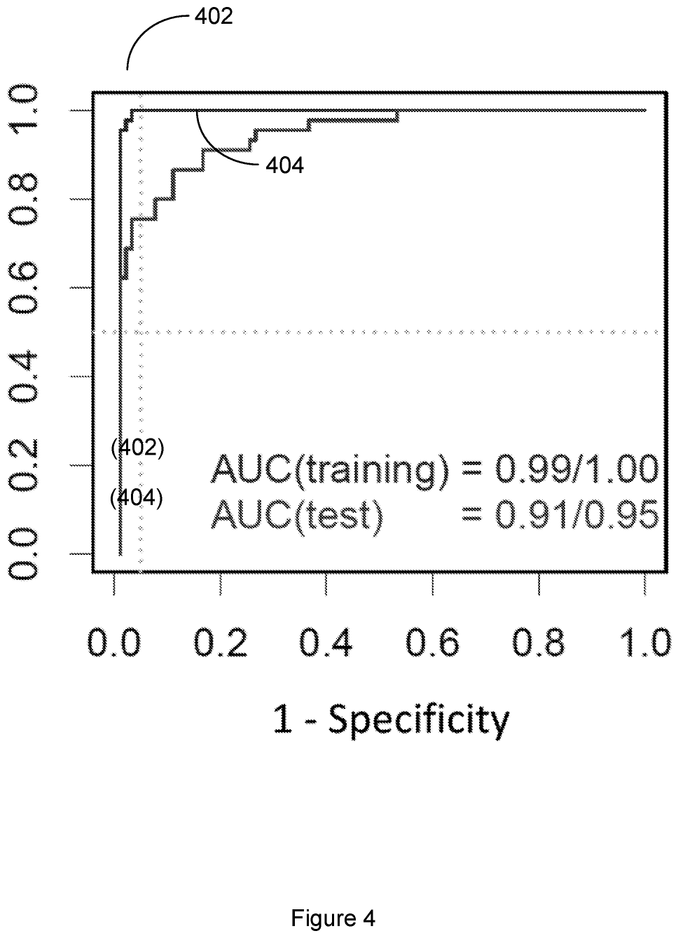

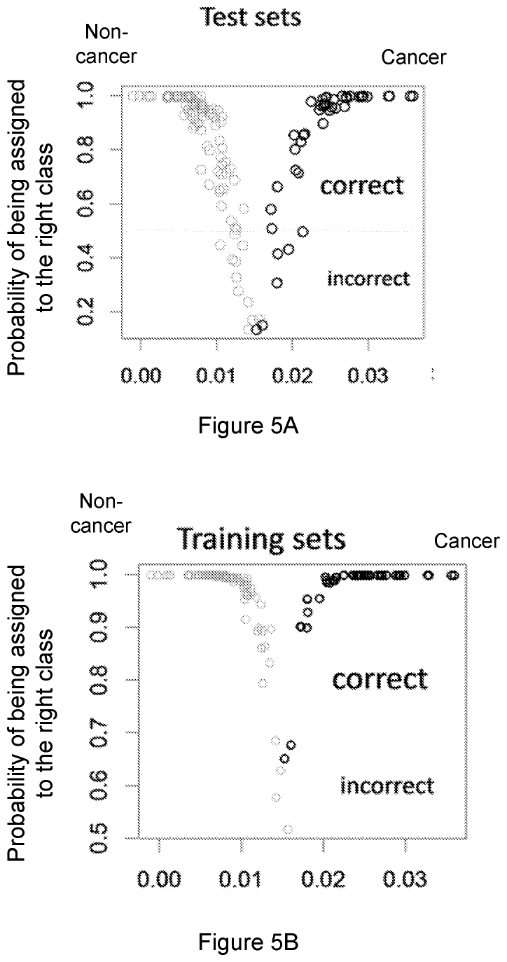

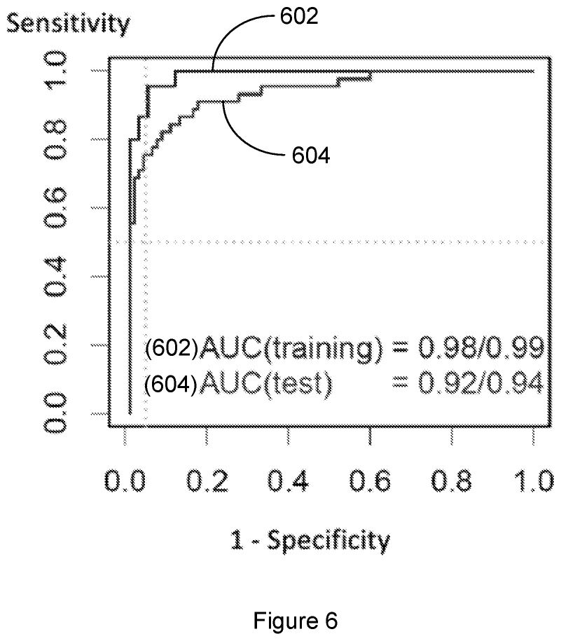

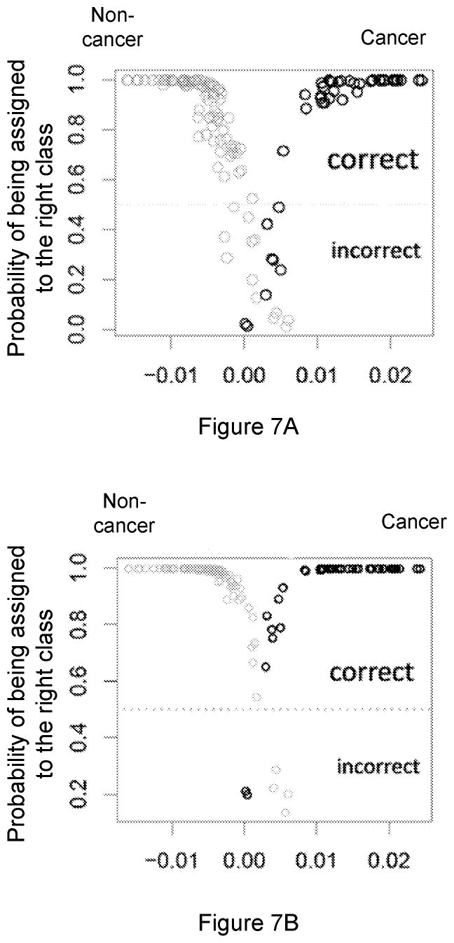

To determine the effectiveness of using a molecular signature to detect ovarian and endometrial cancer, at least 140 uterine lavage samples were collected from patients. Of these at least 140 samples, 30 samples were from patients with a stage of endometrial cancer, 10 samples were from patients with a stage of ovarian cancer, and at least 100 samples were from patients without cancer (e.g., were negative controls). Paired blood samples were also collected from each patient. The protein components of these uterine lavage samples were concentrated, and along with paired serum samples were analyzed using the HuProt™ Human Proteome Microarray from the Center for Diagnostic Imaging (CDI). See https://cdi-lab.com/HuProt.shtml. The resulting IgA and IgG profiles were then evaluated using a molecular signature model (MSM) classifier that was trained as described herein. The results of the MSM classifier are illustrated in FIGS. 4-7B. FIGS. 4, 5A, and 5B show that IgG and IgA profiles used in combination correctly classify the majority of samples. FIGS. 6, 7A, and 7B demonstrate that IgG profiles alone also can provide correct classifications.

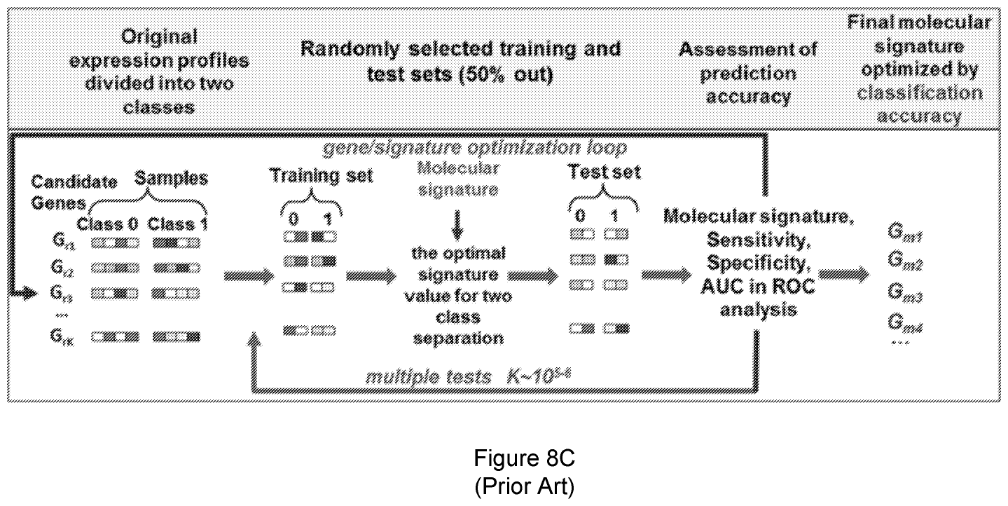

FIGS. 9A-9C further demonstrate that various gynecological diseases can, in some embodiments, be correctly classified using IgG and IgA profiles analyzed with the MSM classifier. These examples specifically represent the results of classifiers trained to output binary results (e.g., the patient has a respective clinical diagnosis or not). In FIGS. 9A, 9B, and 9C a classifier is trained using a plurality of reference subjects, where at least some of the reference subjects have a clinical diagnosis of endometrial polyps (e.g., the respective disease condition is endometrial polyps), and at least some of the reference subjects do not have a clinical diagnosis of endometrial polyps (e.g., control subjects who lack the respective disease condition).

### Example 2: Defining an Optimized Biomarker Panel

Our database of uterine lavage autoantibody profiles includes 935 patients (635 symptomatic individuals and 300 control individuals). The respective uterine lavage autoantibody profile for each patient is analyzed to obtain the complete autoantibody content (e.g., by using the HuProt™ Human Proteome Microarray from the Center for Diagnostic Imaging (CDI)). See https://cdi-lab.com/HuProt.shtml.

In some embodiments, an AAb biomarker panel is developed that can produce a high probability diagnostic risk score for each disease. To ensure a sample size that enables confident construction of the risk classification scoring system, AAb profiling of an additional training set of 800 biobanked, clinicopathologically annotated uterine lavage samples was performed. Once these 135 samples were analyzed to produce preliminary data, there were >150 samples for each of the four target diseases (e.g., “adenomyosis,” “endometrial polyps,” “leiomyoma,” and “endometriosis”) and two control sets (e.g., “no disease” and “other gynecologic diseases”). A machine learning model (e.g., as described above with regards to blocks 302-310) is then applied to this combined database of 935 profiles to construct classification scoring functions for distinguishing between the different disease states and controls (a total of 6 categories). This process includes: (i) assessing the statistical power of revealed AAb biomarkers, (ii) making specific false discovery rate corrections by generating synthetic datasets of the same 935 profiles and larger datasets, (iii) defining sensitivities and correlation structure of actual biomarkers compared to biomarkers derived from different synthetic sets, and (iv) developing the optimized single diagnostic panel of biomarkers for use in the commercial test by implementing entropy-based scoring of optimally selected subsets of AAb biomarkers. A prototype single diagnostic panel consisting of ˜200 AAbs was identified, where the diagnostic panel provides a specific risk score for each of the 4 conditions (adenomyosis, polyps, leiomyoma, and endometriosis). The AAbs are selected to ensure greater than 90% specificity for more than half these diseases.

### Example 3: Validating Optimized Biomarker Panel

The single diagnostic panel (e.g., the minimum AAbs set) developed in Example 2 was validated using a blinded preliminary validation and performance study to provide proof-of-concept for clinically useful sensitivity and specificity. An independent set of 300 uterine lavage samples were obtained and evenly divided between the different target diseases, adenomyosis, endometrial polyps, leiomyoma, endometriosis, and the two control populations as described in Example 2 (e.g., there are 50 reference subjects in each population). The single validated biomarker panel of ˜200 AAbs demonstrated greater than 90% specificity for at least 50% of each of these gynecologic diseases.

### Example 4: Development of an AAb Biomarker Panel that Produces a High Probability Risk Score for Each Cancer

To ensure a sample size that will facilitate construction of an actionable cancer risk classification scoring system, AAb profiling will be performed on an additional training set of 510 biobanked, clinicopathologically annotated blood samples including 175 women with Stage I cancers. Using these data and the improved ML-method described herein, a prototype diagnostic panel consisting of ˜200 AAbs, producing distinct classification scoring functions for distinguishing between the following diagnoses: cancer vs no cancer, EndoCA vs OvCA, and type I vs II EndoCA subtypes, will be identified. The following steps will be performed: (i) assess the statistical power of revealed AAb biomarkers, (ii) make false discovery rate corrections specific to our tasks by generating synthetic datasets of the same 635 profiles and larger datasets, (iii) study sensitivities and correlation structure of actual biomarkers compared to those derived from different synthetic sets, and (iv) develop the optimal diagnostic panel of biomarkers for use in the commercial test by implementing entropy-based scoring of optimally selected subsets of AAb biomarkers. A prototype diagnostic panel consisting of ˜200 AAbs that will produce distinct classification scoring functions for distinguishing between all groups, with ≥80% overall accuracy for all classifications.

### Example 5: Proof-of-Concept Validation Study and Panel Refinement

Using the prototype panel described in Example 4, a blinded preliminary validation, performance, and optimization study will be performed using an independent set of 210 biobanked blood samples to demonstrate proof-of-concept for clinically useful sensitivity and specificity and further minimize and finalize the panel to ˜100 AAbs. Samples will be one third OvCA (Stages I to IV), one third EndoCA (type I and II, Stages I to IV), and one third benign controls. Milestone. A final, optimized biomarker panel of ˜100 AAbs that will provide ≥90% specificity for ≥50% of cancer vs. no cancer samples, and ≥80% specificity for ≥50% of the remaining diagnostic groups (with ≥50% of Stage 1 cancers demonstrating ≥80% specificity).

At the end of Phase I, an optimized single panel of ˜100 AAbs will be identified that meet selected performance metrics and position MDDx for commercial test development and a prospective clinical validation study in Phase II directed towards FDA regulatory approval. Given lethality and quality-of-life differences between early- and late-stage OvCA, and the distinct survival, treatment and management options for type I and II EndoCA, this single molecular panel will provide actionable information to guide patient management. MDDx's screening test will reduce health care costs associated with late-stage cancer surgery and care, improve racial/ethnic disparities in diagnosis and outcome, and improve overall survival and quality of life for women with these cancers.

### Example 6: Proof of Concept

The approach described herein is distinct. The approach starts by having access to a rich source of matched blood samples all with linked clinical information from patients enrolled by our multi-institutional registry. The preliminary discovery analysis described herein is based on a cohort of 135 women (10 OvCA (all serous histology; stages I-IV), 35 EndoCA (types I and II, stages I-IV), 90 benign controls) and plan to include an additional 510 women (evenly split between OvCA, EndoCA, and benign controls, with 175 Stage I cancer samples) for a total discovery cohort of 645 women. This will be the largest discovery cohort ever used, using the complete proteome as the AAb discovery template, and analysis will be performed by a novel and powerful ML method that has been able to identify diagnostic AAbs with high confidence, most notably even in Stage I disease. All samples from women with and without cancer are/will be obtained from women presenting for hysteroscopy with dilation and curettage (D&C) for diagnostic evaluation due to abnormal bleeding, pelvic pain, or abnormal results following sonohysterogram.

This discovery cohort represents a distinct population of women seeking medical care, thus validation set (SA #2)—an independent set of 210 control samples from women with and without cancer and notably, controls who are women without gynecologic complaints but who provided blood samples during their routine annual gynecologic visit—provides a powerful control set for true population studies. Given the different prevalence of OvCA and EndoCA, test sensitivities and specificities will need to be defined during Phase II studies for clinically relevant results. This approach is distinct from previously published efforts and methods under clinical trial, and it will produce a diagnostic panel that will be powerful enough to employ as a screening test for OvCA and EndoCA.

This novel biomarker screening test could be applied to the ˜82+ million U.S. women over the age of 40 at the time of an office visit as part of an annual screening tool. This will be a low-cost screening array that contains antigens for the full set of diagnostic AAbs and a number of controls along with an analysis program that will return classification results to be communicated to the provider with actionable directives for the patient. The format of the final array is still to be determined; however, it will likely be a modification of the CDI array or a bead-based multiplex Luminex-style array, for testing to be performed by commercial testing laboratories. Currently, samples for these studies were drawn from women undergoing invasive laparoscopy or hysteroscopy with dilation and curettage (D&C) for diagnostic evaluation. Our assay will replace this OR-based method of diagnosis (costing an average of >$14,600 per procedure) and enable office-based screening of asymptomatic women. With the shift toward value-based care models, screening to detect expensive and potentially fatal diseases at an early stage when simpler and more cost-effective treatment options are available will be essential to drive down costs while maximizing value.

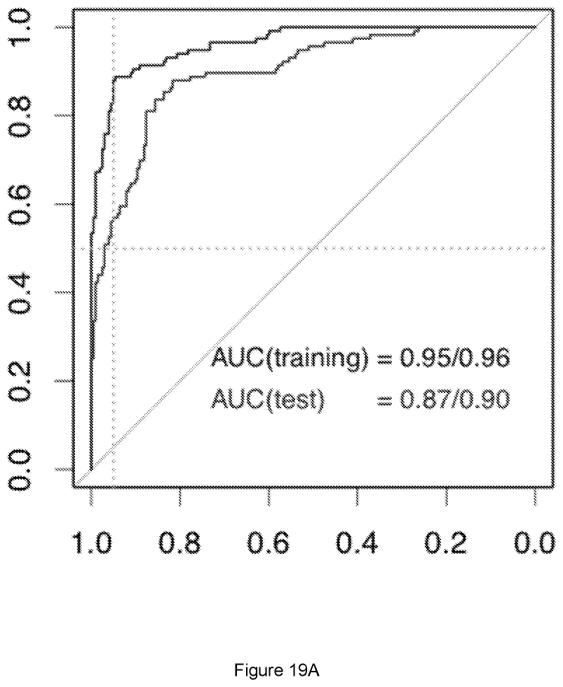

Briefly, the complete autoantibody content of plasma samples obtained from 135 women were analyzed using CDI's HuProt Array and demonstrated that an AAb classification signature of 24 biomarkers can be used to differentiate between women with and without cancer with accuracies of ˜90% or higher. Essentially, biomarkers (AAbs) that are differentially expressed between two groups, for instance cancer vs. benign, are identified. Then, subsets of AAbs are sampled to rank biomarkers and to create biomarker signatures capable to classify a given group of samples. The ML algorithm consecutively tests all signatures (2, 3, “ . . . ”, N biomarkers) and determines the one with the highest predictive accuracy. Notably, (1) nearly all stage I cancers were correctly detected (3/3 OvCA and 23/24 EndoCA), (2) OvCA was well distinguished from EndoCA, and (3) different EndoCA subtypes (endometriod, type I and serous, type II) were distinguishable with high specificity and sensitivity (FIG. 17). Currently, each patient is screened using the entire 21,000 protein HuProt array. The goal of this Phase I proposal is to refine this current platform into an affordable, easy-to-use, high confidence and pre-defined single panel of 100 biomarkers or less.

As shown in FIG. 17, there are a total of 7 diagnostic classifications that need to be computationally assessed as part of our goal of defining one final clinically useful panel (e.g., 3 diagnostic categories). To do so, we will obtain IgA and IgG AAb profiles of an additional training set of 510 biobanked, clinic-pathologically annotated blood samples from women with and without cancer. By adding these new samples to the original 135 patients (preliminary data) we will have sufficient power to confidently apply our newly developed ML approaches to construct a cancer classification scoring function for distinguishing between (1) cancer and no cancer, (2) OvCA vs. EndoCA, and (3) type I vs II EndoCA, regardless of cancer stage. We will use our ML computational protocol that we have successfully applied to classify our preliminary data set of 135 patients to analyze the expanded dataset. Analysis will include (i) assessment of statistical power of revealed AAb biomarkers, (ii) false discovery rate corrections specific to our tasks through generation of synthetic datasets (n=645 and larger), and (iii) defining sensitivities and correlation structure of actual biomarkers compared to biomarkers derived from different synthetic sets. Using the results from this analysis we will then develop a minimal diagnostic panel of biomarkers and implement entropy-based scoring of optimally selected subsets of AAb biomarkers. Importantly, the scoring is not a simple binary present or absent, but is a measure of the relative expression level of each AAb. This type of scoring based on relative expression levels will reduce batch effects while preserving classification accuracy. OvCA samples (n=170; Stages I-IV, Stage I n=15) will be high grade serous ovarian cancer. EndoCA will include 120 type I endometrioid and 50 type II serous histologies, Stages I-IV (Stage I, n=110).

This classification approach is based on the optimal combination of statistically significant and independent (correlation <1) biomarkers with relatively low sensitivity. With this approach, the overall classification accuracy will depend on how well the sensitivities of biomarkers derived from a particular training database reproduce its true population sensitivity. Our estimates demonstrated that analysis of ˜200 samples for each subtype (OvCA, EndoCA, and benign) will make it possible to reliably determine biomarkers of population sensitivity ˜60% at a probability of ˜5% fluctuation less than 0.01 (sensitivity of 50%=random association). In practice, diagnostic power depends on the actual population distribution of biomarkers by sensitivity. This can be illustrated by the following example: a classification function of 5 biomarkers of sensitivity ˜₇₀% can classify only 25% of samples with specificity of 0.95; by adding 10 more biomarkers of sensitivity 60%, ˜50% of samples will be classified with specificity of 0.95; adding 15 more biomarkers of sensitivity 55% will make it possible to classify ˜₈₀% of samples with a specificity of 0.95, and so on.

The blood samples used for this example were collected and biobanked from consenting patients who underwent hysteroscopy and curettage for diagnostic evaluation of abnormal uterine bleeding or abnormal pelvic ultrasound under existing IRBs (GCO #10-1166 (Sinai) and BRANY 13-02-356-337(Danbury)). Following collection, plasma was isolated and aliquoted into at least five vials of 200 μL each frozen at −80° C. within 4 hours of blood draw. All 510 samples are available for profiling for this aim, and approximately 50 additional samples are collected on average each month should the need for additional samples arise. Based on current biobank statistics, it is expected that women of all races and ethnicities will continue to be represented in these studies and roughly reflect the demographics of our catchment areas and communities.

CDI Laboratories' HuProt Microarray contains >21,000 GST-purified recombinant, full-length proteins (covering 16,794 unique genes, >81% of the canonical human proteome) that were expressed in yeast to ensure correct folding and eukaryotic post-translational modifications. Innovative and unique aspects of the platform have been described above. For each patient and analysis, 200 μL of plasma (˜20 μl/run), stripped of any identifying labels other than laboratory assigned coding numbers, will be profiled by CDI who in turn will provide RAW readouts to MDDx for ML analysis.

CDI has demonstrated robust reproducibility of HuProt microarray data between individual slides. Serum collected from a healthy adult human male donor was incubated on pairs of HuProt proteome microarrays across three print batches (Batch 1; Feb12_2020, Batch 2; Dec09_2019, Batch 3; Oct01_2010), and stained with anti-IgG and anti-IgA secondaries. Raw data were plotted on a log scale and linear regression analysis was performed. Intra-lot correlations of spot pair averages (Rep 1 vs Rep 2 intra-lot) was >0.95 R2 within all three batches in both channels. Slide-to slide cross pairings across all possible pairs of the six slides was >0.90 R2 correlation. These results demonstrate that multi-sample, -batch, or -isotype analysis requiring multiple slides should be reliable.

### Example 7: Proof of Concept

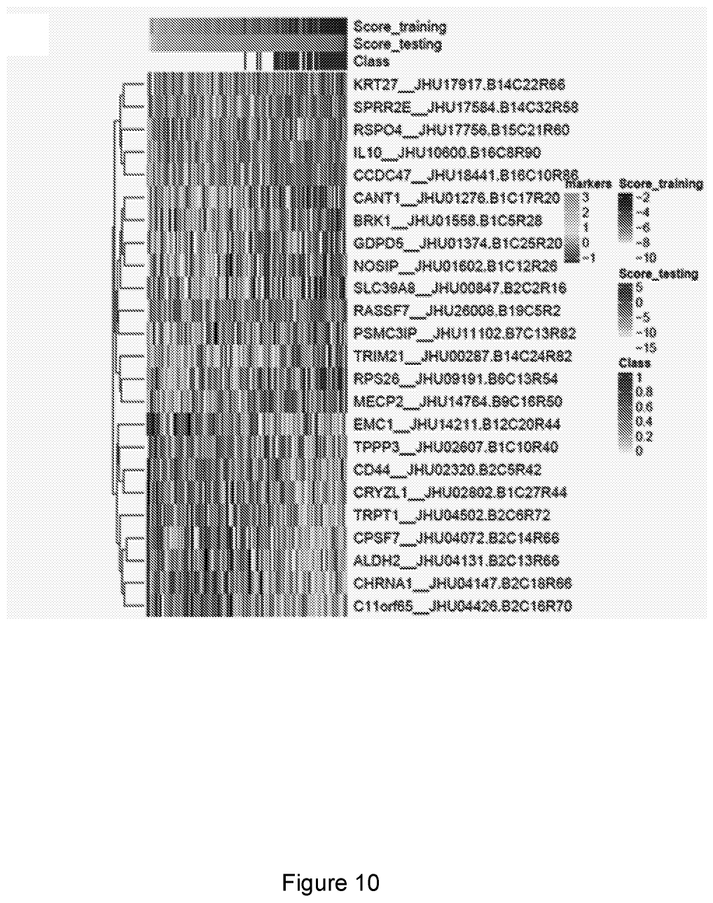

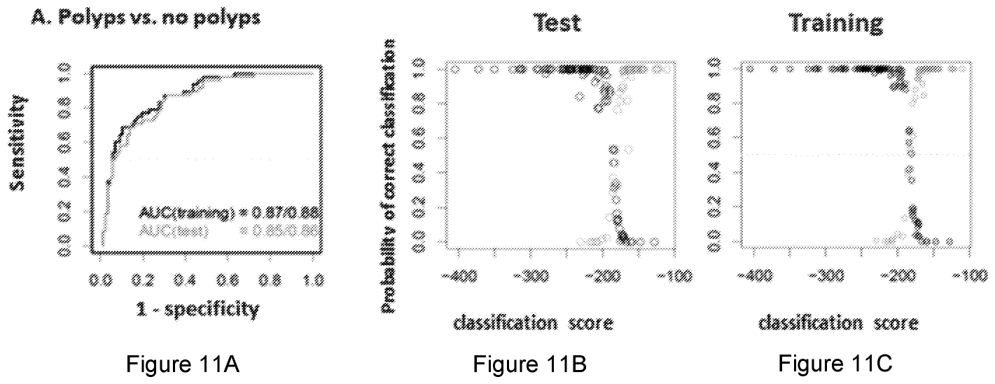

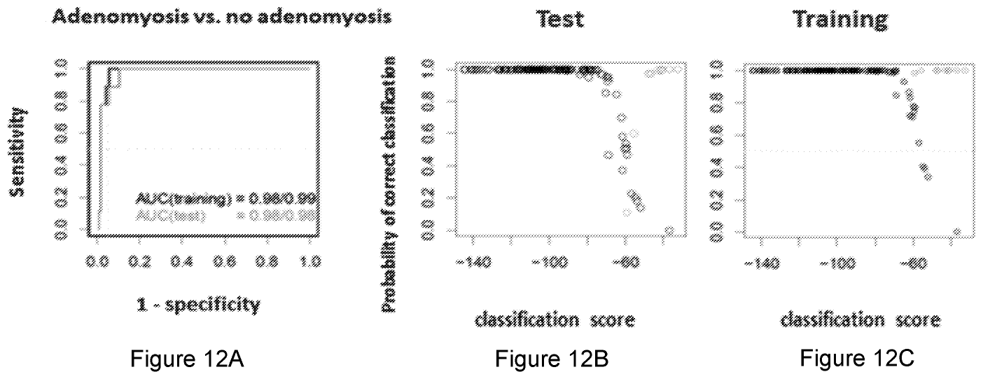

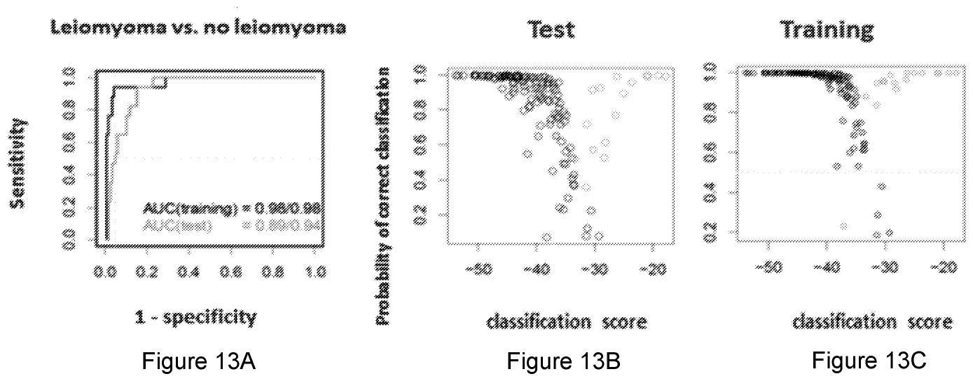

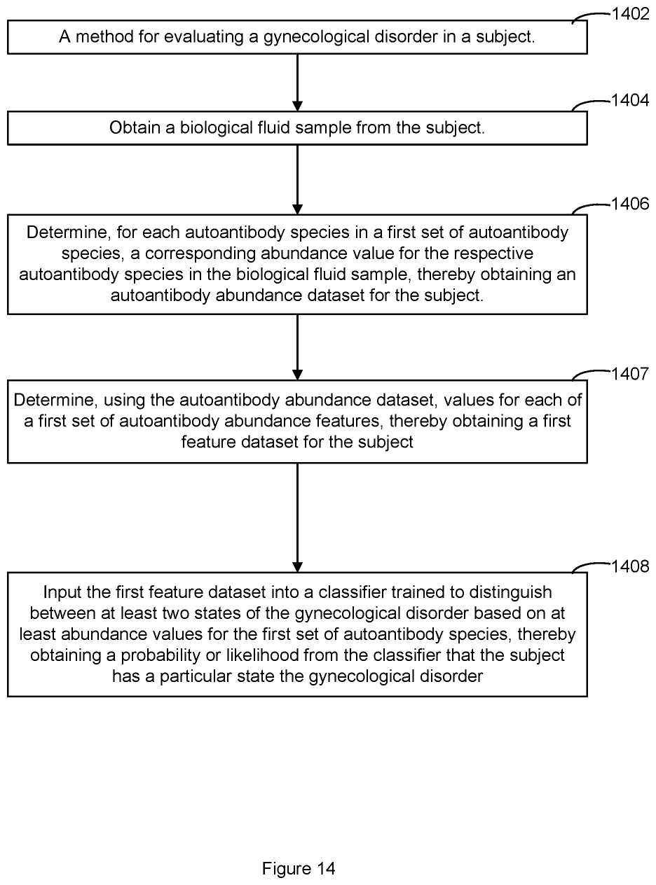

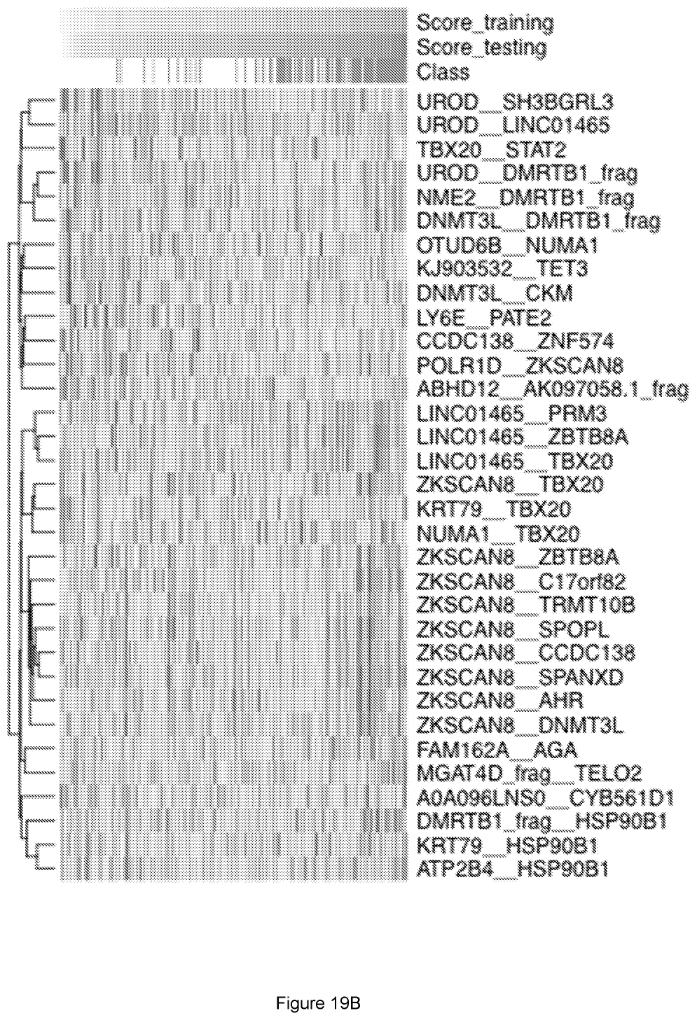

The complete autoantibody content of uterine lavage samples obtained from 135 women was analyzed using CDI's HuProt Human Proteome Array in combination with our ML tool and demonstrated that an AAb classification signature of <100 biomarkers can differentiate between women with and without a number of clinically-relevant gynecologic states with accuracies of ˜90% or higher (FIGS. 11-13 and 18). Taken together, these data demonstrate that these gynecologic diseases, including endometrial polyps, atrophic endometrium, leiomyomas, adenomyosis, endometrial thickening, complex atypical endometrial hyperplasia (a pre-neoplastic condition) and ovarian and endometrial cancers are associated with systematic changes in the content of immune proteins and that these molecular changes can be detected by AAb profiling and ML. While we have not yet defined the AAb profiles of our endometriosis patients, we have no reason to suspect that this will not be accomplished while this application is under review. These samples were unfortunately sent for analysis as the laboratories were being shut down in NYC secondary to the current health crisis.

Uterine lavage samples used for this example are continuously collected and biobanked from consenting patients who are undergoing hysteroscopy and D&C for diagnostic evaluation of pelvic pain and abnormal uterine bleeding, SIS for infertility evaluation, women undergoing ovarian and endometrial cancer surgery and women without evidence of disease who presented for routine gynecologic care and agreed to participate as controls, under existing IRBs (GCO #10-1166 (Sinai) and BRANY 13-02-356-337(Danbury)). For all, ˜20 ml of uterine lavage fluid is collected and biobanked. Given the location and catchment areas of our enrolling sites, and based on current biobank statistics, it is expected that women of all races and ethnicities will continue to be represented in these studies and roughly reflect the demographics of our catchment areas and communities.

### CONCLUSION

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s) described herein. In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed:

1. A method for evaluating a gynecological disorder in a subject, the method comprising:
a) obtaining a biological fluid sample from the subject;
b) determining, for each autoantibody species in a first set of autoantibody species, a corresponding abundance value for the respective autoantibody species in the biological fluid sample, thereby obtaining an autoantibody abundance dataset for the subject;
c) determining, using the autoantibody abundance dataset, values for each of a first set of autoantibody abundance features, thereby obtaining a first feature dataset for the subject; and
d) inputting the first feature dataset into a classifier trained to distinguish between at least two states of the gynecological disorder based on at least values for the first set of autoantibody abundance features, thereby obtaining a probability or likelihood from the classifier that the subject has a particular state of the gynecological disorder.

2. The method of claim 1, wherein the biological fluid sample is a blood sample or fraction thereof.

3. The method of claim 1, wherein the biological fluid sample is a uterine lavage sample.

4. The method of any one of claims 1-3, wherein the first set of autoantibody species comprises at least 5 autoantibody species, wherein each respective autoantibody species of the at least 5 autoantibody species specifically binds to a different molecular target selected from those listed in any of Tables 2-7.

5. The method of any one of claims 1-3, wherein the first set of autoantibody abundance features comprises at least 5 autoantibody abundance features, wherein each respective autoantibody abundance features of the at least 5 autoantibody abundance features is a comparison of the abundances of a pair of autoantibodies that specifically bind to a different pair of molecular targets selected from the pairs of molecular targets listed in any of Tables 2-7.

6. The method of any one of claims 1-5, wherein the first set of autoantibody species comprises at least 5 autoantibody species, wherein each respective autoantibody species of the at least 5 autoantibody species binds to a molecular target in a different pathway or cell type signature selected from those listed in Table 1.

7. The method of any one of claims 1-6, wherein each respective feature in the first set of autoantibody abundance features comprises a normalized abundance value for a respective autoantibody species in the first set of autoantibody species.

8. The method of any one of claims 1-6, wherein each respective feature in the first set of autoantibody abundance features comprises a comparison between an abundance value for a first respective autoantibody species in the first set of autoantibody species and an abundance value for a second respective autoantibody species in the first set of autoantibody species.

9. The method of any one of claims 1-8, wherein for each autoantibody species in the first set of autoantibody species, the corresponding abundance value for the respective autoantibody species comprises an abundance of IgG and IgA homologues of first set of autoantibody species in the biological fluid sample.

10. The method of any one of claims 1-9, wherein the classifier determines a disease profile Vₛ for the subject comprising a weighted sum Wₛ of the respective autoantibody abundance features in the first feature dataset, calculated as:
Wₛ=Σᵢ₌₁^{m}(AᵢEᵢ),
where:
Eᵢ is a value of a respective autoantibody abundance feature i, in the first feature dataset m autoantibody abundance features, determined for the autoantibody abundance dataset, and
Aᵢ is a weight for autoantibody abundance feature i.

11. The method of claim 10, wherein, for each respective autoantibody abundance feature i in the first set of m autoantibody abundance features, the weight Aᵢ is calculated as:
Aᵢ˜Dᵢ⁻¹Σⱼ₌₁^{k}([Cᵢⱼ]⁻¹Zⱼ),
where:
Dᵢ is the standard deviation of the value of autoantibody abundance feature i in a training set of biological fluid samples, wherein the training set comprises:
a first subset of biological fluid samples from training subjects having a first state of the gynecological disorder, and
a second subset of biological fluid samples from training subjects having a second state of the gynecological disorder;
Cᵢⱼ, is a matrix of pairwise correlation between the values of autoantibody abundance features i and j in the first training set, such that [Cᵢⱼ]⁻¹ is the reciprocal matrix of pairwise correlation, wherein k=m−1, and
Zⱼ is a z-score for the values of autoantibody abundance feature j in the first training set, calculated as:
$${Z}_{j} = \frac{{{( {E}_{j} 〉}_{1} - ( {E}_{j} 〉}_{2}}{{D}_{j}} ,$$
where: [symbol]
Eⱼ [symbol] ₁ is the average value of autoantibody abundance feature j determined for the first subset of biological fluid samples, [symbol]
Eⱼ [symbol] ₂ is the average value of autoantibody abundance feature j determined for the second subset of biological fluid samples, and
Dⱼ is the standard deviation of the values of autoantibody abundance feature j in the training set of biological fluid samples.

12. The method of any one of claims 1-11, wherein the classifier was trained to distinguish between the at least two states of the gynecological disorder based on at least the values for each of the first set of autoantibody abundance features and one or more secondary features of the subject.

13. The method of claim 12, wherein:
the gynecological disorder is an ovarian cancer or an endometrial cancer, and
the one or more secondary features of the subject comprise two or more of the features selected from the group consisting of an age of the subject, a body mass index of the subject, a pregnancy history of the subject, a breastfeeding history of the subject, a BRCA1 genotype of the subject, a BRCA2 genotype of the subject, a breast cancer history of the subject, and a familial history of endometrial cancer, ovarian cancer, or breast cancer.

14. The method of any one of claims 1-13, the method further comprising:
obtaining a second biological sample from the subject;
determining a plurality of secondary features from the second biological sample, thereby obtaining a secondary feature dataset for the subject; and
inputting the secondary feature dataset into the classifier.

15. The method of claim 14, wherein the second biological sample is a uterine lavage fluid.

16. The method of claim 14, wherein the second biological sample is a blood sample or a fraction thereof.

17. The method of any one of claims 1-16, wherein the gynecological disorder is an ovarian cancer or an endometrial cancer.

18. The method of claim 17, wherein the classifier was trained to distinguish between (i) the presence of an ovarian cancer or uterine cancer and (ii) the absence of the ovarian cancer or the uterine cancer, the method further comprising:
when the probability or likelihood obtained from the classifier indicates that the subject has the ovarian cancer or the uterine cancer, administering a therapy for the ovarian cancer or the uterine cancer to the subject, and
when the probability or likelihood obtained from the classifier indicates that the subject does not have the ovarian cancer or the uterine cancer, forgoing administration of the therapy for the ovarian cancer or the uterine cancer to the subject.

19. The method of claim 17, wherein the classifier was trained to distinguish between (i) a first stage of an ovarian cancer or uterine cancer and (ii) a second stage of the ovarian cancer or the uterine cancer that is more advanced than the first stage of the ovarian cancer or the uterine cancer, the method further comprising:
when the probability or likelihood obtained from the classifier indicates that the subject has the first stage of the ovarian cancer or the uterine cancer, administering a first therapy for the ovarian cancer or the uterine cancer to the subject, and
when the probability or likelihood obtained from the classifier indicates that the subject has the first stage of the ovarian cancer or the uterine cancer, administering a second therapy for the ovarian cancer or the uterine cancer to the subject.

20. The method of any one of claims 1-16, wherein the gynecological disorder is adenomyosis, endometrial polyps, leiomyoma, or endometriosis.

21. The method of claim 20, wherein the classifier was trained to distinguish between (i) the presence of adenomyosis, endometrial polyps, leiomyoma, or endometriosis and (ii) the absence of the adenomyosis, endometrial polyps, leiomyoma, or endometriosis, the method further comprising:
when the probability or likelihood obtained from the classifier indicates that the subject has the adenomyosis, endometrial polyps, leiomyoma, or endometriosis, administering a therapy for the adenomyosis, endometrial polyps, leiomyoma, or endometriosis to the subject, and
when the probability or likelihood obtained from the classifier indicates that the subject does not have the adenomyosis, endometrial polyps, leiomyoma, or endometriosis, forgoing administration of the therapy for the adenomyosis, endometrial polyps, leiomyoma, or endometriosis to the subject.

22. The method of any one of claims 1-16, wherein the gynecological disorder is infertility.

23. The method of any one of claims 1-22, wherein the subject is asymptomatic.

24. The method of any one of claims 1-22, wherein the subject is experiencing pelvic pain, abnormal bleeding, or infertility.

25. The method of any one of claims 1-22, wherein the subject is perimenopausal or post-menopausal.

26. The method of any one of claims 1-22, wherein the subject has a family history of gynecologic cancer or gynecologic disease.

27. A method for evaluating a gynecological disorder in a subject, the method comprising:
a) obtaining a biological fluid sample from the subject;
b) determining, for each autoantibody species in a plurality of autoantibody species, a corresponding abundance value for the respective autoantibody species in the biological fluid sample, thereby obtaining a master autoantibody abundance dataset for the subject;
c) inputting a first subset of the master autoantibody abundance dataset into a first classifier trained to distinguish between the presence of adenomyosis and the absence of adenomyosis based on at least abundance values for a first subset of the plurality of autoantibody species, thereby obtaining a probability or likelihood from the classifier that the subject has adenomyosis;
d) inputting a second subset of the master autoantibody abundance dataset into a second classifier trained to distinguish between the presence of endometrial polyps and the absence of endometrial polyps based on at least abundance values for a second subset of the plurality of autoantibody species, thereby obtaining a probability or likelihood from the classifier that the subject has endometrial polyps;
e) inputting a third subset of the master autoantibody abundance dataset into a third classifier trained to distinguish between the presence of leiomyoma and the absence of leiomyoma based on at least abundance values for a third subset of the plurality of autoantibody species, thereby obtaining a probability or likelihood from the classifier that the subject has leiomyoma; and
f) inputting a fourth subset of the master autoantibody abundance dataset into a fourth classifier trained to distinguish between the presence of endometriosis and the absence of endometriosis based on at least abundance values for a fourth subset of the plurality of autoantibody species, thereby obtaining a probability or likelihood from the classifier that the subject has endometriosis.

28. The method of claim 27, wherein the biological fluid sample is a blood sample or fraction thereof.

29. The method of claim 27, wherein the biological fluid sample is a uterine lavage sample.

30. The method of any one of claims 27-29, wherein the plurality of autoantibody species comprises at least 5 autoantibody species, wherein each respective autoantibody species of the at least 5 autoantibody species specifically binds to a different molecular target selected from those listed in any of Tables 2-15.

31. The method of any one of claims 27-30, wherein the plurality of autoantibody species comprises at least 5 autoantibody species, wherein each respective autoantibody species of the at least 5 autoantibody species binds to a molecular target in a different pathway or cell type signature selected from those listed in Table 1.

32. The method of any one of claims 27-32, further comprising:
when the probability or likelihood obtained from the first classifier indicates that the subject has adenomyosis, administering a therapy for adenomyosis to the subject,
when the probability or likelihood obtained from the second classifier indicates that the subject has endometrial polyps, administering a therapy for endometrial polyps to the subject,
when the probability or likelihood obtained from the third classifier indicates that the subject has leiomyoma, administering a therapy for leiomyoma to the subject,
when the probability or likelihood obtained from the fourth classifier indicates that the subject has endometriosis, administering a therapy for endometriosis to the subject, and
when the probabilities or likelihoods obtained from the first through fourth classifiers indicates that the subject does not have at least one condition selected from the group consisting of adenomyosis, endometrial polyps, leiomyoma, and endometriosis, forgoing administration of the therapies for adenomyosis, endometrial polyps, leiomyoma, and endometriosis.

33. The method of claim 32, further comprising, when the probabilities or likelihoods obtained from the first through fourth classifiers indicates that the subject has at least one condition selected from the group consisting of adenomyosis, endometrial polyps, leiomyoma, and endometriosis:
confirming a diagnosis for the at least one condition selected from the group consisting of adenomyosis, endometrial polyps, leiomyoma, and endometriosis by further clinical evaluation, prior to administering the therapy for the at least one condition selected from the group consisting of adenomyosis, endometrial polyps, leiomyoma, and endometriosis to the subject.

34. The method of any one of claims 27-33, further comprising:
g) inputting a fifth subset of the master autoantibody abundance dataset into a fifth classifier trained to distinguish between the presence of an ovarian or uterine cancer and the absence of the ovarian or uterine cancer based on at least abundance values for a fifth subset of the plurality of autoantibody species, thereby obtaining a probability or likelihood from the classifier that the subject has the ovarian or uterine cancer.

35. The method of claim 34, further comprising:
when the probability or likelihood obtained from the fifth classifier indicates that the subject has the ovarian or uterine cancer, administering a therapy for the ovarian or uterine cancer to the subject, and
when the probability or likelihood obtained from the classifier indicates that the subject does not have the ovarian or uterine cancer, forgoing administration of the therapy for the ovarian or uterine cancer to the subject.

36. The method of claim 35, further comprising, when the probability or likelihood obtained from the fifth classifier indicates that the subject has the ovarian or uterine cancer:
confirming a diagnosis for ovarian or uterine cancer by further clinical evaluation, prior to administering the therapy for the ovarian or uterine cancer to the subject.

37. The method of any one of claims 27-36, wherein for each autoantibody species in the plurality of autoantibody species, the corresponding abundance value for the respective autoantibody species comprises an abundance of IgG and IgA homologues of the plurality of autoantibody species in the biological fluid sample.

38. The method of any one of claims 27-37, wherein the subject is asymptomatic.

39. The method of any one of claims 27-37, wherein the subject is experiencing pelvic pain, abnormal bleeding, or infertility.

40. A method for evaluating a disease condition in a subject, the method comprising:
a) obtaining a first biological fluid sample from the subject;
b) determining, for each autoantibody species in a first set of autoantibody species, a corresponding abundance value for the respective autoantibody species in the first biological fluid sample, thereby obtaining an autoantibody abundance dataset for the subject;
c) determining, using the autoantibody abundance dataset, values for each of a first set of autoantibody abundance features, thereby obtaining a first feature dataset for the subject; and
d) inputting the first feature dataset into a classifier trained to distinguish between at least two states of the disease condition based on at least values for the first set of autoantibody abundance features, thereby obtaining a probability or likelihood from the classifier that the subject has a particular state of the disease condition.

41. The method of claim 40, wherein the classifier determines a disease profile Vₛ for the subject comprising a weighted sum Wₛ of the respective autoantibody abundance features in the first feature dataset, calculated as:
Wₛ=Σᵢ₌₁^{m}(AᵢEᵢ),
where:
Eᵢ is a value of a respective autoantibody abundance feature i, in the first feature dataset m autoantibody abundance features, determined for the autoantibody abundance dataset, and
Aᵢ is a weight for autoantibody abundance feature i.

42. The method of claim 41, wherein, for each respective autoantibody abundance feature i in the first set of m autoantibody abundance features, the weight Aᵢ is calculated as:
Aᵢ˜Dᵢ⁻¹Σⱼ₌₁^{k}([Cᵢⱼ]⁻¹Zⱼ),
where:
Dᵢ is the standard deviation of the value of autoantibody abundance feature i in a training set of uterine lavage fluid samples, wherein the training set comprises:
a first subset of uterine lavage fluid samples from training subjects having a first state of the gynecological disorder, and
a second subset of uterine lavage fluid samples from training subjects having a second state of the gynecological disorder;
Cᵢⱼ, is a matrix of pairwise correlation between the values of autoantibody abundance features i and j in the first training set, such that [Cᵢⱼ]⁻¹ is the reciprocal matrix of pairwise correlation, wherein k=m−1, and
Zⱼ is a z-score for the values of autoantibody abundance feature j in the first training set, calculated as:
$${Z}_{j} = \frac{{{( {E}_{j} 〉}_{1} - ( {E}_{j} 〉}_{2}}{{D}_{j}} ,$$
where: [symbol]
Eⱼ [symbol] ₁ is the average value of autoantibody abundance feature j determined for the first subset of uterine lavage fluid samples, [symbol]
Eⱼ [symbol] ₂ is the average value of autoantibody abundance feature j determined for the second subset of uterine lavage fluid samples, and
Dⱼ is the standard deviation of the values of autoantibody abundance feature j in the training set of uterine lavage fluid samples.

43. The method of any one of claim 40-42, wherein the first set of autoantibody abundance features was identified from training data for a larger plurality of autoantibody abundance features using a feature extraction method.

44. The method of any one of claims 40-43, wherein each respective feature in the first set of autoantibody abundance features comprises a normalized abundance value for a respective autoantibody species in the first set of autoantibody species.

45. The method of any one of claims 40-43, wherein each respective feature in the first set of autoantibody abundance features comprises a comparison between an abundance value for a first respective autoantibody species in the first set of autoantibody species and an abundance value for a second respective autoantibody species in the first set of autoantibody species.

46. The method of any one of claim 40-45, wherein the first biological fluid sample comprises blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings.

47. The method of claim 46, wherein the first biological fluid sample is a uterine lavage fluid.

48. The method of any one of claims 40-47, wherein for each autoantibody species in the first set of autoantibody species, the corresponding abundance value for the respective autoantibody species comprises an abundance of IgG and IgA homologues of the first set of autoantibody species in the first biological fluid sample.

49. The method of any one of claims 40-48, wherein the first set of autoantibody species comprises at least 5 autoantibody species, wherein each respective autoantibody species of the at least 5 autoantibody species binds to a molecular target in a different pathway or cell type signature selected from those listed in Table 1.

50. The method of any one of claims 40-49, further comprising:
obtaining a second biological sample from the subject.

51. The method of claim 50, wherein the second biological sample is a fluid sample.

52. The method of claim 51, wherein the second biological sample comprises blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings.

53. The method of claim 52, wherein the fluid sample is a uterine lavage fluid or blood.

54. The method of any one of claims 50-53, wherein the autoantibody abundance dataset for the subject further comprises, for each autoantibody species in a second set of autoantibody species, a corresponding abundance value for the respective autoantibody species in the second biological sample.

55. The method of any one of claims 40-54, wherein the classifier was trained to distinguish between the at least two states of the disease condition based on at least abundance values for the first set of autoantibody species and one or more secondary features of the subject.

56. The method of claim 55, wherein:
the disease condition is an ovarian cancer or an endometrial cancer, and
the one or more secondary features of the subject comprise two or more of the features selected from the group consisting of an age of the subject, a pregnancy history of the subject, a breastfeeding history of the subject, a BRCA1 genotype of the subject, a BRCA2 genotype of the subject, a breast cancer history of the subject, and a familial history of endometrial cancer, ovarian cancer, or breast cancer.

57. The method of claim 55 or 56, further comprising:
obtaining nucleic acids from the first biological fluid sample or the second biological sample;
sequencing with a predetermined minimum coverage value the nucleic acid sequences targeted by a panel of genes, thereby obtaining a set of gene expression levels for the subject; and
inputting the set of gene expression levels into the classifier.

58. The method of claim 57, wherein the panel of genes comprises at least 2 genes, at least 5 genes, at least 10 genes, at least 15 genes, or at least 20 genes.

59. The method of any one of claims 40-58, wherein the disease condition is endometrial cancer.

60. The method of claim 59, wherein a stage of the disease is stage 0 endometrial cancer, stage IA endometrial cancer, stage IB endometrial cancer, stage II endometrial cancer, stage III endometrial cancer, or stage IV endometrial cancer.

61. The method of any one of claims 40-60, wherein the classifier comprises a molecular signature algorithm, a neural network algorithm, a support vector machine algorithm, a decision tree algorithm, an unsupervised clustering model algorithm, a supervised clustering model algorithm, or a regression model.

62. The method of any one of claims 40-61, wherein the determining b) comprises detectably binding each autoantibody to its cognate protein autoantigen.
